(19) United States
(12) Patent Application Publication
(10) Pub. No.: US 2024/0027334 A1
(43) Pub. Date: Jan. 25, 2024

(54) METHODS OF DETECTING SYNTHETIC URINE AND MATCHING A URINE SAMPLE TO A SUBJECT

(71) Applicant: Genotox Laboratories, Austin, TX (US)

(72) Inventors: Matt McCarty, Austin, TX (US); Keqin Gregg, Austin, TX (US)

(21) Appl. No.: 17/936,211

(22) Filed: Sep. 28, 2022

Related U.S. Application Data
(63) Continuation of application No. 14/752,511, filed on Jun. 26, 2015, now abandoned.
(60) Provisional application No. 62/018,330, filed on Jun. 27, 2014.

(51) Int. Cl. G01N 21/31; C12Q 1/6876

(52) U.S. Cl. CPC G01N 21/314; C12Q 1/6876; C12Q 2600/156

(57) ABSTRACT

Provided herein are methods for determining if a urine sample comprises synthetic urine, methods for matching a urine sample to a subject, and methods for amplifying DNA. Also provided are kits that include a set of at least 3 pairs of a pre-amplification forward and reverse primer, where each pair of pre-amplification forward and reverse primers is designed to amplify 250 to 300 nucleotides of genomic DNA that contains one of at least 3 SNPs, where the pre-amplification forward and reverse primers in each of the three or more pairs of pre-amplification primers contains (i) a sequence of about 17 to about 25 contiguous nucleotides that is complementary to a sequence in the genomic DNA and (i) a tag sequence of about 17 to about 25 contiguous nucleotides that is not complementary to a sequence in the genomic DNA.

### CROSS-REFERENCE TO RELATED APPLICATIONS

This application is a continuation of U.S. application Ser. No. 14/752,511, filed Jun. 26, 2015, which claims the benefit of U.S. Provisional Application 62/018,330, filed Jun. 27, 2014, which is considered part of (and is incorporated by reference in) the disclosure of this application.

### SEQUENCE LISTING

This application contains a Sequence Listing that has been submitted electronically as an XML file named “39930-0002002_SL_ST26.XML.” The XML file, created on Sep. 14, 2022, is 51,284 bytes in size. The material in the XML file is hereby incorporated by reference in its entirety.

### TECHNICAL FIELD

This invention relates to methods of molecular biology and urine testing.

### BACKGROUND

Urine drug testing is a commonly used tool to detect a subject's use of drugs, both legal (e.g., controlled substances) and illegal. During the last half century the use of urine drug testing has been used throughout the military, in the public and private workplace, in courts, and in medical clinics and care centers. The urine drug tests are used primarily to detect illegal or banned substances in a subject's system. In the clinical setting, physicians test their patients to determine if their patients are adhering to their prescriptions. Urine drug testing has become a routinely used effective tool in the assessment and ongoing management of patients who are treated with controlled substances for, e.g., chronic pain. The urine drug testing results provide confirmation of the agreed-upon treatment plan and diagnose relapse or drug abuse.

The results of a urine drug test can have serious consequences for a patient including termination of prescription. In fear of the possible consequences, patients have developed a variety of methods to cheat by substituting their own urine sample with that of others. Patients who “cheat” a urine drug test by using adulterated samples (e.g., another person's urine) or synthetic urine present a problem for the treating MD because the ongoing care plan will not be based on accurate information. Currently, the best method for validating that a patient's sample is in fact their own is by observation during sample collection—which is not always possible. Another complication of urine drug testing is that a clinical lab can mix-up urine samples, which also leads to inaccurate test results.

### SUMMARY

The present invention focuses on methods developed to determine the authenticity of a urine sample (e.g., used in association with drug testing or to achieve quality control). In view of this discovery, provided herein are methods of determining whether a urine sample comprises, consists essentially of, or consists of synthetic urine and methods of matching a urine sample to a subject. Also provided are methods of amplifying genomic DNA (e.g., genomic DNA isolated from cells enriched from a urine sample). Also provided are kits that include a set of at least two pairs (e.g., at least three pairs) of a pre-amplification forward and reverse primer, where each pair of pre-amplification forward and reverse primers is designed to amplify 100 to 500 nucleotides of genomic DNA (e.g., genomic DNA that contains at least one SNP or a site of a mutation), where the pre-amplification forward and reverse primers in each of the at least two pairs of pre-amplification primers contains (i) a sequence of about 10 to about 30 (e.g., about 17 to about 25) contiguous nucleotides that is complementary to a sequence in the genomic DNA and (i) a tag sequence of about 5 to about 25 (e.g., about 17 to about 25) contiguous nucleotides that is not complementary to a sequence in the genomic DNA.

Provided herein are methods of determining if a urine sample comprises, consists essentially of, or consists of synthetic urine that includes: (a) providing a urine sample from a subject; (b) enriching the urine sample for mammalian cells, if present; (c) isolating any genomic DNA from the enriched sample of step (b) to form an isolated genomic DNA test sample; (d) adding to the isolated genomic DNA test sample of step (c) a control DNA to form a control sample or adding the control DNA to the enriched sample of step (b) and then isolating DNA to form a control sample; (e) performing an assay to determine the presence of genomic DNA in the isolated genomic DNA sample of step (c) or the control sample of step (d); (f) performing an assay to determine the presence of the control DNA in the control sample of step (d); and (g) identifying a urine sample having no detectable level of genomic DNA and having detectable control DNA as comprising, consisting essentially or, or consisting of synthetic urine, or identifying a urine sample having a detectable level of genomic DNA and having detectable control DNA as not comprising a synthetic urine. In some embodiments of any of the methods described herein, the determination of the presence of genomic DNA includes performing an assay to determine the presence of at least three single nucleotide polymorphisms in the isolated genomic DNA sample of step (c) or the control sample of step (d), and a urine sample having no detectable level of the at least three SNPs and having detectable control DNA is identified in step (g) as containing synthetic urine, or a urine sample having a detectable level of the at least three SNPs and having detectable control DNA is identified in step (g) as not comprising synthetic urine. In some embodiments of any of the methods described herein, the urine sample is identified in step (g) as not comprising synthetic urine. Some embodiments of any of the methods described herein further include performing an assay to determine the level of one or more drugs and/or one or more drug metabolites in the urine sample identified in step (g) as not comprising synthetic urine. Some embodiments of any of the methods described herein further include: (h) performing an assay to determine the genotype of at least 6 single nucleotide polymorphisms (SNPs) in the isolated genomic DNA test sample of step (c) or the control sample of step (d); (i) comparing the genotype of the at least 6 SNPs in the isolated genomic DNA test sample of step (c) or the control sample of step (d) with the genotype of the at least 6 SNPs in a control cell sample from the subject; and (j) identifying a urine sample having a detectable level of the control DNA and having the same genotype of the at least 6 SNPs in the isolated genomic DNA test sample of step (c) or the control sample of step (d) as the genotype of the at least 6 SNPs in the control cell sample as originating from the subject; or identifying a urine sample having a detectable level of the control DNA and not having the same genotype of the at least 6 SNPs in the isolated genomic DNA test sample of (c) or the control sample of step (d) as the genotype of the at least 6 SNPs in the control cell sample as not originating from the subject. In some embodiments of any of the methods described herein, the control cell sample is a buccal cell sample. Some embodiments of any of the methods described herein, further include obtaining a control cell sample from the subject.

In some embodiments of any of the methods described herein, the at least 3 SNPs have a minor allele frequency of >0.4. In some embodiments of any of the methods described herein, the at least 3 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, the presence of at least six (e.g., at least ten or at least 14) SNPs, is determined. In some embodiments of any of the methods described herein, the at least three SNPs includes at least one SNP from at least two different chromosomes. In some embodiments of any of the methods described herein, the at least six SNPs includes at least one SNP from at least four different chromosomes. In some embodiments of any of the methods described herein, the at least fourteen SNPs includes at least one SNP from at least eight different chromosomes. In some embodiments of any of the methods described herein, the assay in step (e) includes a polymerase chain reaction (PCR) assay (e.g., real-time PCR assay). In some embodiments of any of the methods described herein, the assay in step (e) includes a pre-amplification step. In some embodiments of any of the methods described herein, the pre-amplification step includes: hybridization of three or more pairs of a pre-amplification forward and reverse primer, wherein each pair of pre-amplification forward and reverse primers is designed to amplify 250 to 300 nucleotides of genomic DNA that contains one of the at least 3 SNPs, wherein the pre-amplification forward and reverse primers in each of the three or more pairs of pre-amplification primers contain (i) a sequence of about 17 to about 25 contiguous nucleotides that is complementary to a sequence in the genomic DNA and (ii) a tag sequence of about 17 to about 25 contiguous nucleotides that is not complementary to a sequence in the genomic DNA; and amplification of the genomic DNA using the three or more pairs of pre-amplification forward and reverse primers to generate 250 to 300 nucleotide amplification product(s). In some embodiments of any of the methods described herein, the pre-amplification step further includes amplification of the 250 to 300 nucleotide amplification product(s) using a primer that includes a sequence of about 17 to about 25 contiguous nucleotides of the tag sequence. In some embodiments of any of the methods described herein, the tag sequence is CAAGATGCTACGCTTC AGTC (SEQ ID NO: 1). In some embodiments of any of the methods described herein, the three or more pairs of pre-amplification reverse and forward primers are selected from the group of: (i) SEQ ID NO: 2 and SEQ ID NO: 3, respectively; (ii) SEQ ID NO: 4 and SEQ ID NO: 5, respectively; (iii) SEQ ID NO: 6 and SEQ ID NO: 7, respectively; (iv) SEQ ID NO: 8 and SEQ ID NO: 9, respectively; (v) SEQ ID NO: 10 and SEQ ID NO: 11, respectively; (vi) SEQ ID NO: 12 and SEQ ID NO: 13, respectively; (vii) SEQ ID NO: 14 and SEQ ID NO: 15, respectively; (viii) SEQ ID NO: 16 and SEQ ID NO: 17, respectively; (ix) SEQ ID NO: 18 and SEQ ID NO: 19, respectively; (xii) SEQ ID NO: 20 and SEQ ID NO: 21, respectively; (xiii) SEQ ID NO: 22 and SEQ ID NO: 23, respectively; (xiv) SEQ ID NO: 24 and SEQ ID NO: 25, respectively; (xv) SEQ ID NO: 26 and SEQ ID NO: 27, respectively; and (xvi) SEQ ID NO: 28 and SEQ ID NO: 29, respectively.

In some embodiments of any of the methods described herein, the control DNA is a plant DNA (e.g., a cDNA encoding spinach chloroplast ATP synthase gamma-subunit (AtpC)). In some embodiments of any of the methods described herein, the assay in step (f) includes a polymerase chain reaction (PCR) assay (e.g., a real-time PCR assay). In some embodiments of any of the methods described herein, the control DNA is a cDNA encoding spinach chloroplast ATP synthase gamma-subunit (AtpC) and the PCR assay utilizes forward and reverse primers having the sequence of SEQ ID NO: 36 and SEQ ID NO: 37, respectively. In some embodiments of any of the methods described herein, the subject is a human.

Some embodiments of any of the methods described herein further include (h) performing an assay to identify the presence of one or more of statherin, alpha-amylase, and lysozyme in the urine sample; and (i) identifying a urine sample having a detectable level of genomic DNA, a detectable control DNA, and a detectable level of one or more of statherin, alpha-amylase, and lysozyme as being adulterated. In some embodiments of any of the methods described herein, the assay in step (h) is an enzyme activity assay. In some embodiments of any of the methods described herein, the assay in step (h) is an enzyme-linked immunosorbent assay (ELISA). Some embodiments of any of the methods described herein, further include recording the identification in step (g) in the subject's medical record. In some embodiments of any of the methods described herein, the subject's medical record is a computer readable medium. Some embodiments of any of the methods described herein further include notifying the subject's insurance provider, employer, or potential future employer of the identification in step (g). Some embodiments of any of the methods described herein further include notifying a pharmacist or a medical professional of the identification in step (g). Some embodiments of any of the methods described herein further include: (h) selecting a subject having a urine sample identified in step (g) as containing synthetic urine; and (i) obtaining an additional urine sample from the selected subject. In some embodiments of any of the methods described herein, the additional urine sample is obtained through a witnessed urine test. Some embodiments of any of the methods described herein further include (j) performing an assay to determine the level of one or more drugs and/or the level of one or more drug metabolites in the additional urine sample. Some embodiments of any of these methods further include: (k) identifying a subject having an elevated level of one or more drugs and/or an elevated level of one or more drug metabolites in the additional urine sample as compared to a reference level of the one or more drugs and/or a reference level of the one or more drug metabolites, wherein the drugs are an illegal or controlled substance and/or the drug metabolites are metabolites of an illegal or controlled substance; and (l) admitting the subject into a drug dependency program, ceasing administration of the controlled substance to the subject, or reducing the dose and/or frequency of administration of the controlled substance to the subject. In some embodiments of any of the methods described herein, the drug dependency program includes administering to the subject in step (l) a drug replacement therapy.

Some embodiments of any of the methods described herein further include: (h) selecting a subject having a urine sample identified in step (g) as containing synthetic urine; (i) obtaining a sample comprising blood, serum, hair, or plasma from the subject; and (j) performing an assay to determine the level of one or more drugs and/or one or more drug metabolites in the sample from step (i). Some embodiments of any of the methods described herein further include: (k) identifying a subject having an elevated level of one or more drugs and/or an elevated level of one or more drug metabolites in the sample from step (i) as compared to a reference level of the one or more drugs and/or a reference level of the one or more drug metabolites, wherein the drugs are an illegal or controlled substance and/or the drug metabolites are metabolites of an illegal or controlled substance; and (l) admitting the subject into a drug dependency program, ceasing administration of the controlled substance to the subject, or reducing the dose or frequency of administration of the controlled substance to the subject. In some embodiments of any of the methods described herein, the drug dependency program includes administering to the subject in step (l) a drug replacement therapy.

In some embodiments of any of the methods described herein, the subject has not been diagnosed as having an illegal or controlled substance addiction. In some embodiments of any of the methods described herein, the subject has been identified as having an illegal or controlled substance addiction. In some embodiments of any of the methods described herein, the subject is being treated on an outpatient basis for an illegal or controlled substance addiction.

Also provided herein are methods of determining if a urine sample comprises, consists essentially of, or consists of synthetic urine and/or is diluted that include: (a) providing a urine sample from a subject; (b) detecting the absorbance at 280 nm of the urine sample; and (c) identifying a urine sample having an absorbance at 280 nm that is less than a reference 280 nm absorbance value as comprising, consisting essentially of, or consisting of synthetic urine and/or being diluted, or identifying a urine sample having an absorbance at 280 nm that is equal to or greater than the reference 280 nm absorbance value as not comprising synthetic urine and not being diluted. Some embodiments of any of the methods described herein further include, after step (a) and before step (b), centrifuging the urine sample to remove particulate matter. Some embodiments of any of the methods described herein further include: (d) determining the absorbance at 240 nm of the urine sample; and (e) further identifying a urine sample having an absorbance at 280 nm that is less than a reference 280 nm absorbance value and an absorbance at 240 nm that is less than a reference 240 nm absorbance value as being diluted.

In some embodiments of any of the methods described herein, the urine sample is identified in step (c) as not comprising synthetic urine and not being diluted. Some embodiments of any of the methods described herein further include performing an assay to determine the level of one or more drugs and/or one or more drug metabolites in the urine sample identified in step (c) as not comprising synthetic urine and not being diluted. Some embodiments of any of the methods described herein further include: (d) enriching the urine sample for mammalian cells, if present; (e) isolating any genomic DNA from the enriched sample of step (d) to form an isolated genomic DNA test sample; (f) adding to the isolated genomic DNA test sample of step (e) a control DNA to form a control sample or adding the control DNA to the enriched sample of step (d) and then isolating the DNA to form a control sample; (g) performing an assay to determine the genotype of at least 6 single nucleotide polymorphisms (SNPs) in the isolated genomic DNA test sample of step (e) or the control sample of step (f); (h) comparing the genotype of the at least 6 SNPs in the isolated genomic DNA test sample of step (e) or the control sample of step (f) with the genotype of the at least 6 SNPs in a control cell sample from the subject; (i) performing an assay to determine the presence of the control DNA in the control sample of step (f); and (j) identifying a urine sample having a detectable level of the control DNA and having the same genotype of the at least 6 SNPs in the isolated genomic DNA test sample of step (e) or the control sample of step (f) as the genotype of the at least 6 SNPs in the control cell sample as originating from the subject; or identifying a urine sample having a detectable level of the control DNA and not having the same genotype of the at least 6 SNPs in the isolated genomic DNA test sample of step (e) or the control sample of step (f) as the genotype of the at least 6 SNPs in the control cell sample as not originating from the subject.

In some embodiments of any of the methods described herein, the at least 6 (e.g., at least 8, at least 10, or at least 14) SNPs in step (g) have a minor allele frequency of >0.4. In some embodiments of any of the methods described herein, the at least 6 SNPs are selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments of any of the methods described herein, the at least 6 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, the at least 8 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, the at least 10 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, in step (e) the genotype of rs7520386, rs560681, rs9951171, rs1058083, rs1358856, rs214955, rs740598, rs279844, rs13218440, rs2272998, rs12997453, rs13134862, rs13182883, and rs1410059 are determined. In some embodiments of any of the methods described herein, the subject is a genetic male, at least one of the SNPs in step (g) is located on a Y chromosome, and no detectable level of the at least one of the SNPs located on the Y chromosome further identifies the urine sample as not originating from the subject.

In some embodiments of any of the methods described herein, the genotype of at least eight (e.g., at least ten or at least fourteen) SNPs are determined in step (g). In some embodiments of any of the methods described herein, the at least six SNPs in step (g) includes at least one SNP from at least three different chromosomes. In some embodiments of any of the methods described herein, the at least ten SNPs includes at least one SNP from at least six different chromosomes. In some embodiments of any of the methods described herein, the at least fourteen SNPs includes at least one SNP from at least eight different chromosomes.

In some embodiments of any of the methods described herein, the assay in step (g) includes a polymerase chain reaction (PCR) assay (e.g., a real-time PCR assay). In some embodiments of any of the methods described herein, the assay in step (g) includes a pre-amplification step. In some embodiments of any of the methods described herein, the pre-amplification step includes: hybridization of six or more pairs of a pre-amplification forward and reverse primer, wherein each pair of pre-amplification forward and reverse primers is designed to amplify 250 to 300 nucleotides of genomic DNA that contains one of the at least 6 SNPs, wherein the pre-amplification forward and reverse primers in each of the six or more pairs of pre-amplification primers contains (i) a sequence of about 17 to about 25 contiguous nucleotides that is complementary to a sequence in the genomic DNA and (i) a tag sequence of about 17 to about 25 contiguous nucleotides that is not complementary to a sequence in the genomic DNA; and amplification of the genomic DNA using the six or more pairs of pre-amplification forward and reverse primers to generate 250 to 300 nucleotide amplification product(s). In some embodiments of any of the methods described herein, the pre-amplification step further includes amplification of the 250 to 300 nucleotide amplification product(s) using a primer that includes a sequence of about 17 to about 25 contiguous nucleotides of the tag sequence. In some embodiments of any of the methods described herein, the tag sequence is CAAGATGCTACGCTTC AGTC (SEQ ID NO: 1). In some embodiments of any of the methods described herein, the six or more pairs of pre-amplification reverse and forward primers are selected from the group of: (i) SEQ ID NO: 2 and SEQ ID NO: 3, respectively; (ii) SEQ ID NO: 4 and SEQ ID NO: 5, respectively; (iii) SEQ ID NO: 6 and SEQ ID NO: 7, respectively; (iv) SEQ ID NO: 8 and SEQ ID NO: 9, respectively; (v) SEQ ID NO: 10 and SEQ ID NO: 11, respectively; (vi) SEQ ID NO: 12 and SEQ ID NO: 13, respectively; (vii) SEQ ID NO: 14 and SEQ ID NO: 15, respectively; (viii) SEQ ID NO: 16 and SEQ ID NO: 17, respectively; (ix) SEQ ID NO: 18 and SEQ ID NO: 19, respectively; (xii) SEQ ID NO: 20 and SEQ ID NO: 21, respectively; (xiii) SEQ ID NO: 22 and SEQ ID NO: 23, respectively; (xiv) SEQ ID NO: 24 and SEQ ID NO: 25, respectively; (xv) SEQ ID NO: 26 and SEQ ID NO: 27, respectively; (xvi) SEQ ID NO: 28 and SEQ ID NO: 29, respectively; (xvii) SEQ ID NO: 32 and SEQ ID NO: 33, respectively; and (xviii) SEQ ID NO: 34 and SEQ ID NO: 35, respectively.

In some embodiments of any of the methods described herein, the control DNA is plant DNA (e.g., a cDNA encoding spinach chloroplast ATP synthase gamma-subunit (AtpC)). In some embodiments of any of the methods described herein, the assay in step (i) includes a polymerase chain reaction (PCR) assay (e.g., a real-time PCR assay). In some embodiments of any of the methods described herein, the control DNA is a cDNA encoding spinach chloroplast ATP synthase gamma-subunit (AtpC) and the PCR assay utilizes forward and reverse primers having the sequence of SEQ ID NO: 36 and SEQ ID NO: 37, respectively. In some embodiments of any of the methods described herein, the control cell sample is a buccal cell sample. Some embodiments of any of the methods described herein further include a step of obtaining a control cell sample from a subject. Some embodiments of any of the methods described herein further include determining the genotype of the at least 6 SNPs in the control cell sample. In some embodiments of any of the methods described herein, the subject is a human.

Some embodiments of any of the methods described herein, further include: (k) performing an assay to identify the presence of one or more of statherin, alpha-amylase, and lysozyme in the urine sample; and (l) identifying a urine sample having a genotype of the at least 6 SNPs in the isolated genomic DNA test sample of step (c) or the control sample of step (d) that is the same as the genotype of the 6 SNPs in the control cell sample, a detectable level of control DNA, and a detectable level of one or more of statherin, alpha-amylase, and lysozyme as being adulterated. In some embodiments of any of the methods described herein, the assay in step (k) is an enzyme activity assay. In some embodiments of any of the methods described herein, the assay in step (k) is an enzyme-linked immunosorbent assay (ELISA).

Some embodiments of any of the methods described herein further include recording the identification in step (c) in the subject's medical record. Some embodiments of any of the methods described herein further include recording the identification in step (e) in the subject's medical record. Some embodiments of any of the methods described herein further include recoding the identification in step (j) in the subject's medical record. In some embodiments of any of the methods described herein, the subject's medical record is a computer readable medium. Some embodiments of any of the methods described herein further include notifying the subject's insurance provider, employer, or potential future employer of the identification in step (c). Some embodiments of any of the methods described herein further include notifying the subject's insurance provider, employer, or potential future employer of the identification in step (e). Some embodiments of any of the methods described herein further include notifying the subject's insurance provider, employer, or potential future employer of the identification in step (j). Some embodiments of any of the methods described herein further include notifying a pharmacist or a medical professional of the identification in step (c). Some embodiments of any of the methods described herein further include notifying a pharmacist or a medical professional of the identification in step (e). Some embodiments of any of the methods described herein further include notifying a pharmacist or a medical professional of the identification in step (j). Some embodiments of any of the methods described herein further include: (d) selecting a subject having a urine sample identified in step (c) as comprising synthetic urine and/or being diluted; and (e) obtaining an additional urine sample from the subject. Some embodiments of any of the methods described herein further include: (f) selecting a subject having a urine sample identified in step (e) as being diluted; and (g) obtaining an additional urine sample from the subject. Some embodiments of any of the methods described herein further include: (k) selecting a subject having a urine sample identified in step j) as not originating from the subject; and (l) obtaining an additional urine sample from the selected subject. In some embodiments of any of the methods described herein, the additional urine sample is obtained through a witnessed urine test. Some embodiments of any of the methods described herein further include performing an assay to determine the level of one or more drugs and/or one or more drug metabolites in the additional urine sample. Some embodiments of any of the methods described herein further include: identifying a subject having an elevated level of one or more drugs and/or an elevated level of one or more drug metabolites in the additional urine sample as compared to a reference level of the one or more drugs and/or a reference level of one or more drug metabolites, wherein the drugs are an illegal or controlled substance and/or the drug metabolites are metabolites of an illegal or controlled substance; and admitting the identified subject into a drug dependency program, ceasing administration of the controlled substance to the identified subject, or reducing the dose and/or frequency of administration of the controlled substance to the identified subject. In some embodiments of any of the methods described herein, the drug dependency program includes administering to the admitted subject a drug replacement therapy.

Some embodiments of any of the methods described herein further include: (d) selecting a subject having a urine sample identified in step (c) as comprising synthetic urine and/or being diluted; (e) obtaining an additional sample comprising blood, serum, hair, or plasma from the subject; and (f) performing an assay to determine the level of one or more drugs and/or the level of one or more drug metabolites in the additional sample from step (e). Some embodiments of any of the methods described herein include: (f) selecting a subject having a urine sample identified in step (e) as being diluted; (g) obtaining an additional sample comprising blood, serum, hair, or plasma from the subject; and (h) performing an assay to determine the level of one or more drugs and/or the level of one or more drug metabolites in the additional sample from step (g). Some embodiments of any of the methods described herein further include: (k) selecting a subject having a urine sample identified in step (j) as not originating from the subject; (l) obtaining an additional sample comprising blood, serum, hair, or plasma from the subject; and (m) performing an assay to determine the level of one or more drugs and/or the level of one or more drug metabolites in the additional sample from step (l). Some embodiments of any of the methods described herein further include: identifying a subject having an elevated level of one or more drugs and/or an elevated level of one or more drug metabolites in the additional sample as compared to a reference level of the one or more drugs and/or a reference level of the one or more drug metabolites, wherein the drugs are an illegal or controlled substance and/or the drug metabolites are metabolites of an illegal or controlled substance; and admitting the identified subject into a drug dependency program, ceasing administration of the controlled substance to the identified subject, or reducing the dose or frequency of administration of the controlled substance to the identified subject. In some embodiments of any of the methods described herein, the drug dependency program includes administering to the admitted subject a drug replacement therapy.

In some embodiments of any of the methods described herein, the subject has not been diagnosed as having an illegal or controlled substance addiction. In some embodiments of any of the methods described herein, the subject has been identified as having an illegal or controlled substance addiction. In some embodiments of any of the methods described herein, the subject is being treated on an outpatient basis for an illegal or controlled substance addiction.

Also provided herein are methods of matching a urine sample to a subject that include: (a) providing a urine sample from a subject; (b) enriching the urine sample for mammalian cells, if present; (c) isolating any genomic DNA from the enriched sample of step (b) to form an isolated genomic DNA test sample; (d) adding to the isolated genomic DNA test sample of step (c) a control DNA to form a control sample or adding the control DNA to the enriched sample of step (b) and then isolating the DNA to form a control sample; (e) performing an assay to determine the genotype of at least 6 single nucleotide polymorphisms (SNPs) in the isolated genomic DNA test sample of step (c) or the control sample of step (d); (f) comparing the genotype of the at least 6 SNPs in the isolated genomic DNA test sample of step (c) or the control sample of step (d) with the genotype of the at least 6 SNPs in a control cell sample from the subject; (g) performing an assay to determine the presence of the control DNA in the control sample of step (d); and (h) identifying a urine sample having a detectable level of the control DNA and having the same genotype of the at least 6 SNPs in the isolated genomic DNA test sample of step (c) or the control sample of step (d) as the genotype of the at least 6 SNPs in the control cell sample as originating from the subject; or identifying a urine sample having a detectable level of the control DNA and not having the same genotype of the at least 6 SNPs in the isolated genomic DNA test sample of step (c) or the control sample of step (d) as the genotype of the at least 6 SNPs in the control cell sample as not originating from the subject.

In some embodiments of any of the methods described herein, the urine sample is identified in step (h) as originating from the subject. Some embodiments of any of the methods described herein further include performing an assay to determine the level of one or more drugs and/or one or more drug metabolites in the urine sample identified in step (h) as originating from the subject. In some embodiments of any of the methods described herein, the at least 6 (e.g., at least 8, at least 10, or at least 14) SNPs in step (e) have a minor allele frequency of >0.4. In some embodiments of any of the methods described herein, the at least 6 SNPs are selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments of any of the methods described herein, the at least 6 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, the at least 8 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, the at least 10 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the methods described herein, in step (e) the genotype of rs7520386, rs560681, rs9951171, rs1058083, rs1358856, rs214955, rs740598, rs279844, rs13218440, rs2272998, rs12997453, rs13134862, rs13182883, and rs1410059 are determined. In some embodiments of any of the methods described herein, the subject is a genetic male, at least one of the SNPs in step (e) is located on a Y chromosome, and no detectable level of the at least one of the SNPs located on the Y chromosome further identifies the urine sample as not originating from the subject.

In some embodiments of any of the methods described herein, the genotype of at least eight (e.g., at least 10 or at least 14) SNPs are determined in step (e). In some embodiments of any of the methods described herein, the at least six SNPs in step (e) includes at least one SNP from at least three different chromosomes. In some embodiments of any of the methods described herein, the at least ten SNPs includes at least one SNP from at least six different chromosomes. In some embodiments of any of the methods described herein, the at least fourteen SNPs includes at least one SNP from at least eight different chromosomes.

In some embodiments of any of the methods described herein, the assay in step (e) includes a polymerase chain reaction (PCR) assay (e.g., real-time PCR assay). In some embodiments of any of the methods described herein, the assay in step (e) includes a pre-amplification step. In some embodiments of any of the methods described herein, the pre-amplification step includes: hybridization of six or more pairs of a pre-amplification forward and reverse primer, wherein each pair of pre-amplification forward and reverse primers is designed to amplify 250 to 300 nucleotides of genomic DNA that contains one of the at least 6 SNPs, wherein the pre-amplification forward and reverse primers in each of the six or more pairs of pre-amplification primers contains (i) a sequence of about 17 to about 25 contiguous nucleotides that is complementary to a sequence in the genomic DNA and (i) a tag sequence of about 17 to about 25 contiguous nucleotides that is not complementary to a sequence in the genomic DNA; and amplification of the genomic DNA using the six or more pairs of pre-amplification forward and reverse primers to generate 250 to 300 nucleotide amplification product(s). In some embodiments of any of the methods described herein, the pre-amplification step further includes amplification of the 250 to 300 nucleotide amplification product(s) using a primer that includes a sequence of about 17 to about 25 contiguous nucleotides of the tag sequence. In some embodiments of any of the methods described herein, the tag sequence is CAAGATGCTACGCTTCAGTC (SEQ ID NO: 1). In some embodiments of any of the methods described herein, the six or more pairs of pre-amplification reverse and forward primers are selected from the group of: (i) SEQ ID NO: 2 and SEQ ID NO: 3, respectively; (ii) SEQ ID NO: 4 and SEQ ID NO: 5, respectively; (iii) SEQ ID NO: 6 and SEQ ID NO: 7, respectively; (iv) SEQ ID NO: 8 and SEQ ID NO: 9, respectively; (v) SEQ ID NO: 10 and SEQ ID NO: 11, respectively; (vi) SEQ ID NO: 12 and SEQ ID NO: 13, respectively; (vii) SEQ ID NO: 14 and SEQ ID NO: 15, respectively; (viii) SEQ ID NO: 16 and SEQ ID NO: 17, respectively; (ix) SEQ ID NO: 18 and SEQ ID NO: 19, respectively; (xii) SEQ ID NO: 20 and SEQ ID NO: 21, respectively; (xiii) SEQ ID NO: 22 and SEQ ID NO: 23, respectively; (xiv) SEQ ID NO: 24 and SEQ ID NO: 25, respectively; (xv) SEQ ID NO: 26 and SEQ ID NO: 27, respectively; (xvi) SEQ ID NO: 28 and SEQ ID NO: 29, respectively; (xvii) SEQ ID NO: 32 and SEQ ID NO: 33, respectively; and (xviii) SEQ ID NO: 34 and SEQ ID NO: 35, respectively.

In some embodiments of any of the methods described herein, the control DNA is plant DNA (e.g., a cDNA encoding spinach chloroplast ATP synthase gamma-subunit (AtpC)). In some embodiments of any of the methods described herein, the assay in step (g) includes a polymerase chain reaction (PCR) assay (e.g., a real-time PCR assay). In some embodiments of any of the methods described herein, the control DNA is a cDNA encoding spinach chloroplast ATP synthase gamma-subunit (AtpC) and the PCR assay utilizes forward and reverse primers having the sequence of SEQ ID NO: 36 and SEQ ID NO: 37, respectively.

In some embodiments of any of the methods described herein, the control cell sample is a buccal cell sample. Some embodiments of any of the methods described herein further include a step of obtaining a control cell sample from a subject. Some embodiments of any of the methods described herein further include determining the genotype of the at least 6 SNPs in the control cell sample. In some embodiments of any of the methods described herein, the subject is a human.

Some embodiments of any of the methods described herein further include: (i) performing an assay to identify the presence of one or more of statherin, alpha-amylase, and lysozyme in the urine sample; and (j) identifying a urine sample having a genotype of the at least 6 SNPs in the isolated genomic DNA test sample of step (c) or the control sample of step (d) that is the same as the genotype of the 6 SNPs in the control cell sample, a detectable level of control DNA, and a detectable level of one or more of statherin, alpha-amylase, and lysozyme as being adulterated. In some embodiments of any of the methods described herein, the assay in step (i) is an enzyme activity assay. In some embodiments of any of the methods described herein, the assay in step (i) is an enzyme-linked immunosorbent assay (ELISA).

Some embodiments of any of the methods described herein further include recording the identification in step (h) in the subject's medical record. In some embodiments of any of the methods described herein, the subject's medical record is a computer readable medium. Some embodiments of any of the methods described herein further include notifying the subject's insurance provider, employer, or potential future employer of the identification in step (h). Some embodiments of any of the methods described herein further include notifying a pharmacist or a medical professional of the identification in step (h). Some embodiments of any of the methods described herein further include (i) selecting a subject having a urine sample identified in step (h) as not originating from the subject; and (j) obtaining an additional urine sample from the selected subject. In some embodiments of any of the methods described herein, the additional urine sample is obtained through a witnessed urine test. Some embodiments of any of the methods described herein further include (k) performing an assay to determine the level of one or more drugs and/or one or more drug metabolites in the additional urine sample. Some embodiments of any of the methods described herein further include: (l) identifying a subject having an elevated level of one or more drugs and/or an elevated level of one or more drug metabolites in the additional urine sample as compared to a reference level of the one or more drugs and/or a reference level of one or more drug metabolites, wherein the drugs are an illegal or controlled substance and/or the drug metabolites are metabolites of an illegal or controlled substance; and (m) admitting the subject into a drug dependency program, ceasing administration of the controlled substance to the subject, or reducing the dose and/or frequency of administration of the controlled substance to the subject. In some embodiments of any of the methods described herein, the drug dependency program includes administering to the subject in step (m) a drug replacement therapy.

Some embodiments of any of the methods described herein further include: (i) selecting a subject having a urine sample identified in step (h) as not originating from the subject; (j) obtaining a sample comprising blood, serum, hair, or plasma from the subject; and (k) performing an assay to determine the level of one or more drugs and/or the level of one or more drug metabolites in the sample from step (i). Some embodiments of any of the methods described herein further include: (l) identifying a subject having an elevated level of one or more drugs and/or an elevated level of one or more drug metabolites in the sample from step (j) as compared to a reference level of the one or more drugs and/or a reference level of the one or more drug metabolites, wherein the drugs are an illegal or controlled substance and/or the drug metabolites are metabolites of an illegal or controlled substance; and (m) admitting the subject into a drug dependency program, ceasing administration of the controlled substance to the subject, or reducing the dose or frequency of administration of the controlled substance to the subject. In some embodiments of any of the methods described herein, the drug dependency program includes administering to the subject in step (m) a drug replacement therapy.

In some embodiments of any of the methods described herein, the subject has not been diagnosed as having an illegal or controlled substance addiction. In some embodiments of any of the methods described herein, the subject has been identified as having an illegal or controlled substance addiction. In some embodiments of any of the methods described herein, the subject is being treated on an outpatient basis for an illegal or controlled substance addiction.

Also provided herein are kits comprising, consisting essentially of, or consisting of: (i) a set of at least 3 pairs of a pre-amplification forward and reverse primer, wherein each pair of pre-amplification forward and reverse primers is designed to amplify 250 to 300 nucleotides of genomic DNA that contains one of at least 3 SNPs, wherein the pre-amplification forward and reverse primers in each of the three or more pairs of pre-amplification primers contains (i) a sequence of about 17 to about 25 contiguous nucleotides that is complementary to a sequence in the genomic DNA and (i) a tag sequence of about 17 to about 25 contiguous nucleotides that is not complementary to a sequence in the genomic DNA; and (ii) a primer that includes a sequence of about 17 to about 25 contiguous nucleotides of the tag sequence. Some embodiments of any of the kits described herein further include: an enzyme-linked immunosorbent assay for detection of one or more of statherin, amylase, and lysozyme, and/or a labeled substrate for detection of the activity of one or more of statherin, amylase, and lysozyme. In some embodiments of any of the kits described herein, the tag sequence is CAAGATGCTACGCTTC AGTC (SEQ ID NO: 1). In some embodiments of any of the kits described herein, the at least 3 (e.g., at least 6, at least 8, at least 10, or at least 14) SNPs in (i) have a minor allele frequency of >0.4.

In some embodiments of any of the kits described herein, the at least 6 SNPs are selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some embodiments of any of the kits described herein, the at least 6 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the kits described herein, the at least 8 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the kits described herein, at least 10 SNPs are selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some embodiments of any of the kits described herein, the SNPs in (i) include rs7520386, rs560681, rs9951171, rs1058083, rs1358856, rs214955, rs740598, rs279844, rs13218440, rs2272998, rs12997453, rs13134862, rs13182883, and rs1410059.

In some embodiments of any of the kits described herein, the SNPs in (i) include at least one (e.g., at least two) SNP(s) located on the Y chromosome. In some embodiments of any of the kits described herein, (i) includes at least 8 pairs of pre-amplification forward and reverse primers that amplify at least 8 SNPs. In some embodiments of any of the kits described herein, (i) includes at least 10 pairs of pre-amplification forward and reverse primers that amplify at least 10 SNPs. In some embodiments of any of the kits described herein, (i) includes at least 14 pairs of pre-amplification forward and reverse primers that amplify at least 14 SNPs.

In some embodiments of any of the kits described herein, the at least 8 SNPs includes at least one SNP from at least three different chromosomes. In some embodiments of any of the kits described herein, the at least ten SNPs includes at least one SNP from at least six different chromosomes. In some embodiments of any of the kits described herein, the at least fourteen SNPs includes at least one SNP from at least eight different chromosomes. In some embodiments of any of the kits described herein, the at least three pairs of pre-amplification reverse and forward primers are selected from the group of: (i) SEQ ID NO: 2 and SEQ ID NO: 3, respectively; (ii) SEQ ID NO: 4 and SEQ ID NO: 5, respectively; (iii) SEQ ID NO: 6 and SEQ ID NO: 7, respectively; (iv) SEQ ID NO: 8 and SEQ ID NO: 9, respectively; (v) SEQ ID NO: 10 and SEQ ID NO: 11, respectively; (vi) SEQ ID NO: 12 and SEQ ID NO: 13, respectively; (vii) SEQ ID NO: 14 and SEQ ID NO: 15, respectively; (viii) SEQ ID NO: 16 and SEQ ID NO: 17, respectively; (ix) SEQ ID NO: 18 and SEQ ID NO: 19, respectively; (xii) SEQ ID NO: 20 and SEQ ID NO: 21, respectively; (xiii) SEQ ID NO: 22 and SEQ ID NO: 23, respectively; (xiv) SEQ ID NO: 24 and SEQ ID NO: 25, respectively; (xv) SEQ ID NO: 26 and SEQ ID NO: 27, respectively; (xvi) SEQ ID NO: 28 and SEQ ID NO: 29, respectively; (xvii) SEQ ID NO: 32 and SEQ ID NO: 33, respectively; and (xviii) SEQ ID NO: 34 and SEQ ID NO: 35, respectively.

Some embodiments of any of the kits described herein, further include a control DNA. In some embodiments of any of the kits described herein, the control DNA is a plant DNA (e.g., a cDNA encoding spinach chloroplast ATP synthase gamma-subunit (AtpC)). Some embodiments of any of the kits described herein further include forward and reverse primers for amplifying the control DNA. In some embodiments of any of the kits described herein, the forward and reverse primers have the sequence of SEQ ID NO: 36 and SEQ ID NO: 37, respectively.

Also provided herein are methods for amplifying DNA that include: hybridizing six or more pairs of a pre-amplification forward and reverse primer, wherein each pair of pre-amplification forward and reverse primers is designed to amplify 250 to 300 nucleotides of genomic DNA that contains one of the at least 6 SNPs, wherein the pre-amplification forward and reverse primers in each of the six or more pairs of pre-amplification primers contains (i) a sequence of about 17 to about 25 contiguous nucleotides that is complementary to a sequence in the genomic DNA and (i) a tag sequence of about 17 to about 25 contiguous nucleotides that is not complementary to a sequence in the genomic DNA; amplifying the genomic DNA using the six or more pairs of pre-amplification forward and reverse primers to generate 250 to 300 nucleotide amplification product(s); and amplifying the 250 to 300 nucleotide amplification product(s) using a single generic primer that includes a sequence of about 17 to about 25 contiguous nucleotides of the tag sequence.

As used herein, the word “a” before a noun represents one or more of the particular noun. For example, the phrase “a SNP” represents “one or more SNPs.”

The term “subject” means a vertebrate, including any member of the class mammalia, including humans, sports or pet animals, such as horse (e.g., race horse) or dog (e.g., race dogs), and higher primates. In preferred embodiments, the subject is a human.

The term “control DNA” means an isolated contiguous DNA sequence that is not found in the genome of the subject (e.g., a human). For example, a control DNA can be an isolated contiguous DNA sequence not found in the genome of a mammalian cell (e.g., a contiguous DNA sequence that is not found in a human cell). For example, a control DNA can also be a DNA sequence that is not found in the genome of a bacterium (e.g., a Gram positive bacterium, a Gram negative bacterium, and a mycobacterium).

The term “synthetic urine” is art known and means a synthetic liquid that is not produced by the body of a mammal (e.g., human) that is meant to substitute for urine produced by the body of a mammal (e.g., a human). As is known in the art, synthetic urine is commercially available from a number of vendors.

The phrase “enriching a urine sample for mammalian cells, if present” means handling or processing a sample of urine in order to concentrate any mammalian cells, if present, in the sample. Non-limiting methods for enriching a urine sample for mammalian cells, if present, can include one or more steps of centrifugation (e.g., high speed centrifugation), beads coated with an antibody that specifically binds to an antigen present on the surface of mammalian cells, filtration, gravitational settling of the sample, and aspiration or removal of a supernatant substantially free of mammalian cells.

The term “drug metabolite” is art known and means a break-down product of a controlled or illegal substance produced by a mammal's body following administration of the controlled or illegal substance to the mammal (e.g., human). A wide variety of drugs, drug metabolites, and assays for detecting the levels of drugs and drug metabolites are known in the art. Non-limiting examples of drugs, drug metabolites, and vendors that sell kits for determining the level of one or more drugs and drug metabolites are described herein.

The term “control cell sample” means a biological sample obtained from the body of a subject, other than a urine sample, that includes a plurality of mammalian cells. Non-limiting examples of control cell samples include a hair sample, a blood sample, a buccal cell sample, mucus, phlegm, skin cells, tears, and saliva. Additional control cell samples are known in the art.

The phrase “originating from the subject” means a material or sample produced by the subject's body and not produced by another subject's body.

The phrase “not originating from the subject” means a material or sample produced by different subject's body.

The term “adulterated sample” means a urine sample (e.g., synthetic urine sample) from a subject that has been manipulated to add genomic DNA from the subject, where the added genomic DNA is from a source other than mammalian cells present in urine.

The term “potential future employer” means a person or business entity that is considering a subject for employment and that requires or asks employment candidates to provide a urine sample for testing as part of the job application process. For example, a potential future employer can be a state or federal government, a medical care facility (e.g., a clinic or a hospital), a transportation company, or an airline company.

The term “controlled substance” means an agent or material that is regulated by a government (e.g., state, federal government, or a governmental drug regulatory agency, such as the U.S. Food and Drug Administration), but its administration to at least some persons is not illegal. For example, the dosage and frequency of administration of a controlled substance can be regulated by a government. In some examples, certain persons in a population are warned not to consume a controlled substance. Non-limiting examples of controlled substances are prescription drugs and marijuana.

The term “drug replacement therapy” means administration of an agent that mimics the pharmacological effect of a controlled or illegal substance but is longer acting, less potent, less toxic, and/or has an improved safety profile than the controlled or illegal substance.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a flow chart of the non-limiting exemplary method performed in Example 1.

FIG. 2 is a graph showing the amplification of the spinach AtpC gene (fluorescence signal) over time in a real-time PCR assay. The blue data are generated from samples containing specific forward and reverse AtpC primers and the spinach genomic DNA. The amplification of the spinach AtpC gene is a 108 base pair fragment, which is detected using a TaqMan probe (TCCACAATTCCAACACCCTCCTCC; SEQ ID NO: 41) labeled with FAM. The TaqMan probe is designed to hybridize with the center of the amplified product. The red data is the base line absorbance generated by the fluorescence dye Rox in the reaction mixture. The green data represents a second probe for a genotyping assay which is not used in the assay and is used to measure the background absorbance.

FIG. 3 is a flow chart of a non-limiting exemplary pre-amplification assay.

FIG. 4 is a graph of the absorbance at 405 nm in the ELISA assay described in Example 2 performed using 1:50, 1:100, or 1:500 anti-statherin antibody dilutions and 1:10, 1:50, and 1:100 saliva diluted in 50 mM bicarbonate buffer.

FIG. 5 is a graph of the mean absorbance at 405 nm of each mixed saliva and urine sample with the mean blank absorbance at 405 nm subtracted (Mean—Blank Mean).

FIG. 6 is an absorbance spectrum of synthetic urine.

FIG. 7 is an absorbance spectrum of synthetic urine with added drug and drug metabolites.

FIG. 8 is an absorbance spectrum of a urine sample originating from a first subject.

FIG. 9 is an absorbance spectrum of a urine sample originating from a second subject.

FIG. 10 is an absorbance spectrum of a urine sample originating from a third subject.

FIG. 11 is an absorbance spectrum of a urine sample originating from a fourth subject.

FIG. 12 is a graph showing the OD240 levels of serial 2-fold dilutions (S1, S2, S3, and S4) of a urine sample originating from a human subject in either synthetic urine (SU) or water.

FIG. 13 is a graph showing the OD280 levels of serial 2-fold dilutions (S1, S2, S3, and S4) of a urine sample originating from a human subject in either synthetic urine (SU) or water.

### DETAILED DESCRIPTION

Provided herein are methods of determining whether a urine sample comprises, consists essentially of, or consists of synthetic urine and methods of matching a urine sample to a subject. Also provided are methods of amplifying genomic DNA (e.g., genomic DNA isolated from mammalian cells enriched from a urine sample) and kits that can be used to perform any of the methods described herein. As can be appreciated in the art, the various aspects described below can be used in any combination without limitation.

Subjects

In any of the methods described herein, the subject has not been diagnosed as having an illegal or controlled substance addiction. In some embodiments of any of the methods described herein, the subject has been identified as having an illegal or controlled substance addiction (e.g., a subject that has already undergone treatment (e.g., successful or unsuccessful treatment) for his or her illegal or controlled substance addiction). In some embodiments of any of the methods described herein, the subject is being treated on an outpatient basis for an illegal or controlled substance addiction. In some embodiments, the subject is receiving inpatient treatment for his or her illegal or controlled substance addiction.

In some embodiments, the subject is a female (e.g., a pregnant female). In some embodiments, the subject is a male. For example, a subject in any of the methods described herein can be a child, an adolescent, a teenager, or an adult (a subject that greater than 18 years old, e.g., greater than 20 years old, greater than 25 years old, greater than 30 years old, greater than 35 years old, greater than 40 years old, greater than 45 years old, greater than 50 years old, greater than 55 years old, greater than 60 years old, greater than 65 years old, greater than 70 years old, greater than 75 years old, greater than 80 years old, greater than 90 years old, or greater than 100 years old). In any of the methods described herein, the subject may employed by the military, may be a truck driver, a train engineer, a pilot, a medical professional (e.g., a physician, nurse, nurse's assistant, or a physician's assistant), or a pharmacist. In any of the methods described herein, the subject has a family history of illegal or controlled substance addiction. In any of the methods described herein, the subject can be identified as previously submitting a urine sample comprising, consisting essentially of, or consisting of synthetic urine, a diluted urine sample, a urine sample originating from another subject, or an adulterated sample.

### Urine Samples

The methods described herein can include a step of providing a urine sample from a subject. In some examples, the methods described herein can further include a step of obtaining a urine sample from a subject. A urine sample is typically obtained using unwitnessed urine sample collection. As described herein, a urine sample can be a urine sample obtained from the subject, a urine sample comprising another subject's urine (e.g., a friend's urine, a spouse's urine, or a non-human mammal's urine), a urine sample comprising, consisting essentially of, or consisting of synthetic urine, or a diluted urine sample (e.g., diluted with water). For example, a urine sample in the methods described herein can include synthetic urine and one or more of hair, eyelashes, skin cells, saliva, semen, tears, mucus (e.g., eye or nose mucus), phlegm, or buccal cells.

A urine sample can have a volume of at least 1 mL (e.g., at least 2 mL, at least 3 mL, at least 4 mL, at least 5 mL, at least 6 mL, at least 7 mL, at least 8 mL, at least 9 mL, at least 10 mL, at least 12 mL, at least 14 mL, at least 16 mL, at least 18 mL, at least 20 mL, at least 22 mL, at least 24 mL, at least 26 mL, at least 28 mL, or at least 30 mL). For example, a urine sample can have a volume of between about 1 mL and about 30 mL, between about 5 mL and about 30 mL, between about 10 mL and about 30 mL, or between about 15 mL and about 30 mL. For example, a urine sample from a female subject can have a volume of at least 1 mL (e.g., at least 2 mL, at least 3 mL, at least 4 mL, at least 5 mL, at least 6 mL, at least 7 mL, at least 8 mL, at least 9 mL, at least 10 mL, or at least 15 mL). For example, a urine sample from a male subject can have a volume of at least 10 mL, at least 15 mL, at least 20 mL, at least 25 mL, at least 30 mL, at least 35 mL, at least 40 mL, or at least 50 mL.

In some examples of any of the methods described herein, the urine sample can be stored, e.g., for at least 1 hour (e.g., at least 6 hours, at least 12 hours, at least 1 day, at least 2 days, at least 3 days, at least 4 days, at least 5 days, at least 6 days, or at least 7 days) at a temperature below 25° C. (e.g., at about 15° C., at about 10° C., at about 4° C., at about 0° C., at about −20° C., at about −40° C., at about −80° C., at about −86° C., or at about −196° C.) prior to enriching the urine sample for mammalian cells, if present.

### Enrichment of a Urine Sample for Cells

The methods described herein include a step of enriching a urine sample (e.g., any of the urine samples described herein) for mammalian (e.g., human) cells (if present). A urine sample can be enriched for mammalian cells using a variety of different methods known in the art. For example, a urine sample can be centrifuged (e.g., ultracentrifuged) to pellet the mammalian cells present (if any) in the urine sample, the supernatant that is substantially free of mammalian cells aspirated or removed, and the resulting pellet optionally resuspended in a small volume of a buffer (e.g., a physiologically acceptable buffer or a cell lysis buffer, e.g., as the first step in the isolation of genomic DNA from the enriched sample). In another example, a container holding a urine sample can be allowed to rest (without agitation), the supernatant that is substantially free of mammalian cells is aspirated from the container at a position that is opposite of the gravitational bottom of the container, and the resulting pellet containing mammalian cells (if present) is optionally resuspended in a small volume of buffer (e.g., a physiologically acceptable buffer or a cell lysis buffer, e.g., as the first step in the isolation of genomic DNA from the enriched sample).

In another example, a urine sample can be enriched for mammalian cells by contacting the sample with a bead (e.g., a magnetic bead) coated with an antibody that specifically binds to mammalian cells (if present) in the urine sample. As is known in the art, the bound mammalian cells can be recovered from the bead in a small volume of buffer to yield an enriched sample that contains mammalian cells (if any) present in the urine sample. Similar beads that are covered with a fluorophore-labeled antibody that specifically binds to mammalian cells (if present) in the urine sample can be used to enrich any mammalian cells present in a urine sample through the use of fluorescence assisted cell sorting (FACS). Additional methods for enriching a urine sample for mammalian cells (if present) include the use of microfluidics. Such microfluidic methods are well known in the art (see, e.g., the methods described in Sethu et al., Anal. Chem. 78:5453-5461, 2006).

Isolating Genomic DNA from Enriched Samples

The methods provided herein further include a step of isolating any genomic DNA from the enriched sample to form an isolated genomic DNA test sample. A variety of methods for isolating genomic DNA from a sample (e.g., a sample containing mammalian cells enriched from a urine sample (if present)) are well-known in the art. For example, a number of commercially available kits can be used to isolate genomic DNA from a sample containing mammalian cells (e.g., any of the enriched samples described herein). Non-limiting examples of commercially available kits for the isolation of genomic DNA from a sample containing mammalian cells include: Genomic DNA Isolation Kit (Norgen Biotek Corp., Ontario, Canada), QIAmp DNA FFPE (Qiagen), QIAsymphony DSP DNA kits (Qiagen), REPLI-g Mini Kit (Qiagen), Generation Capture Plate Kit (Qiagen), Gentra Puregene Buccal Cell Kit (Qiagen), QI Amp 96 DNA Blood Kit (Qiagen), QIAmp DNA Mini kit (Qiagen), Biosprint 15 DNA Bloot Kit (Qiagen), Biosprint 96 DNA Blood Kit (Qiagen), MagAttract DNA Mini M48 Kit (Qiagen), QIAmp 96 DNA Swab BioRobot Kit, QIAmp DNA Blood BioRobot 9604 Kit (Qiagen), QIAmp DNA Investigator Kit (Qiagen), QIAmp DNA Micro Kit, ChargeSwitch® gDNA Normalized Buccal Cell Kits (Life Technologies), ChargeSwitch® gDNA Buccal Cell Kits (Life Technologies), Xtreme DNA Isolation Kit (Isohelix; Harrietsham, Kent, UK), DDK DNA Isolation Kit (Isohelix), XtraClean DNA kit (Isohelix), and EzWay Buccal Swab DNA Isolation Kit (KOMABIOTECH, Seoul, Korea). Genomic DNA can be isolated from a sample (e.g., any of the enriched samples described herein) using these and other commercially available genomic DNA isolation kits by following the manufacturer's instructions.

An exemplary method for isolating genomic DNA from an enriched sample (e.g., any of the urine samples enriched for mammalian cells described herein) include the steps of lysing the mammalian cells present (if any) in the enriched sample, precipitating proteins in the lysate, removing the supernatant, precipitating genomic DNA out of the supernatant, washing the genomic DNA pellet with ethanol, and rehydrating the genomic DNA pellet in a pharmaceutically acceptable buffer (e.g., sterile or filtered water, or a buffered solution).

### Control Samples

The methods described herein further include forming a control sample. A control sample can be formed, e.g., by adding to the isolated genomic DNA test sample (e.g., any of the isolated genomic DNA test samples described herein) a control DNA (e.g., any of the control DNAs described herein). In another embodiment, a control sample can be formed, e.g., by adding the control DNA to the enriched sample (e.g., any of the enriched samples described herein) and then isolating the DNA (e.g., using any of the methods described herein or known in the art) to form a control sample.

A control DNA can be an isolated contiguous DNA sequence that is not found in the genome of the subject (e.g., a human). A control DNA can also be a contiguous DNA sequence that is not found in a mammalian cell (e.g., an isolated contiguous DNA sequence that is not found in a human cell). For example, a control DNA can also be a DNA sequence that is not found in the genome of a bacterium (e.g., a Gram positive bacterium, a Gram negative bacterium, and a mycobacterium). A control DNA can be an isolated contiguous DNA sequence from a plant genome (e.g., spinach genome, Amborella trichopeda genome, Beta vulgaris genome, Solanum lycopersicum genome, potato genome, Mimulus guttatus genome, Vitis vinifera genome, Eucalyptus grandis genome, Populus trichocarpa genome, Linum usitatissimum genome, Manihot esculenta genome, Hevea brasiliensis genome, Betula nana genome, Cucumis sativus genome, Cucumis melo genome, Citrullus lanatus genome, Fragaria vesca genome, Malus x domestica genome, Pyrus bretschneideri genome, Cannibis sativa genome, Prunus persica, Medicago truncatula genome, Cicer arietinum genome, Glycine max genome, Cajanus cajan genome, Phaseolus vulgaris genome, Gossypium raimonddi genome, Theobroma cacao genome, Azadirachta indica genome, Citrus sinensis genome, Citrus clementina genome, Carica papaya genome, Arabidopsis thaliana genome, Arabidopsis lyrata genome, Brassica rapa genome, Capsella rubella genome, Thellungiella parvula genome, Thellungiella salsuginea genome, Thellungiella halophila genome, Phoenix dactylifera genome, Musa acuminata genome, Oryza sativa genome, Brachypodium distachyon genome, Hordeum vulgare genome, and Zea mays genome), a reptile genome (e.g., Anolis carolinensis genome), or an avian genome (e.g., Taeniopygia guttata genome, budgerigar genome, and hummingbird genome). A control DNA can be, e.g., isolated genomic DNA, a sequence of contiguous nucleotides that includes a sequence encoding a protein, a sequence that contains a cDNA sequence, a cDNA sequence, a fragment of a gene encoding a protein, or a fragment of a cDNA. A control DNA can be, e.g., a spinach chloroplast ATP synthase gamma-subunit (AtpC) gene or fragment thereof, AtpC cDNA or a fragment thereof, or a sequence containing the AtpC cDNA. For example, a control DNA containing the AtpC gene can be detected using forward and reverse primers, e.g., SEQ ID NO: 36 and SEQ ID NO: 37, respectively, in a PCR assay (e.g., a real-time PCR assay). In some embodiments where the control DNA is not genomic DNA, a control DNA that is double stranded can have a length of at least 250 base pairs, at least 300 base pairs, at least 500 base pairs, at least 1000 base pairs, at least 1500 base pairs, at least 2000 base pairs, at least 3000 base pairs, at least 4000 base pairs, or at least 5000 base pairs. A control DNA that is not genomic DNA and that is single stranded can have a length of at least 250 nucleotides, at least 300 nucleotides, at least 500 nucleotides, at least 1000 nucleotides, at least 1500 nucleotides, at least 2000 nucleotides, at least 3000 nucleotides, at least 4000 nucleotides, or at least 5000 nucleotides.

Additional control DNA can be identified using the NCBI website. Specifically, by using a searching and comparison tool (BLAST software), one skilled in the art can identify a contiguous sequence of nucleotides that is not present in the subject's genome (e.g., not present in the human genome).

### Assays to Determine the Presence of Genomic DNA

Some of the methods described herein include a step of performing an assay to determine the presence of genomic DNA in the isolated genomic DNA test sample or the control sample. A variety of assays for detecting the presence of genomic DNA are known in the art (and can be used to perform this step). For example, the detection of genomic DNA can include detection of the presence of one or more unique sequences found in genomic DNA (e.g., human genomic DNA) (e.g., satellite DNA sequences present in centromeres or heterochromatin, minisatellite sequences, microsatellite sequences, the sequence of a transposable element, a telomere sequence, a specific sequence (e.g., 250 base pairs to about 300 base pairs) containing one or more SNPs, or a specific sequence encoding a gene). Detection can be performed using labeled probes (e.g., fluorophore-, radioisotope-, enzyme-, quencher-, and enzyme-labeled probes), e.g., by hybridizing labeled probes to the genomic DNA present in the isolated genomic DNA sample or the control sample (e.g., in an electrophoretic gel) or hybridizing the labeled probes to the products of a PCR assay (e.g., a real-time PCR assay) or an assay that includes a PCR assay that utilized genomic DNA in the isolated genomic DNA test sample or the control sample as the template. Non-limiting examples of methods that can be used to generate probes include nick translation, random oligo primed synthesis, and end labeling.

As is well-known in the art, the step of detecting genomic DNA can include a step of amplifying any genomic DNA present in the isolated genomic DNA test sample or the control sample (or any fragment thereof).

In some examples, the determination of the presence of genomic DNA comprises performing an assay to determine the presence of at least one (e.g., at least two, at least three, at least four, at least five, at least six, at least seven, at least 8, at least 9, at least 10, at least 11, at least 12, at least 13, at least 14, at least 15, at least 15, at least 15, 1, 2, 3, 4, 5, 6, 7, 8, 9, 10, 11, 12, 13, 14, 15, or 16) single nucleotide polymorphisms (SNP) in the isolated genomic DNA test sample or the control sample containing genomic DNA from the enriched sample. In some embodiments, the at least one SNP (e.g., at least three SNPs) has a minor allele frequency of greater than 0.4. For example, the at least one (e.g., at least two, at least three, at least four, at least five, at least six, at least seven, at least eight, at least nine, at least ten, at least eleven, at least twelve, at least thirteen, at least fourteen, two, three, four, five, six, seven, eight, nine, ten, eleven, twelve, thirteen, or fourteen) SNP can be selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some examples where the presence of at least three SNPs are determined, the at least three SNPs include at least one SNP from at least two different chromosomes. In some examples where the presence of at least six SNPs are determined, the at least six SNPs include at least one SNP from at least four different chromosomes. In some examples where the presence of at least fourteen SNPs are determined, the at least fourteen SNPs includes at least one SNP from at least 8 different chromosomes. The assay used to determine the presence of the at least one SNP (e.g., at least three SNPs) can include a PCR assay (e.g., a real-time PCR assay or any of the other assays for genotyping a SNP described herein). In some examples, the assay used to determine the presence of the at least one SNP (e.g., at least three SNPs) can include a pre-amplification step (any of the exemplary pre-amplification steps described herein).

For example, a pre-amplification step (e.g., using any of the pre-amplification steps described herein) can include the use of one or more (e.g., one, two, three, four, five, six, seven, eight, nine, ten, eleven, twelve, thirteen, or fourteen) pairs of pre-amplification reverse and forward primers selected from the group of: SEQ ID NO: 2 and SEQ ID NO: 3, respectively; SEQ ID NO: 4 and SEQ ID NO: 5, respectively; SEQ ID NO: 6 and SEQ ID NO: 7, respectively; SEQ ID NO: 8 and SEQ ID NO: 9, respectively, SEQ ID NO: 10 and SEQ ID NO: 11, respectively; SEQ ID NO: 12 and SEQ ID NO: 13, respectively; SEQ ID NO: 14 and SEQ ID NO: 15, respectively; SEQ ID NO: 16 and SEQ ID NO: 17, respectively; SEQ ID NO: 18 and SEQ ID NO: 19, respectively; SEQ ID NO: 20 and SEQ ID NO: 21, respectively; SEQ ID NO: 22 and SEQ ID NO: 23, respectively; SEQ ID NO: 24 and SEQ ID NO: 25, respectively; SEQ ID NO: 26 and SEQ ID NO: 27, respectively; and SEQ ID NO: 28 and SEQ ID NO: 29, respectively. Additional exemplary aspects of this pre-amplification step are described below.

The presence of DNA in a sample can also be detected using a number of other well-known biochemical techniques such as, but not limited to, mass spectrometry, UV absorbance, lab-on-a-chip, microfluidics, gene chip, intercalating dyes (e.g., ethidium bromide), gel electrophoresis, Southern blotting, restriction digestion and electrophoresis, and sequencing (e.g., using any of the wide variety of sequencing methods described herein or known in the art).

An assay to determine the presence of genomic DNA in a urine sample and/or an assay to determine the presence of a control DNA (described below) in a control sample can be performed at the same time, substantially the same time, or during an overlapping time period as one or more of: an assay to determine the absorbance at 280 nm (and optionally the absorbance at 240 nm) in the urine sample (e.g., using an aliquot of the same urine sample), an assay to determine the level(s) of one or more drugs and/or one or more drug metabolites in the urine sample (e.g., using an aliquot of the same urine sample), and an assay to determine the genotype of at least one SNP in the isolated genomic DNA test sample or the control sample (e.g., using an aliquot of the same urine sample) is performed.

### Assays to Determine the Presence of Control DNA

A variety of assays are known in the art for determining the presence of control DNA in the control sample. For example, the presence of control DNA in the control sample can be detected by hybridizing a labeled probe (e.g., a fluorophore-, radioisotope-, enzyme-, quencher-, or enzyme-labeled probe) that specifically hybridizes with the control DNA.

As the sequence of the control DNA is known, a PCR assay (e.g., real-time PCR) using reverse and forward primers that specifically bind to the control DNA can be used to amplify and/or detect the control DNA. For example, when the control DNA is the spinach AtpC gene, the control DNA can be detected using a PCR assay (e.g., a real-time PCR assay) using the forward and reverse primers of SEQ ID NO: 36 and SEQ ID NO: 37, respectively. Methods for designing suitable forward and reverse primers for detecting a control DNA in the control sample are well known in the art. In addition, a number of vendors provide software tools on their websites that design suitable primers to amplify a desired target sequence (e.g., a control DNA).

### Assays to Determine the Genotype of SNPs

Some of the methods provided herein include a step of performing an assay to determine the genotype of at least one (e.g., at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) SNPs in the isolated genomic DNA test sample or the control sample. In some examples where the genotype of at least two (e.g., at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) SNPs are determined, the at least two SNPs include at least one SNP from at least two different chromosomes. In some examples where the genotype of at least three (e.g., at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) SNPs are determined, the at least three SNPs include at least one SNP from at least three different chromosomes. In some examples where the genotype of at least four (e.g., at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) SNPs are determined, the at least four SNPs include at least one SNP from at least 4 different chromosomes. In some examples where the genotype of at least six (e.g., at least seven, at least eight, at least nine, at least ten, at least eleven, at least twelve, at least thirteen, at least fourteen, at least fifteen, at least sixteen, at least seventeen, at least eighteen, at least nineteen, or at least twenty) SNPs are determined, the at least six SNPs include at least one SNP from at least six different chromosomes. In some examples where the genotype of at least eight (e.g., at least nine, at least ten, at least eleven, at least twelve, at least thirteen, at least fourteen, at least fifteen, at least sixteen, at least seventeen, at least eighteen, at least nineteen, or at least twenty) SNPs are determined, the at least eight SNPs include at least one SNP from at least eight different chromosomes. In some examples, where the subject is a genetic male, the at least one SNP includes at least a SNP (e.g., two SNPs) located on a Y chromosome.

In some embodiments, the at least one SNP (e.g., at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty) has a minor allele frequency of >0.4. For example, the at least one SNP (e.g., two, three, four, five, six, seven, eight, nine, ten, eleven, twelve, thirteen, fourteen, fifteen, sixteen, seventeen, eighteen, or nineteen SNPs) having a minor allele frequency of >0.4 is selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some examples, the at least one SNP (e.g., two, three, four, five, six, seven, eight, nine, ten, eleven, twelve, thirteen, or fourteen SNPs) are selected from the group of rs7520386, rs560681, rs9951171, rs1058083, rs1358856, rs214955, rs740598, rs279844, rs13218440, rs2272998, rs12997453, rs13134862, rs13182883, and rs1410059.

A variety of assays for determining the genotype of a SNP are known in the art. Non-limiting examples of such assays (which can be used in any of the methods described herein) include: dynamic allele-specific hybridization (see, e.g., Howell et al., Nature Biotechnol. 17:87-88, 1999), molecular beacon assays (see, e.g., Marras et al., “Genotyping Single Nucleotide Polymorphisms with Molecular Beacons,” In Kwok (Ed.), Single Nucleotide Polymorphisms: Methods and Protocols, Humana Press, Inc., Totowa, NJ, Vol. 212, pp. 111-128, 2003), SNP microarrays (see, e.g., Affymetrix Human SNP 5.0 GeneChip), restriction fragment length polymorphism (RFLP) (see, e.g., Ota et al., Nature Protocols 2:2857-2864, 2007), PCR-based assays (e.g., tetraprimerARMS-PCR (see, e.g., Zhang et al., Plos One 8:e62126, 2013), real-time PCR, allele-specific PCR (see, e.g., Gaudet et al., Methods Mol. Biol. 578:415-424, 2009), and TaqMan Assay SNP Genotyping (see, e.g., Woodward, Methods Mol. Biol. 1145:67-74, 2014, and TaqMan®OpenArray® Genotyping Plates from Life Technologies)), Flap endonuclease assays (also called Invader assays) (see, e.g., Olivier et al., Mutat. Res. 573:103-110, 2005), oligonucleotide ligation assays (see, e.g., Bruse et al., Biotechniques 45:559-571, 2008), single strand conformational polymorphism assays (see, e.g., Tahira et al., Human Mutat. 26:69-77, 2005), temperature gradient gel electrophoresis (see, e.g., Jones et al., “Temporal Temperature Gradient Electrophoresis for Detection of Single Nucleotide Polymorphisms,” in Single Nucleotide Polymophisms: Methods and Protocols, Volume 578, pp. 153-165, 2008) or temperature gradient capillary electrophoresis, denaturing high performance liquid chromatography (see, e.g., Yu et al., J. Clin. Pathol. 58:479-485, 2005), high-resolution melting of an amplified sequence containing the SNP (see, e.g., Wittwer et al., Clinical Chemistry 49:853-860, 2003), or sequencing (e.g., Maxam-Gilbert sequencing, chain-termination methods, shotgun sequencing, bridge PCR, and next-generation sequencing methods (e.g., massively parallel signature sequencing, polony sequencing, 454 pyrosequencing, Illumina (Solexa) sequencing, SOLiD sequencing, Ion Torrent semiconductor sequence, DNA nanoball sequencing, heliscope single molecule sequencing, and single molecule real-time sequencing). Additional details and a summary of various next-generation sequencing methods are described in Koboldt et al., Cell 155:27-38, 2013.

In some embodiments of any of the methods described herein, the genotyping of the at least one SNP (e.g., at least 6 SNPs) includes a PCR assay (e.g., a real-time PCR-assay, e.g., a real-time PCR-based SNP genotyping assay) (with or without a prior pre-amplification step (e.g., any of the pre-amplification methods described herein)). In some embodiments of any of the methods described herein the genotyping of the at least one SNP (e.g., at least 6 SNPs) is performed using TaqMan®-based sequencing (e.g., TaqMan®-based OpenArray® sequencing, e.g., high throughput TaqMan®-based Open Array® sequencing) (with or without a prior pre-amplification step (e.g., any of the pre-amplification methods described herein)). Additional methods for genotyping at least one SNP are described in the Examples. Methods for designing primers for use in the various SNP genotyping assays described herein are well-known in the art. For example, several vendors provide free software for designing forward and reverse primers for use in any of the SNP genotyping assays described herein. A forward or reverse primer for use in any of the SNP genotyping assays described herein can contain at least 10 (e.g., 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides). In some examples, a forward or reverse primer used in any of the SNP genotyping assays described herein can include a label (e.g., any of the exemplary labels described herein) or can include a contiguous tag sequence (e.g., between about 5 nucleotides and about 25 nucleotides, between about 10 nucleotides and about 25 nucleotides, between about 10 nucleotides and 20 nucleotides, between about 5 nucleotides and about 20 nucleotides) that does not hybridize to a sequence within the subject's genome (e.g., the human genome).

Non-limiting exemplary pairs of forward and reverse primers that can be used in a genotyping assay include: SEQ ID NO: 2 and SEQ ID NO: 3, respectively, to amplify a sequence containing rs13182883; SEQ ID NO: 4 and SEQ ID NO: 5, respectively, to amplify a sequence containing rs560681; SEQ ID NO: 6 and SEQ ID NO: 7, respectively, to amplify rs740598; SEQ ID NO: 8 and SEQ ID NO: 9, respectively, to amplify a sequence containing rs1358856; SEQ ID NO: 10 and SEQ ID NO: 11, respectively, to amplify a sequence containing rs9951171; SEQ ID NO: 12 and SEQ ID NO: 13, respectively, to amplify a sequence containing rs5720386; SEQ ID NO: 14 and SEQ ID NO: 15, respectively, to amplify a sequence containing rs13218440; SEQ ID NO: 16 and SEQ ID NO: 17, respectively, to amplify a sequence containing rs279844; SEQ ID NO: 18 and SEQ ID NO: 19, respectively, to amplify a sequence containing rs1058083; SEQ ID NO: 20 and SEQ ID NO: 21, respectively, to amplify a sequence containing rs2032597; SEQ ID NO: 22 and SEQ ID NO: 23, respectively, to amplify a sequence containing rs2032631; SEQ ID NO: 24 and SEQ ID NO: 25, respectively, to amplify a sequence containing rs2272998; SEQ ID NO: 26 and SEQ ID NO: 27, respectively, to amplify a sequence containing rs12997453; SEQ ID NO: 28 and SEQ ID NO: 29, respectively, to amplify a sequence containing rs214955; SEQ ID NO: 30 and SEQ ID NO: 31, respectively, to amplify a sequence containing rs13134862; and SEQ ID NO: 32 and SEQ ID NO: 33, respectively to amplify a sequence containing rs1410059. The sequence surrounding each SNP described herein (or any SNP genotyped in the methods described herein) can be found using the database of human SNPs (dbSNP) on the NCBI website (ncbi.nlm.nih.gov/projects/SNP/).

Any of the SNP genotyping assays described herein can include a pre-amplification step (e.g., any of the pre-amplification steps described herein). For example, the pre-amplification step can, e.g., include: hybridization of one or more pairs (e.g., two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, one, two, three, four, five, six, seven, eight, nine, ten, eleven, twelve, thirteen, fourteen, fifteen, sixteen, seventeen, eighteen, nineteen, or twenty pairs) of a pre-amplification forward and reverse primer, where each pair of pre-amplification forward and reverse primers is designed to amplify 100 base pairs to 500 base pairs (e.g., between about 150 base pairs to 450 base pairs, between about 200 base pairs to about 400 base pairs, between about 200 base pairs to about 350 base pairs, or between about 250 base pairs and 300 base pairs) of genomic DNA that contains one of the one or more targeted SNPs (e.g., any of the exemplary SNPs described herein), where the pre-amplification forward and reverse primers in each of the one or more pairs of pre-amplification primers contains: (i) sequence of about 10 to about 30 contiguous nucleotides (e.g., about 13 to about 30 contiguous nucleotides, about 15 to about 30 contiguous nucleotides, about 17 to about 30 contiguous nucleotides, or about 17 to about 25 contiguous nucleotides) that is complementary to a sequence in the genomic DNA and (ii) a tag sequence of about 5 to about 25 contiguous nucleotides (e.g., between about 10 and 20 contiguous nucleotides, between about 5 and about 20 contiguous nucleotides, or between about 17 and about 25 contiguous nucleotides) that is not complementary to a sequence in the genomic DNA; and amplification of the genomic DNA using the one or more pairs of pre-amplification forward and reverse primers to generate 100 base pair to 500 base pair (e.g., 250 base pair to 300 base pair products). In some examples, the pre-amplification method further includes amplification of the 100 base pair to 500 base pair (e.g., 250 base pair to 300 base pair products) using a primer that comprises a sequence of about 5 to about 25 contiguous nucleotides (e.g., between about 10 and 20 contiguous nucleotides, between about 5 and about 20 contiguous nucleotides, or between about 17 and about 25 contiguous nucleotides) of the tag sequence or complementary to the tag sequence. For example, the tag sequence can include or be SEQ ID NO: 1. The at least two pairs of pre-amplification forward and reverse primers can be, e.g., selected from the group of: SEQ ID NO: 2 and SEQ ID NO: 3, respectively; SEQ ID NO: 4 and SEQ ID NO: 5, respectively; SEQ ID NO: 6 and SEQ ID NO: 7, respectively; SEQ ID NO: 8 and SEQ ID NO: 9, respectively; SEQ ID NO: 10 and SEQ ID NO: 11, respectively; SEQ ID NO: 12 and SEQ ID NO: 13, respectively; SEQ ID NO: 14 and SEQ ID NO: 15, respectively; SEQ ID NO: 16 and SEQ ID NO: 17, respectively; SEQ ID NO: 18 and SEQ ID NO: 19, respectively; SEQ ID NO: 20 and SEQ ID NO: 21, respectively; SEQ ID NO: 22 and SEQ ID NO: 23, respectively; SEQ ID NO: 24 and SEQ ID NO: 25, respectively; SEQ ID NO: 26 and SEQ ID NO: 27, respectively; SEQ ID NO: 28 and SEQ ID NO: 29, respectively; SEQ ID NO: 30 and SEQ ID NO: 31, respectively; SEQ ID NO: 32 and SEQ ID NO: 33, respectively; and SEQ ID NO: 34 and SEQ ID NO: 35, respectively.

The SNP genotyping assay can be performed at the same time or substantially the same time as an aliquot of the same urine sample (as used to enrich mammalian cells (if present) and isolate genomic DNA (if present)) is analyzed for the presence of drug metabolites (e.g., by performing any of the exemplary drug metabolite assays described herein or known in the art).

An assay to determine the genotype of at least one SNP in the isolated genomic DNA test sample or the control sample can be performed at the same time, substantially the same time, or during an overlapping time period as one or more of: an assay to determine the presence of genomic DNA in the urine sample (e.g., using an aliquot of the same urine sample); an assay to determine the presence of a control DNA in a control sample; an assay to determine the absorbance at 280 nm (and optionally the absorbance at 240 nm) in the urine sample (e.g., using an aliquot of the same urine sample), and an assay to determine the level(s) of one or more drugs and/or one or more drug metabolites in the urine sample is performed (e.g., using an aliquot of the same urine sample).

### DNA Amplification Methods

Also provided herein are methods for amplifying DNA that include: hybridizing two or more (e.g., three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, three, four, five, six, seven, eight, nine, ten, eleven, twelve, thirteen, fourteen, fifteen, sixteen, seventeen, eighteen, nineteen, or twenty) pairs of a pre-amplification forward and reverse primers designed to amplify between about 100 base pairs to 500 base pairs (e.g., between about 150 base pairs to 450 base pairs, between about 200 base pairs to about 400 base pairs, between about 200 base pairs to about 350 base pairs, or between about 250 base pairs and 300 base pairs) of genomic DNA (e.g., genomic DNA that contains at least one SNP or site of mutation), where the pre-amplification forward and reverse primers in each of the two or more pairs contains (i) a sequence of about 10 to about 30 contiguous nucleotides (e.g., about 13 to about 30 contiguous nucleotides, about 15 to about 30 contiguous nucleotides, about 17 to about 30 contiguous nucleotides, or about 17 to about 25 contiguous nucleotides) that is complementary to a sequence in the genomic DNA and (ii) a tag sequence of about 5 to about 25 contiguous nucleotides (e.g., between about 10 and 20 contiguous nucleotides, between about 5 and about 20 contiguous nucleotides, or between about 17 and about 25 contiguous nucleotides) that is not complementary to a sequence in the genomic DNA; amplifying the genomic DNA using the at least two pairs of pre-amplification forward and reverse primers to generate 100 base pair to 500 base pair (e.g., 250 base pair to 300 base pair products); and amplifying the 250 to 300 base-pair amplification product(s) using a primer that comprises a sequence of about 5 to about 25 contiguous nucleotides (e.g., between about 10 and 20 contiguous nucleotides, between about 5 and about 20 contiguous nucleotides, or between about 17 and about 25 contiguous nucleotides) of the tag sequence or a sequence that is complementary to the tag sequence.

A tag sequence can be any contiguous sequence that is not present in the human genome. The amplification can be performed using any PCR-based assay (e.g., any of the PCR based assays described herein). Any of the amplification methods described herein can further include a step of sequencing the products or genotyping a SNP present in each product (e.g., using any of the SNP genotyping assay described herein or known in the art).

### Detecting One or More Saliva Proteins in Urine Sample

Some embodiments of any of the methods provided herein further include performing an assay to identify the presence of one or more saliva proteins (e.g., human statherin, human alpha-amylase, and human lysozyme) in the urine sample. Statherin is a unique phoshoprotein found in saliva. Human statherin is 62 amino acids in length. The human statherin protein sequence is shown below. A variety of antibodies that specifically bind to human statherin are commercially available (e.g., antibodies available from Santa Cruz Biotech, Abcam, and Acris).

Human Statherin Protein
(SEQ ID NO: 38)
mkflvfafil almvsmigad sseekflrri grfgygygpy
qpvpeqplyp qpyqpqyqqy tf

Human alpha-amylase is another protein that is present in saliva. Human alpha-amylase is 511 amino acids. The human alpha-amylase protein sequence is shown below. A variety of antibodies that specifically bind to human alpha-amylase are commercially available (e.g., antibodies available from BioVision, AbCam, Sigma-Aldrich, Novus Biologicals, and New England Biolabs).

Human Alpha-Amylase Protein
(SEQ ID NO: 39)
mkfflllfti gfcwaqyspn tqqgrtsivh lfewrwvdia
lecerylapk gfggvqvspp nenvaiynpf rpwweryqpv
syklctrsgn edefrnmvtr cnnvgvriyv davinhmcgn
avsagtsstc gsyfnpgsrd fpavpysgwd fndgkcktgs
gdienyndat qvrdcrltgl ldlalekdyv rskiaeymnh
lidigvagfr ldaskhmwpg dikaildklh nlnsnwfpag
skpfiyqevi dlggepikss dyfgngrvte fkygaklgtv
irkwngekms ylknwgegwg fvpsdralvf vdnhdnqrgh
gaggasiltf wdarlykmav gfmlahpygf trvmssyrwp
rqfqngndvn dwvgppnnng vikevtinpd ttcgndwvce
hrwrqirnmv ifrnvvdgqp ftnwydngsn qvafgrgnrg
fivinnddws fsltlqtglp agtycdvisg dkingnctgi
kiyvsddgka hfsisnsaed pfiaihaesk l

Human lysozyme is another protein that is present in saliva. Human lysozyme is 148 amino acids. The human lysoszyme protein sequence is shown below. A variety of antibodies that specifically bind to human lysozyme are commercially available (e.g., antibodies available from AbCam, Thermo Scientific, Novus Biologicals, and AbD Serotec).

Human Lysozyme
(SEQ ID NO: 40)
mkalivlglv llsvtvqgkv fercelartl krlgmdgyrg
islanwmcla kwesgyntra tnynagdrst dygifqinsr
ywcndgktpg avnachlscs allqdniada vacakrvvrd
pqgirawvaw rnrcqnrdvr qyvqgcgv

As is well-known in the art, a variety of antibody-based assays can be used to determine the presence of one or more of saliva proteins (e.g., statherin, alpha-amylase, and lysozyme) in the urine sample. Non-limiting examples of antibody-based assays include enzyme-linked immunosorbent assays, immunoblotting, protein chip, beads (e.g., magnetic beads) that are coated with an antibody, immunoelectrophoresis, and immunoprecipitation. For example, any of the exemplary antibodies that bind specifically to one of statherin, alpha-amylase, or lysozyme can be used in any of the antibody-based assays described herein or known in the art to determine the presence or level of statherin, alpha-amylase, or lysozyme in a urine sample.

Additional assays for determining the presence or level of one or more saliva proteins (e.g., statherin, alpha-amylase, and lysozyme) in a urine sample are well known in the art and include without limitation: mass spectrometry, enzyme activity assays (e.g., using a detectable substrate or product), electrophoresis, and protein sequencing.

### Determining the Absorbance of a Urine Sample

Some of the methods described herein further include performing an assay to determine the absorbance at 280 nm, and optionally also at 240 nm, of a urine sample. The absorbance at 280 nm, and optionally also at 240 nm, can be determined using a variety of different UV-Vis spectrophotometers known in the art. Non-limiting examples of spectrophotometers that can be used to determine the absorbance at 280 nm (and also optionally the absorbance at 240 nm) of a urine sample are commercially available from a number of vendors, e.g., Beckman Coulter, Inc., Agilent Technologies, Bibby Scientific Ltd., BioTek Instruments, Buck Scientific, Cecil Instruments Ltd., Eppendorf North America, JASCO, Ocean Optics, Shimadzu, Terra Universal Inc., Thermo Scientific, and Biochrom. A high throughput UV-Vis spectrophotometer (e.g., UH4150 UV-Visible-NIR Spectrophotometer from Hitachi High-Tech) can, e.g., be used to determine the absorbance at 280 nm (and optionally also the absorbance at 240 nm) of a urine sample.

Some examples of any of the methods described herein further include a step of experimentally diluting the urine sample from the subject (e.g., 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 60-fold, or 64-fold) prior to determination of the absorbance at 280 nm (and optionally also the absorbance at 240 nm). As is known in the art, a urine sample that has an absorbance at 280 nm (or optionally an absorbance at 240 nm) that exceeds an optical density of greater than 1.0, greater than 1.5, or greater than 2.0 may be diluted (e.g., in water) in order to increase the sensitivity of the measurement of the absorbance at 280 nm (and also optionally the absorbance at 240 nm) by a spectrophotometer.

Some embodiments of any of the methods described herein, further include a step of centrifuging a urine sample (or an aliquot of a urine sample or an experimentally diluted urine sample) prior to determining the absorbance at 280 nm (and also optionally the absorbance at 240 nm) in order to remove any particulate matter (e.g., mammalian cells, precipitated proteins, and/or precipitated lipids).

Some examples of the methods described herein include a step of comparing the determined absorbance at 280 nm of a urine sample to a reference 280 nm absorbance value. A reference 280 nm absorbance value can be, e.g., the absorbance at 280 nm of a control urine sample obtained from a subject (originating from a human subject, e.g., a human subject not receiving one or more illegal or controlled substances) (e.g., in instances where the tested urine sample is diluted, the control urine sample is diluted to the same extent using the same diluent), an average level of absorbance at 280 nm in control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent), a percentile cut-off value (e.g., 1% percentile value, 2% percentile value, 3% percentile value, 4% percentile value, 5%, percentile value, 6% percentile value, 7% percentile value, 8% percentile value, 9% percentile value, 10% percentile value, 11% percentile value, 12% percentile value, 13% percentile value, 14% percentile value, or 15% percentile value) of the absorbances at 280 nm in control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent), or an absorbance at 280 nm that is the lowest measured absorbance at 280 nm in a set of control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent).

For example, a reference 280 nm absorbance value can be an OD280 of 1.8, 1.75, 1.70, 1.65, 1.60, 1.55, 1.50, 1.45, 1.40, 1.35, 1.30, 1.25, 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, or 0.10. A reference 280 nm absorbance value can also be a threshold value at which a level below the threshold level indicates that the test urine sample comprises, consists essentially of, or consists of synthetic urine and/or the urine sample is diluted (e.g., in water or synthetic urine).

Some examples of the methods described herein include a step of comparing the determined absorbance at 240 nm of a urine sample to a reference 240 nm absorbance value. A reference 240 nm absorbance value can be, e.g., the absorbance at 240 nm of a control urine sample obtained from a subject (originating from a human subject, e.g., a subject not receiving one or more illegal or controlled substances) (e.g., in instances where the tested urine sample is diluted, the control urine sample is diluted to the same extent using the same diluent), an average level of absorbance at 240 nm in control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent), a percentile cut-off value (e.g., 1% percentile value, 2% percentile value, 3% percentile value, 4% percentile value, 5%, percentile value, 6% percentile value, 7% percentile value, 8% percentile value, 9% percentile value, 10% percentile value, 11% percentile value, 12% percentile value, 13% percentile value, 14% percentile value, or 15% percentile value) of the absorbances at 240 nm in control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent), or an absomace at 240 nm that is the lowest measured absorbance at 240 nm in control urine samples obtained from a subject population (each urine sample originating from a human subject in the population, e.g., a subject population not receiving one or more illegal or controlled substances) (e.g., in instances where the tested urine sample is diluted, the control urine samples are diluted to the same extent using the same diluent).

For example, a reference 240 nm absorbance value can be an OD240 of 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.5, 1.4, 1.3, 1.2, 1.1, 1.0, 0.9, 0.8, 0.7, 0.6, 0.5, 0.4, 0.3, or 0.2. A reference 240 nm absorbance can also be a threshold value at which a level below the threshold level indicates that the urine sample (the test urine sample) is diluted (e.g., in water).

An assay to determine the absorbance at 280 nm (and optionally the absorbance at 240 nm) in a urine sample can be performed at the same time, substantially the same time, or during an overlapping time period as one or more of: an assay to determine the presence of genomic DNA in the urine sample (e.g., using an aliquot of the same urine sample), an assay to determine the presence of a control DNA in a control sample, an assay to determine the level(s) of one or more drugs and/or one or more drug metabolites in the urine sample (e.g., using an aliquot of the same urine sample), and an assay to determine the genotype of at least one SNP in the isolated genomic DNA test sample or the control sample (e.g., using an aliquot of the same urine sample) is performed.

### Drugs and Drug Metabolites

Some of the methods described herein further include performing an assay to determine the level of one or more (e.g., two, three, four, five, six, or seven) drugs and/or the level one or more (e.g., two, three, four, five, six, or seven) drug metabolites (e.g., any of the exemplary drugs and/or drug metabolites described herein or known in the art) in a sample (e.g., a urine sample (e.g., a urine sample identified using any of the methods described herein as not comprising, consisting essentially of, or consisting of synthetic urine, an additional urine sample, or a urine sample identified using any of the methods described herein as originating from the subject) or a sample comprising blood, serum, hair, or plasma from a subject (e.g., a subject identified as providing a urine sample comprising, consisting essentially of, or consisting of synthetic urine, a subject identified as providing a urine sample not originating from the subject, a subject identified as providing a urine sample that is adulterated, or a subject identified as providing a diluted urine sample)).

Non-limiting examples of drugs and drug metabolites include: Δ9-tetrahydrocannabinol, Δ9-tetrahydrocannabino-11-oic acid, 11-hydroxy-Δ9-tetrahydrocannabinol, 11-nor-9-carboxy-Δ9-tetrahydrocannabinol, ethyl glucuronide, ethyl sulfate, morphine-3-glucuronide, morphine-6-glucu-ronide, amitriptyline, morphine 3,6-diglucuronide, morphine 3-ethereal sulfate, normorphine, cyclobenzaprine, norcodeine, codeine, normeperidine, norfentanyl, normorphine 6-glucoronide, 6-monoacetylmorphine, 6-monoacetylmorphine, 3-monoacetylmorphine, buprenorphine, morphine, clobazam, hydromorphone, hydrocodone, norhydrocodone, oxymorphone, normethadol, methadol, EDDP, EMDP, benzoylecgonine, ecgonine methyl ester, norcocaine, carisoprodol, p-hydroxycocaine, m-hydroxycocaine, p-hydroxybenzoylecgonine, m-hydroxybenzoylecgonine, methamphetamine, meperidine, meprobamate, amphetamine, MDMA, MDEA, MDA, 5-(glutathion-S-yl)-alpha-methyldopamine, 2,5-bis(glutathion-S-yl)-alpha-methyldopamine, free HMMA, DHMA sulfate, HMMA glucuronide, 7-aminoflunitrazepam, N-desmethylflunitrazepam, nitrazepam, N-desmethylclomipramine, N-desmethylcyclobenzaprine, doxepin, N-desmethylclobazam, desmethyldoxepin, 3-hydroxyflunitrazepam, gamma-hydroxybutyric acid, D-2-hydroxyglutaric acid, dehydronorketamine, maprotiline, imipramine, norketamine, 4-phenyl-4-(1-piperidinyl)cyclohexanol, dextrorphan, N-acetyl mescaline, ortriptyline, desipramine, 10-OH-nortriptyline, nortriptyline, tramadol, O-desmethyl-cis-tramadol, desmethyl-nortriptyline, fentanyl, phenobarbital, amylobarbitone, 3′-hydroxyamylobarbitone, alpha-hydroxy alprazolam, zopiclone, zolpidem, 7-amino-clonazepam, 4-hydroxymidazolam, loprazolam, flurazepam, flurazepam, 7-aminoflunitrazepam, midazolam, 1-hydroxymidazolam, norbuprenorphine, bromazepam, primidone, alpha-hydroxyalprazolam, 3-hydroxyflunitrazepam, estralozam, pentazocine, alprazolam, lorazepam, clonazepam, triazolam, desalkylfurazepam, flunitrazepam, propoxyphene, protriptyline, ritalinic acid, lormetazepam, alpha-hydroxytriazolam, desmethylflunitrazepam, methadone, diazepam, dothiepin, nordiazepam, oxazepam, methylphenidate, mianserin, naloxone, N-desmethylmirtazapine, mirtazapine, N-desmethyltapentadol, tapentadol, N-desmethyltrimipramine, trimipramine, metagynine, 7-hydroxymitragynine, AM2201, HU-210, JWH-018, JWH-018 5-pentanoic acid metabolite, JWH-073, JWH-073 4-butanoic acid metabolite, JWH-073 N-(3-hydroxybutyl) metabolite, JWH-200, JWH-250, temazepam, marijuana, hashish, heroin, an opiate, cocaine, an amphetamine, phentermine, pregabalin, methamphetamine, a MDMA, flunitrazepam, GHB, ketamine, PCP, Salvia divinorum, dextromethorphan, dextromorphan, LSD, mescaline, psilocybin, mephedrone, methylone, 3,4,-methylenedioxypyrovalerone (MDPV), an anabolic steroid, an inhalant, acetaminophen, hydrocodone, noroxycodone, oxycodone, tricyclic antidepressants, barbituates, and benzodiazepines.

A variety of urine drug assays and urine drug metabolite assays are commercially available. For example, urine drug metabolite assays can be purchased from American Screening Corp., Ameritox, Confirm Biosciences, Alibaba, Rapid Exams, DrugConfirm.

An assay to determine the level of one or more drugs and/or the level of one or more drug metabolites in a urine sample (e.g., any of the urine samples described herein from any subject described herein) can be performed at the same time as the detection of genomic DNA (if present) in the isolated genomic DNA sample or the control sample or at the same time the at least one SNP is genotyped (e.g., in the isolated genomic DNA sample or the control sample).

As is well known in the art, the determined level of the one or more drugs and/or the determined level of the one or more drug metabolites can be compared to reference values of the one or more drugs and/or the one or more drug metabolites (e.g., the level of the one or more drugs and/or the level of one or more drug metabolites in a subject that has not been administered a drug and/or an agent that is not metabolized into the one or more drug metabolites).

### Methods of Determining if a Urine Sample Comprises, Consists Essentially of, or Consists of Synthetic Urine by Determining the Presence of Genomic DNA

Provided herein are methods of determining if a urine sample comprises, consists essentially of, or consists of synthetic urine that include: (a) providing a urine sample (e.g., any of the urine samples described herein) from a subject (e.g., any of the subjects described herein, e.g., a human); (b) enriching the urine sample for mammalian cells, if present; isolating any genomic DNA from the enriched sample of step (b) to form an isolated genomic DNA test sample; adding to the isolated genomic DNA test sample of step (c) a control DNA to form a control sample or adding the control DNA to the enriched sample of step (b) and then isolating the DNA to form a control sample; (e) performing an assay to determine the presence of genomic DNA in the isolated genomic DNA sample of step (c) or the control sample of step (d); and (g) identifying a urine sample having no detectable level of genomic DNA and having detectable control DNA as comprising, consisting essentially of, or consisting of synthetic urine, or identifying a urine sample having a detectable level of genomic DNA and having detectable control DNA as not comprising synthetic urine.

The step of enriching the urine sample for mammalian cells, if present, can be performed using any of the exemplary methods for performing such enrichment described herein or known in the art. The step of isolating any genomic DNA from the enriched sample of step (b) can be performed using any methods for isolating genomic DNA from an enriched sample described herein or known in the art. The step of performing an assay to determine the presence of genomic DNA in the isolated genomic DNA test sample of step (c) or the control sample of step (d) can be performed using any of the exemplary methods described herein or known in the art. The step of performing an assay to determine the presence of the control DNA in the control sample of step (d) can be performed using any of the methods described herein or known in the art.

In some examples, the determination of the presence of genomic DNA comprises performing an assay to determine the presence of at least one (e.g., at least two, at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs in the isolated genomic DNA test sample of step (c) or the control sample of step (d); and a urine sample having no detectable level of the at least one SNP and having detectable control DNA is identified in step (g) as comprising, consisting essentially of, or consisting of synthetic urine, or a urine sample having a detectable level of the at least one SNP and having detectable control DNA is identified in step (g) as not comprising synthetic urine.

In some examples, the urine sample is identified in step (g) as not comprising synthetic urine. Such examples can further include performing an assy to determine the level of one or more drugs and/or the level of one or more drug metabolites (e.g., any of the exemplary drugs and/or drug metabolites described herein or known in the art) in the urine sample identified in step (g) as not comprising synthetic urine. In some embodiments, the level or one or more drugs and/or the level of one or more drug metabolites in the urine sample (e.g., another aliquot of the same starting urine sample) can be determined at the substantially the same time (or over the same time period) as the assay to determine the presence of genomic DNA in the isolated genomic DNA sample of step (c) or the control sample of step (d) is performed.

In some examples, when the urine sample is identified in step (g) as not comprising synthetic urine can also further include: (h) performing an assay to determine the genotype of at least one (e.g., at least two, at least three, at least four, at least five, at least six, at least seven, at least eight, at least nine, at least ten, at least eleven, at least twelve, at least thirteen, at least fourteen, two, three, four, five, six, seven, eight, nine, ten, eleven, twelve, thirteen, or fourteen) SNPs in the isolated genomic DNA test sample of step (b) or the control sample of step (d) (or an isolated DNA test sample of step (b) or control sample of step (d) prepared from a different aliquot of the same starting urine sample); (i) comparing the genotype of the at least one SNP in the isolated genomic DNA test sample of step (c) or the control sample of step (d) with the genotype of the at least one SNP in a control cell sample from the subject; and (j) identifying a urine sample having a detectable level of the control DNA and having the same genotype of the at least one SNP in the isolated genomic DNA test sample of step (c) or in the control sample of step (d) as the genotype of the at least one SNP in the control cell sample as originating from the subject (or when at least six SNPs are genotyped, identifying a urine sample having a detectable level of the control DNA and having the same genotype of the at least six SNPs in the isolated genomic DNA test sample of step (c) or in the control sample of step (d) as compared to the genotype of the at least six SNPs in the control cell sample, except for one or two SNPs, as originating from the subject) (or when at least ten (e.g., at least 16) SNPs are genotyped, identifying a urine sample having a detectable level of the control DNA and having the same genotype of the at least ten (e.g., at least 16) SNPs in the isolated genomic DNA test sample of step (c) or in the control sample of step (d) as compared to the genotype of the at least ten (e.g., at least 16) SNPs in the control cell sample, except for one, two, or three SNPs, as originating from the subject), or identifying a urine sample having a detectable level of the control DNA and not having the same genotype of the at least one SNP in the isolated genomic DNA test sample of (c) or the control sample of step (d) as the genotype of the at least one SNP in the control cell sample as not originating from the subject (or when at least six SNPs are genotyped, identifying a urine sample having a detectable level of the control DNA and having the same genotype at only one or two of the at least six SNPs in the isolated genomic DNA test sample of step (c) or in the control sample of step (d) as compared to the genotype of the at least six SNPs in the control cell sample, as not originating from the subject) (or when at least at least ten (e.g., at least 16) SNPs are genotyped, identifying a urine sample having a detectable level of control DNA and having the same genotype at one one, two, or three of the at least ten (e.g., at least 16) SNPs in the isolated genomic DNA test sample of step (c) or in the control sample of step (d) as compared to the genotype of the at least ten (e.g., at least 16) SNPs in the control cell sample, as not originating from the subject). The control cell sample can be any of the control cell samples described herein. Some embodiments further include obtaining a control cell sample from the subject.

In some embodiments of these methods, the at least one SNP has a minor allele frequency of >0.4. The at least one SNP, e.g., can be selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some examples, the at least two (e.g., at least three, at least four, at least five, at least six, at least seven, at least eight, at least nine, at least ten, at least eleven, at least twelve, at least thirteen, at least fourteen, two, three, four, five, six, seven, eight, nine, ten, eleven, twelve, thirteen, or fourteen) SNPs include at least one SNP from at least two (e.g., three, four, five, six, seven, eight, nine, ten, eleven, or twelve) different chromosomes.

In some examples, the assay in step (e) comprises a PCR assay (e.g., a real-time PCR assay). In some examples, the assay in step (e) includes a pre-amplification step (e.g., any of the pre-amplification steps described herein or known in the art.

In some examples, the control DNA is a plant DNA (e.g., a cDNA or gene encoding spinach chloroplast ATP synthase gamma-subunit (AtpC)). In some examples, the assay in step (f) includes a PCR assay (e.g., a real-time PCR assay). For example, when the control cDNA is AtpC the PCR assay can, e.g., utilize forward and reverse primers having the sequence of SEQ ID NO: 36 and SEQ ID NO: 37, respectively.

Some methods further include: (h) performing an assay to identify the presence of one or more saliva proteins (e.g., one or more of human statherin, human alpha-amylase, and human lysozyme) in the urine sample, and (i) identifying a urine sample having a detectable level of genomic DNA, a detectable control DNA, and a detectable level of the one or more saliva proteins (e.g., one or more of human statherin, human alpha-amylase, and human lysozyme) as being adulterated. In some embodiments, the assay in step (h) is an enzyme activity assay or an enzyme-linked immunosorbent assay.

Some examples of the methods further include recoding the identification in step (g) in the subject's clinical record (e.g., a computer readable medium). Some examples of the methods further include notifying the subject's insurance provider, employer, or potential future employer of the identification in step (g). Some examples of the methods further include notifying a pharmacist or a medical professional of the identification in step (g). Some examples of the methods further include (h) selecting a subject having a urine sample identified in step (g) as comprising, consisting essentially of, or consisting of synthetic urine, and obtaining an additional urine sample from the selected subject. In some examples, the additional urine sample is a witnessed urine test. Some embodiments further include (j) performing an assay to determine the level of one or more drugs and/or the level of one or more drug metabolites in the additional urine sample. Some examples further include: (k) identifying a subject having an elevated level of one or more drugs and/or an elevated level of one or more drug metabolites in the additional urine sample as compared to a reference level of the one or more drugs and/or a reference level of the one or more drug metabolites, where the drugs are an illegal or controlled substance and/or the drug metabolites are metabolites of an illegal or controlled substance; and (l) admitting the subject into a drug dependency program, ceasing administration of the controlled substance to the subject, or reducing the dose and/or frequency of administration of the controlled substance to the subject. In some examples, the drug dependency program includes administering to the subject in step (l) a drug replacement therapy.

Some embodiments further include (h) selecting a subject having a urine sample identified in step (g) as comprising, consisting essentially of, or consisting of synthetic urine, or a subject identified as having an adulterated urine sample, for heightened monitoring (e.g., a clinical visit at least once a month, at least once every six weeks, or at least once every two months). Some embodiments include performing heightened monitoring of the selected subject for at least 3 months (e.g., at least six months, at least one year, at least two years, or at least three years).

Some examples of these methods further include: (h) selecting a subject having a urine sample identified in step (g) as comprising, consisting essentially of, or consisting of synthetic urine; and (i) obtaining a sample comprising blood, serum, hair, or plasma from the subject, and (j) performing an assay to determine the level of one or more drugs and/or the level of one or more drug metabolites in the sample from step (i). Some embodiments further include (k) identifying the subject having an elevated level of one or more drugs and/or an elevated level of one or more drug metabolites in the sample from step (i) as compared to a reference level of the one or more drugs and/or a reference level of the one or more drug metabolites, where the drugs are an illegal or controlled substance and/or the drug metabolites are metabolites from an illegal or controlled substance; and (l) admitting the subject into a drug dependency program (e.g., a drug dependency program that includes administering to the subject in step (l) a drug replacement therapy), ceasing administration of the controlled substance to the subject, or reducing the dose and/or frequency of administration of the controlled substance to the subject.

### Methods of Detecting the Presence of Synthetic Urine or a Diluted Urine Sample Using Spectrophotometry

Also provided herein are methods of determining if a urine sample comprises synthetic urine and/or is diluted that include: (a) providing a urine sample (e.g., any of the urine samples described herein) from a subject (e.g., any of the subjects described herein, e.g., a human); (b) detecting the absorbance at 280 nm of the urine sample; and (c) identifying a urine sample having an absorbance at 280 nm that is less than a reference 280 nm absorbance value as comprising, consisting essentially of, or consisting of synthetic urine and/or being diluted, or identifying a urine sample having an absorbance at 280 nm that is equal to or greater than the reference 280 nm absorbance value (e.g., any of the exemplary reference 280 absorbance values described herein) as not comprising synthetic urine and not being diluted. Some embodiments further include after step (a) and before step (b), centrifuging the urine sample to remove particulate material (e.g., mammalian cells, precipitated proteins, and/or precipitated lipids). Methods for centrifuging a sample to remove particulate material are well-known in the art. Any of the exemplary methods described herein for determining the absorbance at 280 nm (and optionally the absorbance at 240 nm) in a urine sample can be used in these methods. Additional methods for determining the absorbance at 280 nm (and optionally the absorbance at 240 nm) in a liquid sample are known in the art and can be used to determine the absorbance at 280 (and optionally the absorbance at 240 nm) in a urine sample in any of the methods described herein. A urine sample can, optionally, be diluted prior to determining the absorbance at 280 nm (and optionally the absorbance at 240 nm). Non-limiting dilution factors and dilution buffers that can be used to dilute a urine sample are described herein.

Some embodiments of these methods further include (d) determining the absorbance at 240 nm of the urine sample and (e) further identifying a urine sample having an absorbance at 280 nm that is less than a reference 280 nm absorbance value (e.g., any of the exemplary reference 280 nm absorbance values described herein) and an absorbance at 240 nm that is less than a reference 240 nm absorbance value (e.g., any of the exemplary reference 240 nm absorbance values described herein) as being diluted (e.g., in water).

Any of the exemplary reference 280 nm absorbance values described herein can be used in any of the methods described herein. Any of the exemplary reference 240 nm absorbance values described herein can be used in any of the methods described herein.

In some examples of these methods, the urine sample is identified in step (c) as not comprising synthetic urine and not being diluted. Some such examples further include performing an assay (e.g., at substantially the same time the absorbance at 280 nm (and optionally the absorbance at 240 nm) is determined in an aliquot of the same urine sample) to determine the level of one or more drugs and/or one or more drug metabolites in the urine sample identified as not comprising, consisting essentially of, or consisting of synthetic urine and not being diluted.

In some examples of these methods, a sample identified as comprising, consisting essentially of, or consisting of synthetic urine is used in a further method described herein to confirm whether the urine sample comprises, consists essentially of, or consists of synthetic urine (e.g., any of the methods that include the detection of the presence of genomic DNA in the urine sample described herein). Such a further method can be performed at substantially the same time using an aliquot of the same urine sample used in the methods described in this section (e.g., methods that include determining the absorbance at 280 nm (and optionally the absorbance at 240 nm) in an aliquot of the same urine sample).

Some examples, where a sample identified as not comprising synthetic urine and not being diluted, further include: (d) enriching the urine sample (or an aliquot of the urine sample) for mammalian cells, if present; (e) isolating any genomic DNA from the enriched sample of step (d) to form an isolated genomic DNA test sample; (f) adding to the isolated genomic DNA test sample of step (e) a control DNA to form a control sample or adding the control DNA to the enriched sample of step (d) and then isolating the DNA to form a control sample; (g) performing an assay to determine the genotype of at least two (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) single nucleotide polymorphisms (SNPs) in the isolated genomic DNA test sample of step (e) or the control sample of step (f); (h) comparing the genotype of the at least 2 SNPs in the isolated genomic DNA test sample of step (e) or the control sample of step (f) with the genotype of the at least 2 SNPs in a control cell sample from the subject; (i) performing an assay to determine the presence of the control DNA in the control sample of step (f); and (j) identifying a urine sample having a detectable level of the control DNA and having the same genotype of the at least 2 SNPs in the isolated genomic DNA test sample of step (e) or the control sample of step (f) as the genotype of the at least 2 SNPs in the control cell sample as originating from the subject (or when at least six SNPs are genotyped, identifying a urine sample having a detectable level of the control DNA and having the same genotype of the at least six SNPs in the isolated genomic DNA test sample of step (e) or in the control sample of step (f) as compared to the genotype of the at least six SNPs in the control cell sample, except for one or two SNPs, as originating from the subject) (or when at least ten (at least 16) SNPs are genotyped, identifying a urine sample having a detectable level of the control DNA and having the same genotype of the at least ten (at least 16) SNPs in the isolated genomic DNA test sample of step (e) or in the control sample of step (f) as compared to the genotype of the at least ten (e.g., at least 16) SNPs in the control cell sample, except for one, two, or three SNPs, as originating from the subject); or identifying a urine sample having a detectable level of the control DNA and not having the same genotype of the at least 2 SNPs in the isolated genomic DNA test sample of step (e) or the control sample of step (f) as the genotype of the at least 6 SNPs in the control cell sample as not originating from the subject (or when at least six SNPs are genotyped, identifying a urine sample having a detectable level of the control DNA and having the same genotype at only one or two of the at least six SNPs in the isolated genomic DNA test sample of step (e) or in the control sample of step (f) as compared to the genotype of the at least six SNPs in the control cell sample, as not originating from the subject) (or when at least ten (e.g., at least 16) SNPs are genotyped, identifying a urine sample having a detectable level of the control DNA and having the same genotype at only one, two, or three of the at least ten (e.g., at least 16) in the isolated genomic DNA test sample of step (e) or in the control sample of step (f) as compared to the genotype of the at least ten (e.g., at least 16) SNPs in the control cell sample, as not originating from the subject).

A control cell sample can be any of the control cell samples described herein or known in the art. Some of the methods described herein further include a step of obtaining a control cell sample from the subject. In some examples, the control cell sample is a buccal cell sample. Some examples of the methods provided herein further include performing an assay to determine the genotype of the at least two SNPs in the control cell sample (e.g., using any of the exemplary SNP genotyping assays described herein or known in the art).

The step of enriching the urine sample for mammalian cells, if present, can be performed using any of the exemplary methods for performing such enrichment described herein or known in the art. The step of isolating any genomic DNA from the enriched sample of step (b) can be performed using any methods for isolating genomic DNA from an enriched sample described herein or known in the art. The step of performing an assay to determine the genotype of the at least two SNPs in the genomic DNA sample of step (e) can be performed using any of the exemplary SNP genotyping assays or methods described herein or known in the art. The step of performing an assay to determine the presence of the control DNA in the control sample of step (g) can be performed using any of the methods described herein or known in the art.

In some examples, the urine sample is identified in step (h) as originating from the subject. Such examples can further include performing an assay to determine the level of one or more drugs and/or the level of one or more drug metabolites (e.g., any of the exemplary drugs and/or drug metabolites described herein or known in the art) in the urine sample (or an aliquot of the same starting urine sample) identified in step (h) as originating from the subject.

In some embodiments of these methods, the at least two SNPs (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen or fourteen SNPs) have a minor allele frequency of >0.4. The at least two SNPs (e.g., at least three, at least four, at least five, at least six, at least seven, at least eight, at least nine, at least ten, at least twelve, at least thirteen, at least fourteen, at least fifteen, at least sixteen, at least seventeen, at least eighteen, at least nineteen, three, four, five, six, seven, eight, nine, ten, eleven, twelve, thirteen, fourteen, fifteen, sixteen seventeen, eighteen, or nineteen SNPs) genotyped, e.g., can be selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. The at least two SNPs (e.g., at least three, at least four, at least five, at least six, at least seven, at least eight, at least nine, at least ten, at least twelve, at least thirteen, at least fourteen, three, four, five, six, seven, eight, nine, ten, eleven, twelve, thirteen, or fourteen SNPs) genotyped, e.g., can be selected from the group of rs7520386, rs560681, rs9951171, rs1058083, rs1358856, rs214955, rs740598, rs279844, rs13218440, rs2272998, rs12997453, rs13134862, rs13182883, and rs1410059. In some examples, where the subject is a genetic male, at least one of the SNPs in step (e) is located on a Y chromosome, and no detectable level of the at least one of the SNPs located on the Y chromosome further identifies the urine sample as not originating from the subject. In some examples, the at least two (e.g., at least two, at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs include at least one SNP from at least two (e.g., three, four, five, six, seven, eight, nine, ten, eleven, or twelve) different chromosomes.

In some examples, the assay in step (g) comprises a PCR assay (e.g., a real-time PCR assay). In some examples, the assay in step (g) includes a pre-amplification step (e.g., any of the pre-amplification steps described herein or known in the art.

In some examples, the control DNA is a plant DNA (e.g., a cDNA or gene encoding spinach chloroplast ATP synthase gamma-subunit (AtpC)). In some examples, the assay in step (i) includes a PCR assay (e.g., a real-time PCR assay). For example, when the control cDNA is AtpC the PCR assay can, e.g., utilize forward and reverse primers having the sequence of SEQ ID NO: 36 and SEQ ID NO: 37, respectively.

Some methods further include: (k) performing an assay to identify the presence of one or more saliva proteins (e.g., one or more of human statherin, human alpha-amylase, and human lysozyme) in the urine sample, and (l) identifying a urine sample having a detectable level of genomic DNA, a detectable control DNA, and a detectable level of the one or more saliva proteins (e.g., one or more of human statherin, human alpha-amylase, and human lysozyme) as being adulterated. In some embodiments, the assay in step (k) is an enzyme activity assay or an enzyme-linked immunosorbent assay.

Some embodiments further include selecting a subject having a urine sample identified in step (j) as not originating from the subject, or a subject identified as having an adulterated urine sample, for heightened monitoring (e.g., a clinical visit at least once a month, at least once every six weeks, or at least once every two months). Some embodiments further include selecting a subject having a urine sample identified in step (c) as comprising, consisting essentially of, or consisting of synthetic urine and/or being diluted, for heightened monitoring (e.g., a clinical visit at least once a month, at least once every six weeks, or at least once every two months). Some embodiments further include selecting a subject having a urine sample identified in step (e) as being diluted, for heightened monitoring (e.g., a clinical visit at least once a month, at least once every six weeks, or at least once every two months). Some embodiments include performing heightened monitoring of the selected subject (e.g., any of the selected subjects described herein) for at least 3 months (e.g., at least six months, at least one year, at least two years, or at least three years).

Some embodiments of any of these methods further include recording the identification in step (c), the identification in step (e), and/or the identification in step (j) in the subject's medical record (e.g., a computer readable medium). Some embodiments of any of these methods further include notifying the subject's insurance provider, employer, or potential future employer of the identification in step (c), the identification in step (e), and/or the identification in step j). Some embodiments of any of these methods further include notifying a pharmacist or a medical professional (e.g., any of the exemplary medical professionals described herein) of the identification in step (c), the identification in step (e), and/or the identification in step j).

Some embodiments of any of these methods further include (d) selecting a subject having a urine sample identified in step (c) as comprising, consisting essentially of, or consisting of synthetic urine and/or being diluted; and (e) obtaining an additional urine sample from the subject. Some embodiments of any of these methods further include (f) selecting a subject having a urine sample identified in step (e) as being diluted, and (g) obtaining an additional urine sample from the subject. Some embodiments of any of these methods further include (k) selecting a subject having a urine sample identified in step (j) as not originating from the subject; and (l) obtaining an additional urine sample from the selected subject. In some examples, the additional urine sample is obtained through a witnessed urine test. Some embodiments of any of these methods further include performing an assay to determing the level of one or more drugs and/or one or more drug metabolites in the additional urine sample. Some embodiments of any of these methods further include identifying a subject having an elevated level of one or more drugs and/or an elevated level of one or more drug metabolites in the additional urine sample as compared to a reference level of the one or more drugs and/or a reference level of one or more drug metabolites, wherein the drugs are an illegal or controlled substance and/or the drug metabolites are metabolites of an illegal or controlled substance; and admitting the identified subject into a drug dependency program (e.g., a drug dependency program that includes administering to the admitted subject a drug replacement therapy), ceasing administration of the controlled substance to the identified subject, or reducing the dose and/or frequency of administration of the controlled substance to the identified subject.

Some embodiments of any of these methods further include (d) selecting a subject having a urine sample identified in step (c) as comprising, consisting essentially of, or consisting of synthetic urine and/or being diluted, (e) obtaining an additional sample comprising blood, serum, hair, or plasma from the subject, and (f) performing an assay to determine the level of one or more drugs and/or the level of one or more drug metabolites in the additional sample from step (e). Some embodiments of any of these methods further include (f) selecting a subject having a urine sample identified as being diluted, (g) obtaining an additional sample comprising blood, serum, hair, or plasma from the subject; and (h) performing an assay to determine the level of one or more drugs and/or the level of one or more drug metabolites in the additional sample from step (g). Some embodiments of any of these methods further include (k) selecting a subject having a urine sample identified in step (j) as not originating from the subject, (l) obtaining an additional sample comprising blood, serum, hair, or plasma from the subject; and (m) performing an assay to determine the level of one or more drugs and/or the level of one or more drug metabolites in the additional sample from step (l). Some embodiments of any of these methods further include identifying a subject having an elevated level of one or more drugs and/or an elevated level of one or more drug metabolites in the additional sample as compared to a reference level of the one or more drugs and/or a reference level of the one or more drug metabolites, where the drugs are an illegal or controlled substance and/or the drug metabolites are metabolites of an illegal or controlled substance; and admitting the identified subject into a drug dependency program (e.g., a drug dependency program that includes administering to the admitted subject a drug replacement therapy), ceasing administration of the controlled substance to the identified subject, or reducing the dose or frequency of administration of the controlled substance to the identified subject.

Methods of Matching a Urine Sample to a Subject

Also provided herein are methods of matching a urine sample to a subject that include: (a) providing a urine sample (e.g., any of the urine samples described herein) from a subject (e.g., any of the subjects described herein, e.g., a human); (b) enriching the urine sample for mammalian cells, if present; (c) isolating any genomic DNA from the enriched sample of step (b) to form an isolated genomic DNA test sample; (d) adding to the isolated genomic DNA test sample of step (c) a control DNA to form a control sample or adding the control DNA to the enriched sample of step (b) and then isolating the DNA to form a control sample; (e) performing an assay to determine the genotype of at least two (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs in the isolated genomic DNA test sample of step (c) or the control sample of step (d); (f) comparing the genotype of the at least two SNPs in the isolated genomic DNA test sample of step (c) or the control sample of step (d) with the genotype of the at least two SNPs in a control cell sample from the subject; (g) performing an assay to determine the presence of the control DNA in the control sample of step (d); (h) identifying a urine sample having a detectable level of the control DNA and having the same genotype of the at least two SNPs in the isolated genomic DNA test sample of step (c) or the control sample of step (d) as the genotype of the at least two SNPs in the control cell sample as originating from the subject (or when at least six SNPs are genotyped, identifying a urine sample having a detectable level of the control DNA and having the same genotype of the at least six SNPs in the isolated genomic DNA test sample of step (c) or in the control sample of step (d) as compared to the genotype of the at least six SNPs in the control cell sample, except for one or two SNPs, as originating from the subject), or identifying a urine sample having a detectable level of the control DNA and not having the same genotype of the at least two SNPs in the isolated genomic DNA test sample of (c) or the control sample of step (d) as the genotype of the at least two SNPs in the control cell sample as not originating from the subject (or when at least six SNPs are genotyped, identifying a urine sample having a detectable level of the control DNA and having the same genotype at only one or two of the at least six SNPs in the isolated genomic DNA test sample of step (c) or in the control sample of step (d) as compared to the genotype of the at least six SNPs in the control cell sample, as not originating from the subject).

A control cell sample can be any of the control cell samples described herein or known in the art. Some of the methods described herein further include a step of obtaining a control cell sample from the subject. In some examples, the control cell sample is a buccal cell sample. Some examples of the methods provided herein further include performing an assay to determine the genotype of the at least two SNPs in the control cell sample (e.g., using any of the exemplary SNP genotyping assays described herein or known in the art).

The step of enriching the urine sample for mammalian cells, if present, can be performed using any of the exemplary methods for performing such enrichment described herein or known in the art. The step of isolating any genomic DNA from the enriched sample of step (b) can be performed using any methods for isolating genomic DNA from an enriched sample described herein or known in the art. The step of performing an assay to determine the genotype of the at least two SNPs in the genomic DNA sample of step (e) can be performed using any of the exemplary SNP genotyping assays or methods described herein or known in the art. The step of performing an assay to determine the presence of the control DNA in the control sample of step (g) can be performed using any of the methods described herein or known in the art.

In some examples, the urine sample is identified in step (h) as originating from the subject. Such examples can further include performing an assay to determine the level of one or more drugs and/or the level of one or more drug metabolites (e.g., any of the exemplary drugs and/or drug metabolites described herein or known in the art) in the urine sample (or an aliquot of the same starting urine sample) identified in step (h) as originating from the subject.

In some embodiments of these methods, the at least two SNPs (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen or fourteen SNPs) have a minor allele frequency of >0.4. The at least two SNPs (e.g., at least three, at least four, at least five, at least six, at least seven, at least eight, at least nine, at least ten, at least twelve, at least thirteen, at least fourteen, at least fifteen, at least sixteen, at least seventeen, at least eighteen, at least nineteen, three, four, five, six, seven, eight, nine, ten, eleven, twelve, thirteen, fourteen, fifteen, sixteen seventeen, eighteen, or nineteen SNPs) genotyped, e.g., can be selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. The at least two SNPs (e.g., at least three, at least four, at least five, at least six, at least seven, at least eight, at least nine, at least ten, at least twelve, at least thirteen, at least fourteen, three, four, five, six, seven, eight, nine, ten, eleven, twelve, thirteen, or fourteen SNPs) genotyped, e.g., can be selected from the group of: rs7520386, rs560681, rs9951171, rs1058083, rs1358856, rs214955, rs740598, rs279844, rs13218440, rs2272998, rs12997453, rs13134862, rs13182883, and rs1410059. In some examples, where the subject is a genetic male, at least one of the SNPs in step (e) is located on a Y chromosome, and no detectable level of the at least one of the SNPs located on the Y chromosome further identifies the urine sample as not originating from the subject. In some examples, the at least two (e.g., at least two, at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs include at least one SNP from at least two (e.g., three, four, five, six, seven, eight, nine, ten, eleven, or twelve) different chromosomes.

In some examples, the assay in step (e) comprises a PCR assay (e.g., a real-time PCR assay). In some examples, the assay in step (e) includes a pre-amplification step (e.g., any of the pre-amplification steps described herein or known in the art.

In some examples, the control DNA is a plant DNA (e.g., a cDNA or gene encoding spinach chloroplast ATP synthase gamma-subunit (AtpC)). In some examples, the assay in step (f) includes a PCR assay (e.g., a real-time PCR assay). For example, when the control cDNA is AtpC the PCR assay can, e.g., utilize forward and reverse primers having the sequence of SEQ ID NO: 36 and SEQ ID NO: 37, respectively.

Some methods further include: (i) performing an assay to identify the presence of one or more saliva proteins (e.g., one or more of human statherin, human alpha-amylase, and human lysozyme) in the urine sample, and (j) identifying a urine sample having a detectable level of genomic DNA, a detectable control DNA, and a detectable level of the one or more saliva proteins (e.g., one or more of human statherin, human alpha-amylase, and human lysozyme) as being adulterated. In some embodiments, the assay in step (i) is an enzyme activity assay or an enzyme-linked immunosorbent assay.

Some examples of the methods further include recoding the identification in step (h) in the subject's clinical record (e.g., a computer readable medium). Some examples of the methods further include notifying the subject's insurance provider, employer, or potential future employer of the identification in step (h). Some examples of the methods further include notifying a pharmacist or a medical professional of the identification in step (h). Some examples of the methods further include (i) selecting a subject having a urine sample identified in step (h) as not originating from the subject, and (j) obtaining an additional urine sample from the selected subject. In some examples, the additional urine sample is a witnessed urine test. Some embodiments further include (k) performing an assay to determine the level of one or more drugs and/or the level of one or more drug metabolites in the additional urine sample. Some examples further include: (l) identifying a subject having an elevated level of one or more drugs and/or an elevated level of one or more drug metabolites in the additional urine sample as compared to a reference level of the one or more drugs and/or a reference level of the one or more drug metabolites, where the drugs are an illegal or controlled substance and/or the drug metabolites are metabolites of an illegal or controlled substance; and (m) admitting the subject into a drug dependency program, ceasing administration of the controlled substance to the subject, or reducing the dose and/or frequency of administration of the controlled substance to the subject. In some examples, the drug dependency program includes administering to the subject in step (m) a drug replacement therapy.

Some embodiments further include (i) selecting a subject having a urine sample identified in step (h) as not originating from the subject, or a subject identified as having an adulterated urine sample, for heightened monitoring (e.g., a clinical visit at least once a month, at least once every six weeks, or at least once every two months). Some embodiments include performing heightened monitoring of the selected subject for at least 3 months (e.g., at least six months, at least one year, at least two years, or at least three years). Some examples of these methods further include: (i) selecting a subject having a urine sample identified in step (h) as not originating from the subject; (j) obtaining a sample comprising blood, serum, hair, or plasma from the subject, and (k) performing an assay to determine the level of one or more drugs and/or the level of one or more drug metabolites in the sample from step (j). Some embodiments further include (l) identifying the subject having an elevated level of one or more drugs and/or an elevated level of one or more drug metabolites in the sample from step (j) as compared to a reference level of the one or more drugs and/or a reference level of the one or more drug metabolites, where the drug is an illegal or controlled substance and/or the drug metabolites are metabolites from an illegal or controlled substance; and (m) admitting the subject into a drug dependency program (e.g., a drug dependency program that includes administering to the subject in step (m) a drug replacement therapy), ceasing administration of the controlled substance to the subject, or reducing the dose and/or frequency of administration of the controlled substance to the subject.

### Kits

Also provided herein are kits that consist essentially of or consist of (i) a set of at least 2 (e.g., at least three, at least four, at least five, at least six, at least seven, at least eight, at least nine, at least ten, at least eleven, at least twelve, at least thirteen, at least fourteen, at least fifteen, at least sixteen, three, four, five, six, seven, eight, nine, ten, eleven, twelve, thirteen, fourteen, fifteen, or sixteen) pairs of a pre-amplification forward and reverse primer, where each pair of forward and reverse primers is designed to amplify 100 base pairs to 500 base pairs (e.g., between about 150 base pairs to 450 base pairs, between about 200 base pairs to about 400 base pairs, between about 200 base pairs to about 350 base pairs, or between about 250 base pairs and 300 base pairs) of genomic DNA (e.g., genomic DNA that contains at least one SNP or site of mutation), where the pre-amplification forward and reverse primers in each of the two or more pairs contains (i) a sequence of about 10 to about 30 contiguous nucleotides (e.g., about 13 to about 30 contiguous nucleotides, about 15 to about 30 contiguous nucleotides, about 17 to about 30 contiguous nucleotides, or about 17 to about 25 contiguous nucleotides) that is complementary to a sequence in the genomic DNA and (ii) a tag sequence of about 5 to about 25 contiguous nucleotides (e.g., between about 10 and 20 contiguous nucleotides, between about 5 and about 20 contiguous nucleotides, or between about 17 and about 25 contiguous nucleotides) that is not complementary to a sequence in the genomic DNA; and a primer that comprises a sequence of about 5 to about 25 contiguous nucleotides (e.g., between about 10 and 20 contiguous nucleotides, between about 5 and about 20 contiguous nucleotides, or between about 17 and about 25 contiguous nucleotides) of the tag sequence or complementary to the tag sequence.

In some examples, the kit can further include an enzyme-linked immunosorbent assay for detection of one or more saliva proteins (e.g., one of more of human statherin, human alpha-amylase, or human lysozyme), an antibody that binds specifically to a saliva protein (e.g., human statherin, human alpha-amylase, or human lysozyme) and/or a labeled substrate for detection of the activity (e.g., binding activity or enzymatic activity) of one or more saliva proteins (e.g., human statherin, human alpha-amylase, or human lysozyme).

In some examples of the kits, the tag sequence can include or can consist of SEQ ID NO: 1. As described above, a tag sequence can be selected or designed using methods well known in the art. In some examples of the kits, the at least two pairs of pre-amplification forward and reverse primers are designed to amplify genomic DNA that contains at least two (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs. For example, the at least two SNPs (e.g., at least three SNPs, at least six SNPs, at least eight SNPs, at least ten SNPs, or at least fourteen SNPs) in (i) have a minor allele frequency of >0.4. In any of the kits described herein, the at least two (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, fourteen, fifteen, sixteen, seventeen, eighteen, or nineteen) SNPs is selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some examples of the kits, the at least two SNPs (e.g., at least three, at least four, at least five, at least six, at least seven, at least eight, at least nine, at least ten, at least eleven, at least twelve, at least thirteen, at least fourteen, at least fifteen, at least sixteen, three, four, five, six, seven, eight, nine, ten, eleven, twelve, thirteen, or fourteen) SNPs are selected from the group of: rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some examples of the kits, the SNPs in (i) include rs279844, rs1058083, rs13182883, rs560681, rs740598, rs1358856, rs9951171, rs7520386, rs13218440, rs2272998, rs12997453, rs214955, rs13134862, rs1410059, rs33882, rs2503107, rs315791, rs6591147, and rs985492. In some examples of the kits, the SNPs in (i) include at least one (e.g., two, three, four, or five) SNP located on the Y chromosome.

In some examples, the kit contains: at least three pairs of pre-amplification forward and reverse primers that amplify at least three SNPs, at least four (e.g., four) pairs of pre-amplification forward and reverse primers that amplify at least four (e.g., four) SNPs, at least five (e.g., five) pairs of pre-amplification forward and reverse primers that amplify at least five (e.g., five) SNPs, at least six (e.g., six) pairs of pre-amplification forward and reverse primers that amplify at least six (e.g., six) SNPs, at least seven (e.g., seven) pairs of pre-amplification forward and reverse primers that amplify at least seven (e.g., seven) SNPs, at least eight (e.g., eight) pairs of pre-amplification forward and reverse primers that amplify at least eight (e.g., eight) SNPs, at least nine (e.g., nine) pairs of pre-amplification forward and reverse primers that amplify at least nine (e.g., nine) SNPs, at least ten (e.g., ten) pairs of pre-amplification forward and reverse primers that amplify at least ten (e.g., ten) SNPs, at least eleven (e.g., eleven) pairs of pre-amplification forward and reverse primers that amplify at least eleven (e.g., eleven) SNPs, at least twelve (e.g., twelve) pairs of pre-amplification forward and reverse primers that amplify at least twelve (e.g., twelve) SNPs, at least thirteen (e.g., thirteen) pairs of pre-amplification forward and reverse primers that amplify at least thirteen (e.g., thirteen) SNPs, or at least fourteen (e.g., fourteen) pairs of pre-amplification forward and reverse primers that amplify at least fourteen (e.g., fourteen) SNPs. In any of the kits, the at least two (e.g., at least three, at least four, at least five, at least six, at least seven, at least eight, at least nine, at least ten, at least eleven, at least twelve, at least thirteen, at least fourteen, three, four, five, six, seven, eight, nine, ten, eleven, twelve, thirteen, or fourteen) SNPs include at least one SNP from at least two different (e.g., three, four, five, six, seven, eight, nine, ten, eleven, or twelve different) chromosomes.

In some kits, the at least two (e.g., at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, three, four, five, six, seven, eight, nine, ten, eleven, twelve, thirteen, fourteen, fifteen, sixteen, seventeen, or eighteen) pairs of pre-amplification forward and reverse primers are selected from the group of: SEQ ID NO: 2 and SEQ ID NO: 3, respectively; SEQ ID NO: 4 and SEQ ID NO: 5, respectively; SEQ ID NO: 6 and SEQ ID NO: 7, respectively; SEQ ID NO: 8 and SEQ ID NO: 9, respectively; SEQ ID NO: 10 and SEQ ID NO: 11, respectively; SEQ ID NO: 12 and SEQ ID NO: 13, respectively; SEQ ID NO: 14 and SEQ ID NO: 15, respectively; SEQ ID NO: 16 and SEQ ID NO: 17, respectively; SEQ ID NO: 18 and SEQ ID NO: 19, respectively; SEQ ID NO: 20 and SEQ ID NO: 21, respectively; SEQ ID NO: 22 and SEQ ID NO: 23, respectively; SEQ ID NO: 24 and SEQ ID NO: 25, respectively; SEQ ID NO: 26 and SEQ ID NO: 27, respectively; SEQ ID NO: 28 and SEQ ID NO: 29, respectively; SEQ ID NO: 30 and SEQ ID NO: 31, respectively; SEQ ID NO: 32 and SEQ ID NO: 33, respectively; and SEQ ID NO: 34 and SEQ ID NO: 35, respectively.

Some examples of the kits further include a control DNA (e.g., a plant DNA or any of the exemplary control DNAs described herein). The control DNA can be, e.g., a gene or cDNA encoding spinach chloroplast ATP synthase gamma-subunit (AtpC). In some examples, the kit can further include a forward and reverse primer for amplifying the control DNA (e.g., a forward primer comprising SEQ ID NO: 36 and a reverse primer comprising SEQ ID NO: 37 for amplifying the cDNA or gene encoding AtpC).

The invention is further described in the following examples, which do not limit the scope of the invention described in the claims.

### EXAMPLES

### Example 1. Design and Testing of a Method for Detecting Synthetic Urine and Matching a Urine Sample to a Subject

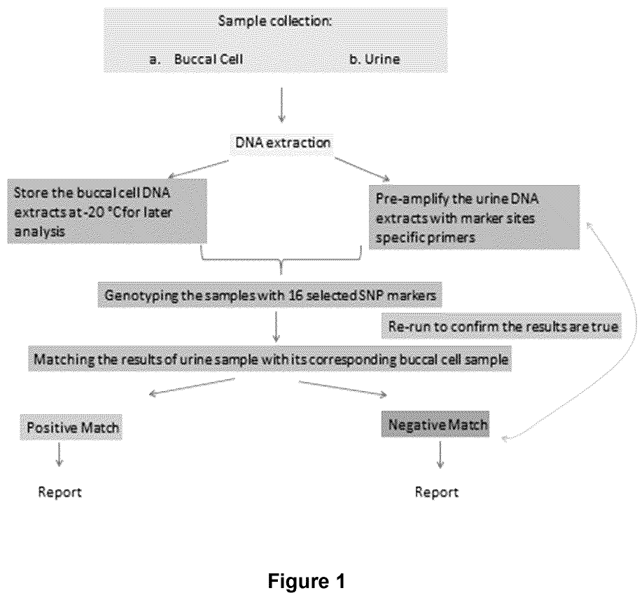

A method was designed to successfully verify the authenticity of a urine sample. A flow chart of the designed method (e.g., an exemplary method described herein) is shown in FIG. 1. In the flow chart shown in FIG. 1, a buccal cell sample and a urine sample are obtained from a subject, the genomic DNA isolated from each sample, and each isolated genomic DNA test sample was genotyped for 16 different SNPs shown in Table 1 below. Each SNP in Table 1 is highly polymorphic, each with a heterozygosity larger than 0.434. The genotyping of highly polymorphic SNPs allows for greater accuracy in matching the buccal cell sample to a urine sample.

TABLE 1
Set of SNP markers

| | | | | | | Avg. Het |
|---|---|---|---|---|---|---|
| | | Cytogenetic | Gene | | LifeTech | in 40 |
| Marker # | Chromosome | band position | Symble | SNP ID | Assay ID | population |
| 1 | 1 | p36 | PRDM2 | rs7520386 | C_{———}342791_10 | 0.477 |
| 2 | 1 | q21.3-22 | LY9 | rs560681 | C_{——}1006721_1_{—} | 0.434 |
| 3 | 18 | p11.3 | RAB31 | rs9951171 | C_{——}1371205_10 | 0.474 |
| 4 | 13 | q32.3 | PHGDHL1 | rs1058083 | C_{——}1619935_1_{—} | 0.464 |
| 5 | 6 | q22 | TRDN | rs1358856 | C_{——}2140539_10 | 0.473 |
| 6 | 6 | q25 | SYNE1 | rs214955 | C_{——}2515223_10 | 0.475 |
| 7 | 10 | q26 | HSPA12A | rs740598 | C_{——}3254784_10 | 0.463 |
| 8 | 4 | p12 | GABRA2 | rs279844 | C_{——}8263011_10 | 0.485 |
| 9 | 6 | p24-22.3 | HIVEP1 | rs13218440 | C_{——}9371416_10 | 0.457 |
| 10 | 6 | q24.3 | SASH1 | rs2272998 | C_{——}1256256_1_{—} | 0.468 |
| 11 | 2 | q31.3 | CERKL | rs12997453 | C_{——}1276208_10 | 0.445 |
| 12 | 4 | q21.1 | RCHY1 | rs13134862 | C_{——}1880371_10 | 0.456 |
| 13 | 5 | q31 | SPOCK | rs13182883 | C_{——}2556113_10 | 0.471 |
| 14 | 10 | q23.3-24.1 | SORBS1 | rs1410059 | C_{——}7538108_10 | 0.471 |
| 15 | Y | Chr.Y: 14847792 | USP9Y | rs2032597 | C_{——}1083231_10 | NA |
| 16 | Y | Chr.Y: 21867787 | KDM5D | rs2032631 | C_{——}2414552_30 | NA |

The 16 SNPs are located in 9 different chromosomes. There are four SNPs located on chromosome 6 at different band positions. These four SNPs are far apart from each other and cover about 40% of human chromosome 9. Because of the high polymorphism of the 16 SNPs, this set of SNPs results can be used to match a urine sample and buccal cell sample with an exclusion probability of over 99.9%. The first 9 somatic SNPs and the two Y-chromosome SNPs were tested and validated in the 384 well format on the real-time PCR system of Life Technologies.

### Development of Internal Positive Control

One of the means for adulterating a urine sample is to use synthetic urine. Detection of this type of sample (synthetic urine sample) is based on genotyping failure due to the lack of human genomic DNA in the sample. In order to confirm that failure of the genotyping is truly due to lack of human genomic DNA and not due to other factors, such as DNA extraction failure or the presence of an inhibitor of the genotyping reaction, an internal positive control was developed. The control DNA is present in the DNA extracts and its presence confirmed by a real-time PCR assay performed at the same time as the genotyping of the set of 16 SNPs. A positive amplification of the control DNA indicates that the DNA extraction process works and there is no reaction inhibitor. The criteria used to select the internal control were: non-human or bacterial DNA (e.g., so no cross-contamination is possible during sample handling and processing), the target gene is unique and will not have cross reaction with 16 SNPs or their amplification products, and there is any easy source for a large quantity of control DNA supply. Based on these criteria, the spinach chloroplast ATP synthase gamma-subunit (AtpC gene) was selected as the control DNA. The AtpC sequence information was obtained from the NCBI website (Genbank number X17257.1). A unique region of this gene which doesn't show homology to the human genome was selected via Basic Local Alignment Search Tool (BLAST) analysis using software available on the NCBI website. The real-time PCR primers and fluorescence dye labeled probe were designed, and the primers and probe were synthesized by Integrated DNA Technologies (Coralville, IA). The real-time PCR forward and reverse primers used to amplify the AtpC sequence are TCCCTCCTTATCCATCCTTACA (SEQ ID NO: 36) and CAGAGAGAAGGGT GTGATGTG (SEQ ID NO: 37). The probe used to detect the 108 base pair amplification AtpC product was labeled with FAM and had the sequence of TGGACAATTCCAACA CCCTCCTCC (SEQ ID NO: 41).

Spinach genomic DNA was extracted from spinach leaves purchased from a grocery store using the Qiagen DNeasy plant mini kit (Catalogue No. #69104) according to the manufacturer's protocol. The extracted genomic DNA was used as a template for the real-time assay with different primer and probe concentrations to establish the qPCR assay conditions for the target spinach AtpC gene. After establishing the assay conditions, different amounts of spinach DNA (ranging from 20 ng to 100 ng) were added to the cell pellet of a urine sample prior to DNA extraction. The extracted DNA was then used to genotype the 16 SNPs and also for detection of spinach AtpC gene using qPCR. The results were analyzed to determine if the spinach genomic DNA had interfered with the genotyping of the 16 SNPs and the minimum amount of spinach genomic DNA required for detection of the spinach AtpC gene in the isolated genomic DNA samples. The same experiment was also performed using a synthetic urine sample.

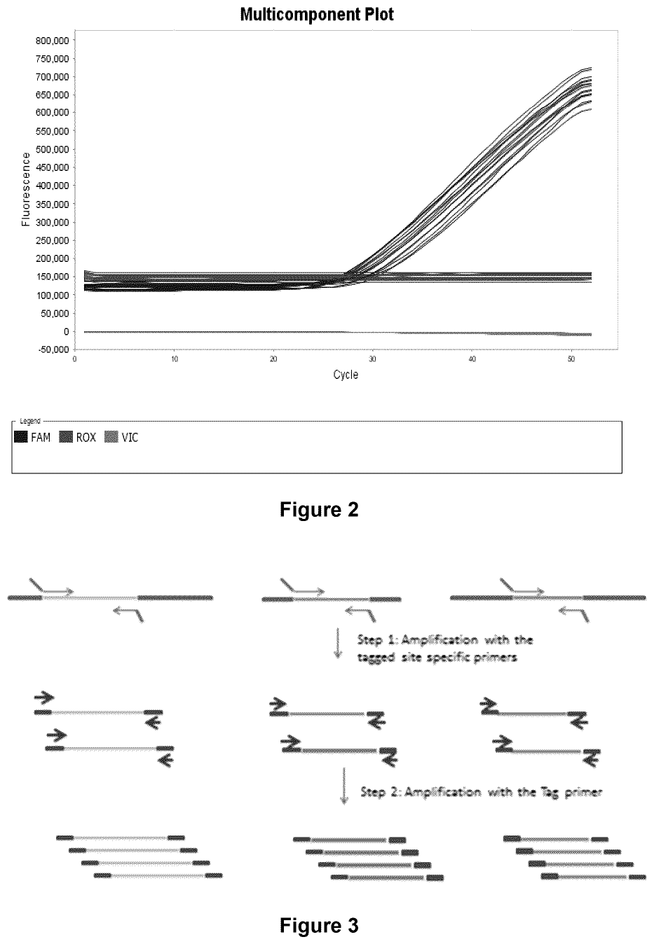

The results show that addition of 20 ng spinach genomic DNA to the cell pellet of the urine sample was sufficient as an internal control. However, for synthetic urine, a minimum of 50 ng of spinach DNA should be used for the internal control. These data show that in a situation where an isolated genomic DNA sample fails to give genotyping results, 50 ng or more spinach genomic DNA should be added to the cell pellet of the urine sample. The genomic DNA will then be isolated from the sample and the 16 SNPs genotyped and the spinach AtpC gene amplified and detected at the same time. When a sample is determined to be AtpC positive, but negative for more than one of the tested SNPs, this sample can be determined to be synthetic urine. The spinach AtpC gene amplification plot is represented in FIG. 2.

### Urine DNA Extraction Method

It is commonly accepted that DNA in urine is highly degraded and is not suitable for DNA genotyping. In order to isolate any small amounts of intact genomic DNA from urine, urine samples were high-speed centrifuged to collect a few cells in the urine, and the genomic DNA was then extracted from the isolated cells using a Qiagen buccal cell DNA extraction kit using the manufacturer's instructions, but with one added buffer #2 column-washing step added. In experiments to test the urine DNA extraction method, samples were collected from 6 female and 4 male individuals. Each individual provided a buccal cell sample and a urine sample in a single clinical visit. The DNA quantity and quality of the isolated DNA from each sample are summarized in Table 2.

As shown in Table 2, there is great variation in the isolated genomic DNA concentration from urine samples and buccal cell samples between individuals. Except patient VGTX0023, each individual's genomic DNA concentration in the buccal cell sample extracts is higher than that of the individual's urine sample. This difference is expected because there are more cells in the buccal cell samples than in the urine samples. The genomic DNA concentration from male urine samples is lower than that of female urine samples. A T-test comparing the DNA concentration between the male and female urine samples resulted in a p value of 0.04359.

An additional set of experiments was performed to determine the minimum volume of urine sample required for successful genotyping of the 16 SNPs. In these experiments, genomic DNA was extracted from the cell pellet from 1-mL, 2-mL, 4-mL, and 10-mL of two female and two male urine samples. The tests were performed in duplicates. The results are summarized in Table 3. For female urine samples, 1-mL urine is sufficient for successful SNP genotyping. For the males, at least 10-mL urine is required to generate reliable data.

TABLE 2
DNA concentration of tested samples

| | DNA | | | 5 fold | |
|---|---|---|---|---|---|
| Sample ID | (ng/μL) | 260/280 | 260/230 | dilution | Sex |
| U-VGTX0004 | 5.9 | 1.32 | 1.49 | 1.18 | F |
| U-VGTX0018 | 7.1 | 1.17 | 1.05 | 1.42 | F |
| U-VGTX0028 | 35 | 1.78 | 2.46 | 7 | F |
| U-VGTX0033 | 14.7 | 1.51 | 1.8 | 2.94 | F |
| U-VGTX0034 | 41 | 1.79 | 2.35 | 8.2 | F |
| U-VGTX0066 | 25.2 | 1.46 | 1.29 | 5.04 | F |
| Ave. | 21.48 | | | | |
| U-VGTX0023 | 15.1 | 1.23 | 0.97 | 3.02 | M |
| U-VGTX0024 | 4.1 | 1.2 | 7.48 | 0.82 | M |
| U-VGTX0053 | 2 | 1.36 | 1.21 | 0.4 | M |
| U-VGTX0065 | 3.6 | 1.34 | 3.51 | 0.72 | M |
| Ave. | 6.2 | | | | |
| B-VGTX0004 | 68.3 | 1.77 | 1.92 | 13.66 | F |
| B-VGTX0018 | 30.6 | 1.69 | 1.72 | 6.12 | F |
| B-VGTX0028 | 68.7 | 1.78 | 1.86 | 13.74 | F |
| B-VGTX0033 | 20.5 | 1.65 | 1.56 | 4.1 | F |
| B-VGTX0034 | 36.2 | 1.73 | 1.86 | 7.24 | F |
| B-VGTX0066 | 11.6 | 1.54 | 1.22 | 2.32 | F |
| Ave. | 39.32 | | | | |
| B-VGTX0023 | 11.1 | 1.43 | 1.08 | 2.22 | M |
| B-VGTX0024 | 21.6 | 1.67 | 1.63 | 4.32 | M |
| B-VGTX0053 | 18 | 1.61 | 1.67 | 3.6 | M |
| B-VGTX0065 | 34.5 | 1.8 | 1.96 | 6.9 | M |
| Ave. | 21.3 | | | | |

### SNP Genotyping

The genotyping of 11 of the 16 SNPs was performed using real-time PCR-based SNP genotyping assays. All assays are pre-made and QCed by Life Technologies. During the test validation process, the isolated genomic DNA samples from urine samples were discovered to contain reaction inhibitors. The isolated genomic DNA samples were diluted 5-fold with nuclease free water to insure the assay success in the 384 sample plate format. A template dilution test was performed and the results show that the SNP real-time genotyping assays in the 384 plate format provide for highly sensitive and reliable genotyping results for the 11 tested SNPs (the first 9 SNPs in Table 1 and the two Y chromosome SNPs) when a genomic DNA concentration between 0.625 ng/μL and 20 ng/μL was used.

TABLE 3
The 16-SNP Genotyping Test Results Using Different Volumes of Urine

| | | | | | | | | | |
|---|---|---|---|---|---|---|---|---|---|
| | Gene | NCBI | 10 | 1 | 1 | 2 | 2 | 4 | 4 |
| Assay ID | Symbol | SNP | mL | mL | mL | mL | mL | mL | mL |

Sample ID: VGTX0004 (Female)

| | | | | | | | | | |
|---|---|---|---|---|---|---|---|---|---|
| C_{———}342791_10 | PRDM2 | rs7520386 | A/G | A/G | A/G | A/G | A/G | A/G | A/G |
| C_{——}1006721_1_{—} | LY9 | rs560681 | A/A | A/A | A/A | A/A | A/A | A/A | A/A |
| C_{——}1371205_10 | RAB31 | rs9951171 | A/G | A/G | A/G | A/G | A/G | A/G | A/G |
| C_{——}1619935_1_{—} | UBAC2 | rs1058083 | G/G | G/G | G/G | G/G | G/G | G/G | G/G |
| C_{——}2140539_10 | TRDN | rs1358856 | A/C | A/C | A/C | A/C | A/C | A/C | A/C |
| C_{——}2556113_10 | SPOCK1 | rs13182883 | G/G | G/G | G/G | G/G | G/G | G/G | G/G |
| C_{——}3254784_10 | HSPA12A | rs740598 | A/A | A/A | A/A | A/A | A/A | A/A | A/A |
| C_{——}8263011_10 | GABRA2 | rs279844 | T/T | T/T | T/T | T/T | T/T | T/T | T/T |
| C_{——}9371416_10 | HIVEP1 | rs13218440 | A/G | A/G | A/G | A/G | A/G | A/G | A/G |

Sample ID: VGTX0007 (Female)

| | | | | | | | | | |
|---|---|---|---|---|---|---|---|---|---|
| C_{———}342791_10 | PRDM2 | rs7520386 | A/G | A/G | A/G | A/G | A/G | A/G | A/G |
| C_{——}1006721_1_{—} | LY9 | rs560681 | A/G | A/G | A/G | A/G | A/G | A/G | A/G |
| C_{——}1371205_10 | RAB31 | rs9951171 | A/G | A/G | A/G | A/G | A/G | A/G | A/G |
| C_{——}1619935_1_{—} | UBAC2 | rs1058083 | G/G | G/G | G/G | G/G | G/G | G/G | G/G |
| C_{——}2140539_10 | TRDN | rs1358856 | A/C | A/C | A/C | A/C | A/C | A/C | A/C |
| C_{——}2556113_10 | SPOCK1 | rs13182883 | A/G | A/G | A/G | A/G | A/G | A/G | A/G |
| C_{——}3254784_10 | HSPA12A | rs740598 | A/G | A/G | A/G | A/G | A/G | A/G | A/G |
| C_{——}8263011_10 | GABRA2 | rs279844 | A/T | A/T | A/T | A/T | A/T | A/T | A/T |
| C_{——}9371416_10 | HIVEP1 | rs13218440 | A/G | A/G | A/G | A/G | A/G | A/G | A/G |

Sample ID: VGTX0023 (male)

| | | | | | | | | | |
|---|---|---|---|---|---|---|---|---|---|
| C_{———}342791_10 | PRDM2 | rs7520386 | A/G | NoCall | A/A | NoCall | NoCall | A/G | NoCall |
| C_{——}1006721_1_{—} | LY9 | rs560681 | A/A | NoCall | NoCall | NoCall | NoCall | A/A | NoCall |
| C_{——}1371205_10 | RAB31 | rs9951171 | G/G | NoCall | NoCall | NoCall | G/G | G/G | G/G |
| C_{——}1619935_1_{—} | UBAC2 | rs1058083 | G/G | NoCall | A/G | NoCall | NoCall | NoCall | NoCall |
| C_{——}2140539_10 | TRDN | rs1358856 | C/C | NoCall | NoCall | NoCall | NoCall | c/C | NoCall |
| C_{——}2556113_10 | SPOCK1 | rs13182883 | G/G | NoCall | NoCall | NoCall | UND | G/G | G/G |
| C_{——}3254784_10 | HSPA12A | rs740598 | A/G | NoCall | NoCall | NoCall | NoCall | NoCall | NoCall |
| C_{——}8263011_10 | GABRA2 | rs279844 | A/T | NoCall | NoCall | NoCall | NoCall | NoCall | T/T |
| C_{——}9371416_10 | HIVEP1 | rs13218440 | A/G | NoCall | NoCall | NoCall | NoCall | A/A | A/A |

Sample ID: VGTX0038 (male)

| | | | | | | | | | |
|---|---|---|---|---|---|---|---|---|---|
| C_{———}342791_10 | PRDM2 | rs7520386 | A/G | NoCall | A/G | A/A | NoCall | NoCall | NoCall |
| C_{——}1006721_1_{—} | LY9 | rs560681 | A/G | NoCall | A/A | NoCall | NoCall | G/G | NoCall |
| C_{——}1371205_10 | RAB31 | rs9951171 | G/G | NoCall | NoCall | NoCall | NoCall | NoCall | G/G |
| C_{——}1619935_1_{—} | UBAC2 | rs1058083 | G/G | NoCall | NoCall | G/G | NoCall | G/G | G/G |
| C_{——}2140539_10 | TRDN | rs1358856 | C/C | NoCall | NoCall | C/C | NoCall | C/C | NoCall |
| C_{——}2556113_10 | SPOCK1 | rs13182883 | G/G | NoCall | G/G | G/G | G/G | G/G | G/G |
| C_{——}3254784_10 | HSPA12A | rs740598 | A/A | NoCall | NoCall | NoCall | NoCall | A/A | A/A |

NoCall: No genotype calls were made.

Several measures were implemented in the system to prevent sample cross-contamination and ensure test accuracy. These measures are listed below.
1) The DNA extraction, real-time PCR reaction setting up and real-time PCR were carried out in three separate rooms with an isolated air circulation system to prevent airborne contamination.
2) Only filtered tips were used throughout the testing process from sample preparation to setting up the real-time PCR test.
3) The PCR master mix was made in a PCR hood.
4) The working space was cleaned by 10% bleach, followed by water, and 70% ethanol prior to and after sample processing.
5) Each sample was tested in duplicate. If there was a genotyping discrepancy between two replicates, the sample was re-tested to ensure the accuracy of the result.
6) Each urine sample was processed in two aliquots. One aliquot was used for genotype testing and the other one is stored as a backup. In an instance where the urine sample has a detected mismatch between the buccal cell marker for one or two SNPs, the backup aliquot of the urine sample was re-extracted and tested to confirm whether the mismatch is true and not the result of cross-contamination.

### Test Accuracy and Reproducibility Validation

The accuracy of the method shown in FIG. 1 was evaluated in two parts.

First, Sanger sequencing was used to confirm that all of the real-time PCR genotyping results were correct across DNA samples of 15 unrelated individuals. In these experiments, the primers flanking each of the SNPs were designed using the “Primer-Blast” program on the NCBI website and synthesized by Integrated DNA Technologies. The sites of the 15 individual samples were then PCR-amplified and sequenced using a Sanger sequencer. The sequence results of the SNPs were then compared with the real-time PCR results (Table 4). All the sequence results matched between the two methods (Sanger sequencing and the real-time PCR results).

Second, the urine genomic DNA samples of the 10 individuals whose DNA genotype results of the SNPs have been confirmed by Sanger sequencing were genotyped to see if the urine samples can be matched correctly to the buccal cell samples. These data are shown in Table 5.

TABLE 4
Results of the Real-Time PCR Assay and
Sanger Sequencing Correlation Study

| Sample ID | Assay ID | QS | Seq | Matched |
|---|---|---|---|---|
| NA01251 | C____342791_10 | A/G | A/G | yes |
| | C___1006721_1_ | A/G | A/G | yes |
| | C___1371205_10 | A/G | A/G | yes |
| | C___1619935_1_ | A/G | A/G | yes |
| | C___2140539_10 | A/C | C/A | yes |
| | C___2556113_10 | A/G | A/G | yes |
| | C___3254784_10 | A/G | A/G | yes |
| | C___8263011_10 | T/T | T/T | yes |
| | C___9371416_10 | A/A | A/A | yes |
| NA02016 | C____342791_10 | A/A | A/A | yes |
| | C___1006721_1_ | A/G | A/G | yes |
| | C___1371205_10 | A/G | A/G | yes |
| | C___1619935_1_ | G/G | G/G | yes |
| | C___2140539_10 | A/C | C/A | yes |
| | C___2556113_10 | A/G | A/G | yes |
| | C___3254784_10 | A/G | A/G | yes |
| | C___8263011_10 | A/A | A/A | yes |
| | C___9371416_10 | A/G | G/A | yes |
| NA10839 | C____342791_10 | A/A | A/A | yes |
| | C___1006721_1_ | A/A | A/A | yes |
| | C___1371205_10 | G/G | G/G | yes |
| | C___1619935_1_ | G/G | G/G | yes |
| | C___2140539_10 | A/C | C/A | yes |
| | C___2556113_10 | A/A | A/A | yes |
| | C___3254784_10 | A/G | A/G | yes |
| | C___8263011_10 | A/T | A/T | yes |
| | C___9371416_10 | A/G | G/A | yes |
| NA17138 | C____342791_10 | G/G | G/G | yes |
| | C___1006721_1_ | A/G | A/G | yes |
| | C___1371205_10 | A/G | A/G | yes |
| | C___1619935_1_ | A/G | A/A | yes |
| | C___2140539_10 | C/C | C/C | yes |
| | C___2556113_10 | A/G | A/G | yes |
| | C___3254784_10 | G/G | G/G | yes |
| | C___8263011_10 | A/A | A/A | yes |
| | C___9371416_10 | A/G | G/A | yes |
| NA17221 | C____342791_10 | G/G | G/G | yes |
| | C___1006721_1_ | A/G | A/G | yes |
| | C___1371205_10 | A/G | A/G | yes |
| | C___1619935_1_ | G/G | G/G | yes |
| | C___2140539_10 | A/C | C/A | yes |
| | C___2556113_10 | A/G | A/G | yes |
| | C___3254784_10 | A/G | A/G | yes |
| | C___8263011_10 | T/T | T/T | yes |
| | C___9371416_10 | G/G | G/G | yes |
| VGTX0004 | C____342791_10 | A/G | A/G | yes |
| | C___1006721_1_ | A/A | A/A | yes |
| | C___1371205_10 | A/G | A/G | yes |
| | C___1619935_1_ | G/G | G/G | yes |
| | C___2140539_10 | A/C | C/A | yes |
| | C___2556113_10 | G/G | G/G | yes |
| | C___3254784_10 | A/A | A/A | yes |
| | C___8263011_10 | T/T | T/T | yes |
| | C___9371416_10 | A/G | G/A | yes |
| VGTX0018 | C____342791_10 | A/G | A/G | yes |
| | C___1006721_1_ | A/A | A/A | yes |
| | C___1371205_10 | A/G | A/G | yes |
| | C___1619935_1_ | A/A | A/A | yes |
| | C___2140539_10 | A/A | A/A | yes |
| | C___2556113_10 | A/A | A/A | yes |
| | C___3254784_10 | G/G | G/G | yes |
| | C___8263011_10 | T/T | T/T | yes |
| | C___9371416_10 | A/G | G/A | yes |
| VGTX0023 | C____342791_10 | A/G | A/G | yes |
| | C___1006721_1_ | A/A | A/A | yes |
| | C___1371205_10 | G/G | G/G | yes |
| | C___1619935_1_ | G/G | G/G | yes |
| | C___2140539_10 | C/C | C/C | yes |
| | C___2556113_10 | G/G | G/G | yes |
| | C___3254784_10 | A/G | A/G | yes |
| | C___8263011_10 | A/T | A/T | yes |
| | C___9371416_10 | A/G | G/A | yes |
| VGTX0024 | C____342791_10 | A/A | A/A | yes |
| | C___1006721_1_ | G/G | G/G | yes |
| | C___1371205_10 | A/G | A/G | yes |
| | C___1619935_1_ | A/G | A/G | yes |
| | C___2140539_10 | A/C | C/A | yes |
| | C___2556113_10 | G/G | G/G | yes |
| | C___3254784_10 | A/G | A/G | yes |
| | C___8263011_10 | A/T | A/T | yes |
| | C___9371416_10 | A/G | G/A | yes |
| VGTX0028 | C____342791_10 | G/G | G/G | yes |
| | C___1006721_1_ | G/G | G/G | yes |
| | C___1371205_10 | A/A | A/A | yes |
| | C___1619935_1_ | A/G | A/G | yes |
| | C___2140539_10 | A/A | A/A | yes |
| | C___2556113_10 | G/G | G/G | yes |
| | C___3254784_10 | A/A | A/A | yes |
| | C___8263011_10 | A/T | A/T | yes |
| | C___9371416_10 | A/G | G/A | yes |
| VGTX0033 | C____342791_10 | A/G | A/G | yes |
| | C___1006721_1_ | A/G | A/G | yes |
| | C___1371205_10 | G/G | G/G | yes |
| | C___1619935_1_ | A/A | A/A | yes |
| | C___2140539_10 | A/C | C/A | yes |
| | C___2556113_10 | A/G | A/G | yes |
| | C___3254784_10 | A/A | A/A | yes |
| | C___8263011_10 | A/T | A/T | yes |
| | C___9371416_10 | G/G | G/G | yes |
| VGTX0034 | C____342791_10 | A/A | A/A | yes |
| | C___1006721_1_ | A/G | A/G | yes |
| | C___1371205_10 | A/A | A/A | yes |
| | C___1619935_1_ | A/A | A/A | yes |
| | C___2140539_10 | A/A | A/A | yes |
| | C___2556113_10 | A/G | A/G | yes |
| | C___3254784_10 | G/G | G/G | yes |
| | C___8263011_10 | A/A | A/A | yes |
| | C___9371416_10 | G/G | G/G | yes |
| VGTX0053 | C____342791_10 | A/G | A/G | yes |
| | C___1006721_1_ | A/G | A/G | yes |
| | C___1371205_10 | A/A | A/A | yes |
| | C___1619935_1_ | A/G | A/G | ye |
| | C___2140539_10 | A/C | C/A | yes |
| | C___2556113_10 | G/G | G/G | yes |
| | C___3254784_10 | A/A | A/A | yes |
| | C___8263011_10 | T/T | T/T | yes |
| | C___9371416_10 | A/G | G/A | yes |
| VGTX0065 | C____342791_10 | A/A | A/A | yes |
| | C___1006721_1_ | A/G | A/G | yes |
| | C___1371205_10 | A/G | A/G | yes |
| | C___1619935_1_ | A/G | A/G | yes |
| | C___2140539_10 | A/A | A/A | yes |
| | C___2556113_10 | A/G | A/G | yes |
| | C___3254784_10 | A/A | A/A | yes |
| | C___8263011_10 | A/A | A/A | yes |
| | C___9371416_10 | A/G | G/A | yes |
| VGTX0066 | C____342791_10 | A/G | A/G | yes |
| | C___1006721_1_ | A/A | A/A | yes |
| | C___1371205_10 | G/G | G/G | yes |
| | C___1619935_1_ | A/G | A/G | yes |
| | C___2140539_10 | A/C | C/A | yes |
| | C___2556113_10 | G/G | G/G | yes |
| | C___3254784_10 | A/G | A/G | yes |
| | C___8263011_10 | A/T | A/T | yes |
| | C___9371416_10 | A/G | G/A | yes |

GS: QuantStudio- real-time PCR method; Seq: Sanger sequencing.

All the genotypes matched between the buccal cell samples and the urine samples. Thus, the methods described in this Example have 100% accuracy in matching the genotype of 9 of the SNPs (the eleven SNPs minus the two Y chromosome SNPs) in the buccal cell samples to the urine cell samples. The same validation test was performed for the two Y chromosome SNPs and the test accuracy was also 100%.

### Test Reproducibility Evaluated by Testing 10 Paired Urine and Buccal Cell DNA Samples

A further set of experiments was performed to test the reproducibility of the matching of urine and buccal cell samples from the same subject. In these experiments, 10 paired urine and buccal cell samples were tested in duplicate in 4 separate runs on three days. The results of these experiments are shown in Table 6. The data shows that 20 samples, 10 buccal cell samples and 10 urine sample DNA extracts, were tested across the 9 assays (9 SNPs total) in 4 separate runs. The total number of tests done for each assay was 140 for 7 of the 9 markers. Due to a noticed operator error in which genomic DNA was not added to the reaction wells for assay C_1006721_1 and C_2140539_10 was 127. All tests gave correct genotype matching between the urine and buccal cell samples (100% accuracy). Out of the 9 assays, 5 resulted in correct matching between the urine and buccal cell samples for all replicates (100% reproducibility). The other two assays had one reaction fail resulting in 99% reproducibility. One assay had 6 failed reactions and the other had 5 failed reactions resulting in 95% and 96% reproducibility for these two assays.

### Test Sensitivity and Specificity

A double blind test was performed to further evaluate the test sensitivity and specificity. For this test, a total of 47 individuals donated their buccal cell and urine samples. The individuals who obtained these samples were not involved in the centrifugation of the samples, the extraction of the genomic DNA from the samples, the determination of the genotype of the SNPs, or the detection of the control DNA in the samples. The individuals who obtained the samples put the samples into matched pairs and mismatched pairs of samples, and also substituted a few urine samples with synthetic urine. The resulting pairs of samples were processed and tested (Table 7). Out of the 47 samples, 3 urine samples failed to produce reliable genotyping results due to lack or poor genomic DNA quality (marked dark gray in Table 7) and where omitted from the data analysis. For the remaining 44 paired samples, the assay correctly identified the 10 negative-matched pairs, with three of the urine samples being identified as being synthetic urine. None of the positive-matched pairs were identified wrongly as mismatched. Thus, the test was demonstrated in this experiment to have 100% sensitivity and 100% specificity (Table 7). Two urine samples were labeled #19 by mistake (marked with asterisks). The test result identified the correct #19 urine sample which matched with its buccal cell sample. The second #19 urine sample was intended to be paired with the #36 buccal cell sample, because the #36 urine sample was missing. The test

TABLE 5
Genotyping Results of the Buccal Cell DNA Extracts
versus the Urine Sample DNA Extracts

| | NCBI SNP | Sample ID | | Sample ID | | |
|---|---|---|---|---|---|---|
| Assay ID | Reference | (Buccal) | Call | (Urine) | Call | Matched |
| C___2556113_10 | rs13182883 | B-VGTX0004 | G/G | U-VGTX0004 | G/G | Yes |
| C___1006721_1_ | rs560681 | B-VGTX0004 | A/A | U-VGTX0004 | A/A | Yes |
| C___3254784_10 | rs740598 | B-VGTX0004 | A/A | U-VGTX0004 | A/A | Yes |
| C___2140539_10 | rs1358856 | B-VGTX0004 | A/C | U-VGTX0004 | A/C | Yes |
| C___1371205_10 | rs9951171 | B-VGTX0004 | A/G | U-VGTX0004 | A/C | Yes |
| C___1619935_1_ | rs1058083 | B-VGTX0033 | A/A | U-VGTX0033 | A/A | Yes |
| C___2556113_10 | rs13182883 | B-VGTX0034 | A/G | U-VGTX0034 | A/G | Yes |
| C___1006721_1_ | rs560681 | B-VGTX0034 | A/G | U-VGTX0034 | A/G | Yes |
| C___3254784_10 | rs740598 | B-VGTX0034 | G/G | U-VGTX0034 | G/G | Yes |
| C___2140539_10 | rs1358856 | B-VGTX0034 | A/A | U-VGTX0034 | A/A | Yes |
| C___1371205_10 | rs9951171 | B-VGTX0034 | A/A | U-VGTX0034 | A/A | Yes |
| C____342791_10 | rs7520386 | B-VGTX0034 | A/A | U-VGTX0034 | A/A | Yes |
| C___9371416_10 | rs13218440 | B-VGTX0034 | G/G | U-VGTX0034 | G/G | Yes |
| C___8263011_10 | rs279844 | B-VGTX0034 | A/A | U-VGTX0034 | A/A | Yes |
| C___1619935_1_ | rs1058083 | B-VGTX0034 | A/A | U-VGTX0034 | A/A | Yes |
| C___2556113_10 | rs13182883 | B-VGTX0053 | G/G | U-VGTX0053 | G/G | Yes |
| C___1006721_1_ | rs560681 | B-VGTX0053 | A/G | U-VGTX0053 | A/G | Yes |
| C___3254784_10 | rs740598 | B-VGTX0053 | A/A | U-VGTX0053 | A/A | Yes |
| C___2140539_10 | rs1358856 | B-VGTX0053 | A/C | U-VGTX0053 | A/C | Yes |
| C___1371205_10 | rs9951171 | B-VGTX0053 | A/A | U-VGTX0053 | A/A | Yes |
| C____342791_10 | rs7520386 | B-VGTX0053 | A/G | U-VGTX0053 | A/G | Yes |
| C___9371416_10 | rs13218440 | B-VGTX0053 | A/G | U-VGTX0053 | A/G | Yes |
| C___8263011_10 | rs279844 | B-VGTX0053 | T/T | U-VGTX0053 | T/T | Yes |
| C___1619935_1_ | rs1058083 | B-VGTX0053 | A/G | U-VGTX0053 | A/G | Yes |
| C___2556113_10 | rs13182883 | B-VGTX0065 | A/G | U-VGTX0065 | A/G | Yes |
| C___1006721_1_ | rs560681 | B-VGTX0065 | A/G | U-VGTX0065 | A/G | Yes |
| C___3254784_10 | rs740598 | B-VGTX0065 | A/A | U-VGTX0065 | A/A | Yes |
| C___2140539_10 | rs1358856 | B-VGTX0065 | A/A | U-VGTX0065 | A/A | Yes |
| C___1371205_10 | rs9951171 | B-VGTX0065 | A/G | U-VGTX0065 | A/G | Yes |
| C____342791_10 | rs7520386 | B-VGTX0065 | A/A | U-VGTX0065 | A/A | Yes |
| C___9371416_10 | rs13218440 | B-VGTX0065 | A/G | U-VGTX0065 | A/G | Yes |
| C___8263011_10 | rs279844 | B-VGTX0065 | A/A | U-VGTX0065 | A/A | Yes |
| C___1619935_1_ | rs1058083 | B-VGTX0065 | A/G | U-VGTX0065 | A/G | Yes |
| C___2556113_10 | rs13182883 | B-VGTX0066 | G/G | U-VGTX0066 | G/G | Yes |
| C___1006721_1_ | rs560681 | B-VGTX0066 | A/A | U-VGTX0066 | A/A | Yes |
| C___3254784_10 | rs740598 | B-VGTX0066 | A/G | U-VGTX0066 | A/G | Yes |
| C___2140539_10 | rs1358856 | B-VGTX0066 | A/C | U-VGTX0066 | A/C | Yes |
| C___1371205_10 | rs9951171 | B-VGTX0066 | G/G | U-VGTX0066 | G/G | Yes |
| C____342791_10 | rs7520386 | B-VGTX0066 | A/G | U-VGTX0066 | A/G | Yes |
| C___9371416_10 | rs13218440 | B-VGTX0066 | A/G | U-VGTX0066 | A/G | Yes |
| C___8263011_10 | rs279844 | B-VGTX0066 | A/T | U-VGTX0066 | A/T | Yes |
| C___1619935_1_ | rs1058083 | B-VGTX0066 | A/G | U-VGTX0066 | A/G | Yes |
| C___2556113_10 | rs13182883 | B_VGTX0028 | G/G | U_VGTX0028 | G/G | Yes |
| C___1006721_1_ | rs560681 | B_VGTX0028 | G/G | U_VGTX0028 | G/G | Yes |
| C___3254784_10 | rs740598 | B_VGTX0028 | A/A | U_VGTX0028 | A/A | Yes |
| C___2140539_10 | rs1358856 | B_VGTX0028 | A/A | U_VGTX0028 | A/A | Yes |
| C___1371205_10 | rs9951171 | B_VGTX0028 | A/A | U_VGTX0028 | A/A | Yes |
| C____342791_10 | rs7520386 | B_VGTX0028 | G/G | U_VGTX0028 | G/G | Yes |
| C___9371416_10 | rs13218440 | B_VGTX0028 | A/G | U_VGTX0028 | A/G | Yes |
| C___8263011_10 | rs279844 | B_VGTX0028 | A/T | U_VGTX0028 | A/T | Yes |
| C___1619935_1_ | rs1058083 | B_VGTX0028 | A/G | U_VGTX0028 | A/G | Yes |

B_: Buccal cell sample; U_: Urine Cell Sample.

TABLE 6
Test Reproducibility of the 9 Somatic SNPs

| | No. of | No. of | No. of | No. of | No. of | Call | | |
|---|---|---|---|---|---|---|---|---|
| Assay ID | Samples | Runs | Tests | NoCalls | Miss Calls | Rate | Reproducibility | Accuracy |
| C____342791_10 | 20 | 4 | 140 | 0 | 0 | 100.0% | 100.0% | 100.0% |
| C___1006721_1_ | 20 | 4 | 130 | 0 | 0 | 100.0% | 100.0% | 100.0% |
| C___1371205_10 | 20 | 4 | 140 | 0 | 0 | 100.0% | 100.0% | 100.0% |
| C___1619935_1_ | 20 | 4 | 140 | 0 | 0 | 100.0% | 100.0% | 100.0% |
| C___2140539_10 | 20 | 4 | 127 | 6 | 0 | 95.3% | 95.3% | 100.0% |
| C___2556113_10 | 20 | 4 | 140 | 1 | 0 | 99.3% | 99.3% | 100.0% |
| C___3254784_10 | 20 | 4 | 140 | 1 | 0 | 99.3% | 99.3% | 100.0% |
| C___8263011_10 | 20 | 4 | 140 | 5 | 0 | 96.4% | 96.4% | 100.0% |
| C___9371416_10 | 20 | 4 | 140 | 0 | 0 | 100.0% | 100.0% | 100.0% |

showed that the second #19 urine sample did not match with the #36 buccal cell sample, but matched with the #3 buccal cell sample. These results demonstrate that the test can be used to detect sample adultery via substitution with another person's urine and can also be used as a quality control tool in the lab to eliminate operator error in sample handling.

### Scale-Up of Test Capacity

The test capacity in these examples, using the 384 well format, is 45 samples per day. One way to scale-up these methods is perform the genotyping using a TaqMan® OpenArray (Life Technologies). Using an OpenArray®, 16 SNP assays can be tested for each sample and each array can run 68 samples at once. The full capacity using an OpenArray® will be 272 samples per day.

An important element for the success of the TaqManR OpenArray genotyping assay using isolated DNA from urine samples is to have good quantity and quality of DNA for the test. Isolated DNA from 32 urine samples were tested on a pre-made genotyping TaqManR OpenArray® with 32 assays from Life Technologies (Catalog #4475386). More than 88% of the samples failed to produce a genotype for all assays. This result suggests that isolated DNA from the urine sample is not optimal for use in a Taqman® OpenArray® assay, and suggests that a pre-amplification step may be necessary to ensure success.

TABLE 7
Double-Blind Test Results

| Urine ID# for | Buccal Cell ID# | True | |
|---|---|---|---|
| Genetic Group | for Genetic Group | Status | Detected |
| 1 | 1 | Positive | Positive |
| | | Match | Match |
| 2 | 2 | Positive | Positive |
| | | Match | Match |
| 3 | 3 | Synthetic | Synthetic |
| 4 | 4 | Positive | Positive |
| | | Match | Match |
| 5 | 5 | Positive | Positive |
| | | Match | Match |
| 6 | 6 | Positive | Positive |
| | | Match | Match |
| 7 | 7 | Positive | Positive |
| | | Match | Match |
| 8 | 8 | Positive | Positive |
| | | Match | Match |
| 9 | 9 | Synthetic | Synthetic |
| 10 | 10 | Positive | Positive |
| | | Match | Match |
| 11 | 11 | Positive | NA |
| | | Match | |
| 12 | 12 | Positive | Positive |
| | | Match | Match |
| 13 | 13 | Positive | Positive |
| | | Match | Match |
| 14 | 14 | Positive | Positive |
| | | Match | Match |
| 15 | 15 | Synthetic | Synthetic |
| 16 | 16 | Positive | Positive |
| | | Match | Match |
| 17 | 17 | Positive | Positive |
| | | Match | Match |
| 18 | 18 | Positive | Positive |
| | | Match | Match |
| 19-1* | 19 | Positive | Positive |
| | | Match | Match |
| 20 | 20 | Negative | NA |
| | | Match | |
| 21 | 21 | Positive | Positive |
| | | Match | Match |
| 22 | 22 | Positive | Positive |
| | | Match | Match |
| 23 | 23 | Positive | Positive |
| | | Match | Match |
| 24 | 24 | Positive | Positive |
| | | Match | Match |
| 25 | 25 | Positive | Positive |
| | | Match | Match |
| 26 | 26 | Negative | Negative |
| | | Match | Match |
| 27 | 27 | Positive | Positive |
| | | Match | Match |
| 28 | 28 | Positive | Positive |
| | | Match | Match |
| 29 | 29 | Positive | Positive |
| | | Match | Match |
| 30 | 30 | Positive | Positive |
| | | Match | Match |
| 31 | 31 | Positive | Positive |
| | | Match | Match |
| 32 | 32 | Positive | Positive |
| | | Match | Match |
| 33 | 33 | Positive | Positive |
| | | Match | Match |
| 34 | 34 | Positive | Positive |
| | | Match | Match |
| 35 | 35 | Negative | NA |
| | | Match | |
| 19-2* | 36 | Negative | Negative |
| | | Match | Match |
| 37 | 37 | Positive | Positive |
| | | Match | Match |
| 38 | 38 | Positive | Positive |
| | | Match | Match |
| 39 | 39 | Positive | Positive |
| | | Match | Match |
| 40 | 40 | Positive | Positive |
| | | Match | Match |
| 41 | 41 | Negative | Negative |
| | | Match | Match |
| 42 | 42 | Negative | Negative |
| | | Match | Match |
| 43 | 43 | Negative | Negative |
| | | Match | Match |
| 44 | 44 | Positive | Positive |
| | | Match | Match |
| 45 | 45 | Negative | Negative |
| | | Match | Match |
| 46 | 46 | Negative | Negative |
| | | Match | Match |
| 47 | 47 | Positive | Positive |
| | | Match | Match |

Positive Match: Genotypes of the urine sample matched with its corresponding buccal cell sample.
Negative Match: Genotypes of the urine sample did not match with its corresponding buccal cell sample.
Synthetic: Synthetic urine. It is also counted as a negative match.
*Two urine samples were mislabeled as #19 by mistake. Test result matched one of the two #19 urine samples with the corresponding #19 buccal cell sample. The second of the two #19 urine samples did not match with its intended assignee #36 buccal cell sample, but did match with the #3 buccal cell sample.

### Pre-Amplification Experimental Design

SNP site amplification was performed to ensure sufficient template DNA for the genotyping assays performed using high throughput TaqMan OpenArray® technology. The goal of the pre-amplification was to uniformly amplify the 16 SNP sites at the same time in a single reaction. In the traditional site specific amplification method, the specific target is amplified by site specific primers. There are often a limited number of targets that one can amplify in a reaction due to interference caused by non-specific priming and primer to primer interaction interference. In these experiments, a two-step PCR approach was developed using tagged primers to overcome the challenge of multi-plexing PCR amplification. The experimental design is illustrated in FIG. 3. All of the target site-specific primers were designed to have a universal tag at their ends. In the first step, the tagged site-specific primers identify the sites and amplify the specific regions. In the second step, the amplification is primarily carried out via the universal tag using the products from the first step as the templates. All of the site specific primers were designed to generate fragments with a size ranging from 250 base pairs to 300 base pairs for all targets. Because the amplified targets from step 1 are uniform in their sizes and have the universal tag on both ends, the second step amplification with the universal tag primer can amplify all targets uniformly without the problems of target specific bias or multiplex interference which are commonly present in the standard PCR multiplex reactions.

### Primer Design

The universal tag used in the pre-amplification primers was generated by randomly combining the G, A, T, and C nucleotides to make 20 nucleotide-long oligonucleotide sequences. These random sequences were then BLASTed against the human genome database to identify a unique sequence that did not show homology to any existing sequence in the human genome database.

The site specific primers designed for the 16 different SNPs shown in Table 1 were designed using the Primer-Blast program at the NBCI site to generate fragments with a size ranging from 250 base pairs to 300 base pairs. The primers were then tagged with the universal tag. The resulting primer sequences were analyzed using the Oligo analyzer at the Integrated DNA Technologies website to ensure the functionality of the primers. Any primers with a stable internal secondary structure (dG value less than −3) were redesigned. The resulting primer sequences (SEQ ID NOs: 2-33) are summarized in Table 8, shown in the order of the corresponding SNPs in Table 1 that they amplify. The universal tag sequence in each primer in Table 8 is CAAGATGCTACGCTTC AGTC (SEQ ID NO: 1).

TABLE 8
The Pre-Amplification Primers of the 16 SNPs (SEQ ID NOs: 2-33 and 1)

| Forward Primer | Primer sequence | Reverse Primer | Primer sequence |
|---|---|---|---|
| Tg-rs13182883F | CAAGATGCTACGCTTCAGTCAGGAGACTATGAGG | Tg-rs13182883R | CAAGATGCTACGCTTCAGTCC |
| | TGTGTCTCT | | CTGTGCACCTCGATTGAA |
| Tg-rs560681F | CAAGATGCTACGCTTCAGTCCCAAGGGGAATCAC | Tg-rs560681R | CAAGATGCTACGCTTCAGTCT |
| | ACCTC | | CTGTGGAAGCATGCCACTC |
| Tg-rs740598F | CAAGATGCTACGCTTCAGTCTGCTGAGCCACTCT | Tg-rs740598R | CAAGATGCTACGCTTCAGTCT |
| | TTCAGG | | TCCGGGATGTCCCGTCTTA |
| Tg-rs1358856F | CAAGATGCTACGCTTCAGTCACAGGCAAAGAGGA | Tg-rs1358856R | CAAGATGCTACGCTTCAGTCT |
| | ACATACAGT | | GCTGGCAGTGTTATTTCTTTC |
| | | | TC |
| Tg-rs9951171F | CAAGATGCTACGCTTCAGTCCTCGTTGTTCCTCT | Tg-rs9951171R | CAAGATGCTACGCTTCAGTCC |
| | GGG | | TGTTCAAGGGAAGCCTGT |
| Tg-rs7520386F | CAAGATGCTACGCTTCAGTCGGATCAGGAAACAG | Tg-rs7520386R | CAAGATGCTACGCTTCAGTCG |
| | GGAGCC | | AAGACTCTGTCCCAGCCAC |
| Tg-rs13218440F | CAAGATGCTACGCTTCAGTCGCTTCTTCTGCCAC | Tg-rs13218440R | CAAGATGCTACGCTTCAGTCG |
| | ATCCCT | | CATTTTCATGGAGGGCCAC |
| Tg-rs279844F | CAAGATGCTACGCTTCAGTCTTGCCATGTTTGTC | Tg-rs279844R | CAAGATGCTACGCTTCAGTCA |
| | ACAGGT | | CCTTGGTTTCTTGATTATGTT |
| | | | GAT |
| Tg-rs1058083F | CAAGATGCTACGCTTCAGTCTGAATCCTCCCCCA | Tg-rs1058083R | CAAGATGCTACGCTTCAGTCT |
| | AGCTG | | TCTCCTCTTCCTGGGCTGA |
| Tg-rs2032597F | CAAGATGCTACGCTTCAGTCGCACATTAAATGGG | Tg-rs2032597R | CAAGATGCTACGCTTCAGTCG |
| | TTCCAG | | AAATACGAAGGACACAAAACC |
| | | | TC |
| Tg-rs2032631F | CAAGATGCTACGCTTCAGTCTGTTGCTGGCAAGA | Tg-rs2032631R | CAAGATGCTACGCTTCAGTCT |
| | CACTTC | | GCCTTTGCTACAACTCTCCT |
| Tg-rs2272998F | CAAGATGCTACGCTTCAGTCTAGCCCCACGTCAC | Tg-rs2272998R | CAAGATGCTACGCTTCAGTCT |
| | TTCAG | | TCTTGGAAGGTGGTCCTGG |
| Tg-rs12997453F | CAAGATGCTACGCTTCAGTCAGGACCTGTAAGAG | Tg-rs12997453R | CAAGATGCTACGCTTCAGTCT |
| | TCTGTGATT | | GTATCCCAGGTTCAATGACTG |
| | | | T |
| Tg-rs214955F | CAAGATGCTACGCTTCAGTCACACCCTTACCTGT | Tg-rs214955R | CAAGATGCTACGCTTCAGTCG |
| | ATTTTCTGA | | TGCACATTCTAAGAACTGGTG |
| | | | AT |
| Tg-rs13134862F | CAAGATGCTACGCTTCAGTCTGCTTACAGTGATT | Tg-rs13134862R | CAAGATGCTACGCTTCAGTCA |
| | CTTGCCT | | GTCTTTTGCACCAAGTCTTTT |
| | | | T |
| Tg-rs1410059F | CAAGATGCTACGCTTCAGTCGGAAGATGCTTGAA | Tg-rs1410059R | CAAGATGCTACGCTTCAGTCA |
| | CTCCCCA | | CATCAAAGCTGGGAACCG |
| Tag | CAAGATGCTACGCTTCAGTC | | |

### Pre-Amplification

The primer mix used contained 2 μM of each tagged site-specific pre-amplification primer and 10 μM of the Tag primer (containing the universal tag sequence). Each reaction was 10 μL containing 5 μL of the 2×PCR multiplex mix (Qiagen Catalog #206143), 1 μL of the primer mix, 3 μL of water, and 1 μL of DNA template (˜0.1 ng DNA). The reactions were run in the Mastercycler of Eppendorf. The PCR program was composed of 95° C. for 15 minutes followed by 5 cycles of 94° C. for 30 seconds; 60° C. for 90 seconds; and 72° C. for 40 seconds, and then 15 cycles of 94° C. for thirty seconds; 55° C. for 60 seconds; and 72° C. for 40 seconds; followed by a hold at 4° C.

### Genotyping the Pre-Amplified Samples

A first experiment was performed to see if the target sequence for each SNP was amplified in one reaction effectively. Three primer mixes were used in this experiment: PM1 contains all primers for all 16 SNPs, PM2 contains primers for all of the first 8 SNPs in Table 1; and PM3 contains primers for all of the last 8 SNPs in Table 1. A control DNA was diluted from 2 ng/μL to 0.125 ng/μL in a 2-fold dilution series. The diluted DNA samples were amplified with the three primer mixes as described above. The quality of the PCR products was evaluated using 2% agarose gel electrophoresis. All of the PCR reactions produced 250 base pair to 300 base pair products. The PCR products of the sample with the lowest DNA template concentration (0.125 ng/μL) were diluted 10-fold and 40-fold with nuclease free water and genotyped with the 9 SNPs (the first 9 SNPs in Table 1). The same DNA sample with a concentration of 5 ng/μL was run as a positive control and a concentration of 0.125 ng/μL was run as the control for the background templates without amplification. The results of the Ct value for the two alleles of each marker are summarized in Table 9. All 9 SNPs were amplified in the reactions with the PM1 primer mix. The Ct value difference between the pre-amplified sample (PM1-10 and PM1-40) and the non-amplified control (0.125 ng/μL control) averages around 27 equivalents to about a 2²⁷-fold increase of the template concentration in the sample with pre-amplification. This amount of DNA template is very likely to produce good quality genotype data using the TaqMan® OpenArray® technology (Life Technologies).

A second experiment was performed to see if pre-amplification of the urine DNA extracts could generate correct genotyping results. As described above, we have tested the urine sample DNA extracts on a genotyping TaqMan® OpenArray® from Life Technologies. Out of the 32 urine samples tested, over 88% failed to give genotyping results for all 32 markers. Among them, 11 urine DNA extracts failed to determine a genotype for most SNPs on the array. These 11 samples were selected for this experiment. First, the samples were diluted 10-fold with water to reduce the concentration of potential inhibitors. The diluted samples were then amplified with the PM1 primer mix. The PCR products were diluted by 50-fold and the genotype of 9 SNPs (the first 9 listed in Table 1) were tested. The genotype results were then compared with the genotyping results of the corresponding buccal cell samples (Table 10). Once of the 11 samples failed to generate useful data across the board (U-13M). Out of the remaining 10 samples, 8 gave genotyping results that perfectly matched with that of their corresponding buccal cell samples. Two samples, U-38M and U-40M, had one genotype call discrepancy. As a result, out of the 90 assays performed, 88 were correct (98% accuracy).

The data in Table 10 indicate that the pre-amplification method developed will likely generate good quality DNA template for genotyping any combination of SNPs described herein using TaqMan® OpenArray® technology.

TABLE 9
Ct Values of Allele-Specific Amplification of the 9 SNPs for Control and Pre-Amplified Samples

| | | 5 ng Control | | 0.125 ng Control | | PM1- 10 fold | | PM1- 40 fold | |
|---|---|---|---|---|---|---|---|---|---|
| Sample Name | NCBI SNP | Allele 1 | Allele2 | Allele 1 | Allele2 | Allele 1 | Allele2 | Allele 1 | Allele2 |
| VGTX0004 | rs13182883 | NoCall | 25.0 | NoCall | 30.8 | NoCall | 3.5 | NoCall | 5.3 |
| VGTX0004 | rs560681 | 25.2 | 24.7 | 30.8 | 28.3 | 4.2 | 7.1 | 5.9 | 8.2 |
| VGTX0004 | rs740598 | 26.0 | NoCall | 31.7 | NoCall | 3.8 | NoCall | 6.2 | NoCall |
| VGTX0004 | rs1358856 | 27.6 | 27.3 | 33.4 | 33.1 | 6.8 | 7.1 | 9.3 | 9.3 |
| VGTX0004 | rs9951171 | 26.5 | 26.1 | 31.8 | 31.1 | 3.8 | 3.3 | 6.1 | 5.4 |
| VGTX0004 | rs7520386 | 24.9 | 24.8 | 30.4 | 30.5 | NoCall | 2.9 | 4.7 | 5.1 |
| VGTX0004 | rs13218440 | 26.6 | 26.4 | 31.7 | 31.6 | 3.4 | 3.7 | 5.3 | 5.7 |
| VGTX0004 | rs279844 | NoCall | 27.4 | NoCall | 33.5 | 9.6 | 5.8 | 8.6 | 7.5 |
| VGTX0004 | rs1058083 | NoCall | 25.7 | NoCall | 31.1 | NoCall | 4.8 | NoCall | 6.8 |
| | | PM2- 10 fold | | PM2- 40 fold | | PM3- 10 fold | | PM3- 40 fold | |
| Sample Name | NCBI SNP | Allele 1 | Allele2 | Allele 1 | Allele2 | Allele 1 | Allele2 | Allele 1 | Allele2 |
| VGTX0004 | rs13182883 | NoCall | 3.3 | NoCall | 4.9 | NoCall | 36.9 | NoCall | 33.5 |
| VGTX0004 | rs560681 | 3.7 | 7.3 | 5.3 | 7.0 | 37.6 | NoCall | 33.8 | NoCall |
| VGTX0004 | rs740598 | 2.9 | NoCall | 5.1 | NoCall | 36.3 | NoCall | 35.1 | NoCall |
| VGTX0004 | rs1358856 | 5.8 | 6.1 | 8.3 | 8.3 | 37.2 | NoCall | 36.5 | 36.1 |
| VGTX0004 | rs9951171 | 3.4 | 2.7 | 5.4 | 4.5 | 37.6 | 36.1 | 34.5 | 33.8 |
| VGTX0004 | rs7520386 | 3.0 | 2.6 | 4.7 | 4.3 | 33.1 | 33.3 | 32.7 | 32.8 |
| VGTX0004 | rs13218440 | 3.1 | 2.8 | 5.1 | 4.6 | 36.1 | 35.6 | 33.3 | 33.8 |
| VGTX0004 | rs279844 | 8.6 | 5.2 | 7.7 | 6.9 | NoCall | 39.7 | NoCall | 35.9 |
| VGTX0004 | rs1058083 | NoCall | NoCall | NoCall | NoCall | NoCall | 4.2 | NoCall | 6.8 |

5 ng Control: The positive control.
0.125 ng Control: The template used for pre-amplification. It controls for the background template without amplification.
PM1, PM2, and PM3: The three primer mixes used for pre-amplification.
-10: 10-fold dilution.
-40: 40-fold dilution.

TABLE 10
Comparison of the Genotyping Results from the Pre-Amplified Urine
DNA Extracts and Buccal Cell DNA Extracts of 10 Individuals

| | pre-amp | | Buccal | | pre-amp | | Buccal | |
|---|---|---|---|---|---|---|---|---|
| | Urine | Result | Cell | Result | Urine | Result | Cell | Result |
| Assay ID | Sample | Call | Sample | Call | Sample | Call | Sample | Call |
| C____342791_10 | U-22M | A/G | BC-22M | A/G | U-34M | A/G | BC-34M | A/G |
| C___1006721_1_ | U-22M | A/A | BC-22M | A/A | U-34M | A/G | BC-34M | A/G |
| C___1371205_10 | U-22M | A/A | BC-22M | A/A | U-34M | G/G | BC-34M | G/G |
| C___1619935_1_ | U-22M | G/G | BC-22M | G/G | U-34M | G/G | BC-34M | G/G |
| C___2140539_10 | U-22M | A/A | BC-22M | A/A | U-34M | C/C | BC-34M | C/C |
| C___2556113_10 | U-22M | G/G | BC-22M | G/G | U-34M | G/G | BC-34M | G/G |
| C___3254784_10 | U-22M | A/G | BC-22M | A/G | U-34M | A/A | BC-34M | A/A |
| C___8263011_10 | U-22M | A/T | BC-22M | A/T | U-34M | A/A | BC-34M | A/A |
| C___9371416_10 | U-22M | A/G | BC-22M | A/G | U-34M | G/G | BC-34M | G/G |
| C____342791_10 | U-23F | A/A | BC-23F | A/A | U-38M* | G/G | BC-38M | A/G |
| C___1006721_1_ | U-23F | A/A | BC-23F | A/A | U-38M | A/A | BC-38M | A/A |
| C___1371205_10 | U-23F | A/G | BC-23F | A/G | U-38M | G/G | BC-38M | G/G |
| C___1619935_1_ | U-23F | A/A | BC-23F | A/A | U-38M | G/G | BC-38M | G/G |
| C___2140539_10 | U-23F | A/A | BC-23F | A/A | U-38M | A/C | BC-38M | A/C |
| C___2556113_10 | U-23F | G/G | BC-23F | G/G | U-38M | A/G | BC-38M | A/G |
| C___3254784_10 | U-23F | A/A | BC-23F | A/A | U-38M | G/G | BC-38M | A/G |
| C___8263011_10 | U-23F | A/A | BC-23F | A/A | U-38M | A/A | BC-38M | A/A |
| C___9371416_10 | U-23F | A/A | BC-23F | A/A | U-38M | A/G | BC-38M | A/G |
| C____342791_10 | U-28F | G/G | BC-28F | G/G | U-39M | A/A | BC-39M | A/A |
| C___1006721_1_ | U-28F | A/G | BC-28F | A/G | U-39M | G/G | BC-39M | G/G |
| C___1371205_10 | U-28F | A/A | BC-28F | A/A | U-39M | A/G | BC-39M | A/G |
| C___1619935_1_ | U-28F | G/G | BC-28F | G/G | U-39M | A/G | BC-39M | A/G |
| C___2140539_10 | U-28F | A/A | BC-28F | A/A | U-39M | A/C | BC-39M | A/C |
| C___2556113_10 | U-28F | G/G | BC-28F | G/G | U-39M | A/A | BC-39M | A/A |
| C___3254784_10 | U-28F | A/A | BC-28F | A/A | U-39M | G/G | BC-39M | G/G |
| C___8263011_10 | U-28F | A/T | BC-28F | A/T | U-39M | T/T | BC-39M | T/T |
| C___9371416_10 | U-28F | A/A | BC-28F | A/A | U-39M | A/G | BC-39M | A/G |
| C____342791_10 | U-30F | A/A | BC-30F | A/A | U-40M | G/G | BC-40M | G/G |
| C___1006721_1_ | U-30F | A/A | BC-30F | A/A | U-40M | A/A | BC-40M | A/A |
| C___1371205_10 | U-30F | A/G | BC-30F | A/G | U-40M | G/G | BC-40M | G/G |
| C___1619935_1_ | U-30F | A/G | BC-30F | A/G | U-40M | A/A | BC-40M | A/A |
| C___2140539_10 | U-30F | A/C | BC-30F | A/C | U-40M | A/A | BC-40M | A/A |
| C___2556113_10 | U-30F | G/G | BC-30F | G/G | U-40M | A/G | BC-40M | A/G |
| C___3254784_10 | U-30F | G/G | BC-30F | G/G | U-40M* | A/A | BC-40M | A/G |
| C___8263011_10 | U-30F | A/A | BC-30F | A/A | U-40M | A/T | BC-40M | A/T |
| C___9371416_10 | U-30F | G/G | BC-30F | G/G | U-40M | G/G | BC-40M | A/G |
| C____342791_10 | U-31F | A/A | BC-31F | A/A | VGTX0038 | A/G | BC-34M | A/G |
| C___1006721_1_ | U-31F | A/G | BC-31F | A/G | VGTX0038 | A/G | BC-34M | A/G |
| C___1371205_10 | U-31F | A/G | BC-31F | A/G | VGTX0038 | G/G | BC-34M | G/G |
| C___1619935_1_ | U-31F | A/A | BC-31F | A/A | VGTX0038 | G/G | BC-34M | G/G |
| C___2140539_10 | U-31F | A/C | BC-31F | A/C | VGTX0038 | C/C | BC-34M | C/C |
| C___2556113_10 | U-31F | A/G | BC-31F | A/G | VGTX0038 | G/G | BC-34M | G/G |
| C___3254784_10 | U-31F | A/A | BC-31F | A/A | VGTX0038 | A/A | BC-34M | A/A |
| C___8263011_10 | U-31F | A/T | BC-31F | A/T | VGTX0038 | A/A | BC-34M | A/A |
| C___9371416_10 | U-31F | A/G | BC-31F | A/G | VGTX0038 | G/G | BC-34M | G/G |
| C____342791_10 | U-33F | A/A | BC-33F | A/A | VGTX0038 is the | | | |
| C___1006721_1_ | U-33F | A/A | BC-33F | A/A | control | | | |
| C___1371205_10 | U-33F | G/G | BC-33F | G/G | | | | |
| C___1619935_1_ | U-33F | A/A | BC-33F | A/A | | | | |
| C___2140539_10 | U-33F | A/C | BC-33F | A/C | | | | |
| C___2556113_10 | U-33F | G/G | BC-33F | G/G | | | | |
| C___3254784_10 | U-33F | A/A | BC-33F | A/A | | | | |
| C___8263011_10 | U-33F | T/T | BC-33F | T/T | | | | |
| C___9371416_10 | U-33F | G/G | BC-33F | G/G | | | | |

### Example 2. Multicenter Test Trial

A multicenter test trial is conducted to evaluate the rate of urine sample substitution in the clinic. Three types of medical centers are included in this study: addiction recovery outpatient centers, pain management clinics, and family practice health care facilities. Known matched and mismatched samples (urine samples and buccal cell samples) are introduced at each site as controls. The sample size required for reliable results with 95% confidence interval less than 5% is shown in Table 11. The methods in this assay are as described above (e.g., pre-amplification of the target for a combination of SNPs as described herein and genotyping using TaqManR OpenArray® technology from Life Technologies).

The 95% confidence interval is different for different sample sizes based on the estimated percentage rate of buccal cell sample/urine sample mismatch in the population. Without knowing the true mismatch rate in the population, 1000 samples are used to keep the 95% confidence interval below 3.5%.

TABLE 11
Multi-Site Study Population

| | 95% confidence interval | | | | | |
|---|---|---|---|---|---|---|
| % miss-match | 500* | 600* | 800* | 1000* | 1500* | 2000* |
| 10.0% | 2.63 | 2.4 | 2.08 | 1.86 | 1.25 | 1.31 |
| 15.0% | 3.13 | 2.86 | 2.47 | 2.21 | 1.81 | 1.56 |
| 20.0% | 3.51 | 3.2 | 2.77 | 2.48 | 2.02 | 1.75 |
| 25.0% | 3.80 | 3.46 | 3 | 2.68 | 2.19 | 1.9 |
| 30.0% | 4.02 | 3.67 | 3.18 | 2.84 | 2.31 | 2.01 |
| 40.0% | 4.29 | 3.92 | 3.39 | 3.04 | 2.48 | 2.15 |

### Example 3. Detection of Statherin in Urine Samples

Another possible way a subject may adulterate his/her urine sample is to place some of his/her saliva into synthetic urine or a different subject's urine. Because there is a lot more cells in one's saliva than in urine, the genotype of the saliva donor can overshadow that of the urine donor. Detection of saliva in the urine sample will confirm that the urine sample has been adulterated. In this set of experiments, a method for detecting the presence of a unique low-molecular weight saliva phosphoprotein (statherin) in a urine sample was developed and its sensitivity tested. Statherin is known to be uniquely present in saliva as it is secreted from the parotid gland.

### Materials

Materials used for the test:
Statherin Antibody (N-16), Santa Cruz Biotechnology, Inc. (Catalog No. sc-28112);
Statherin (N-16)P, Santa Cruz Biotechnology, Inc. (Catalog No. sc-28112-P);
Rabbit Anti-Goat IgG-AP, Santa Cruz Biotechnology, Inc. (Catalog No. sc-2771);
Para-nitrophenylphosphate (PNPP), Santa Cruz Biotechnology, Inc. (Catalog No. sc-3720);
PNPP substrate buffer, Santa Cruz Biotechnology, Inc. (Catalog No. sc-296099);
0.05 M bicarbonate buffer, Sigma (Catalog No. C3041-50CAP);
Bovine serum albumin, Sigma (Catalog No. A9418-5G); and
Nunc MaxiSorp® flat-bottom 96-well plate, Affimetrix eBioscience (Catalog No. 44-2404-21).

The buffers used include a 50 mM bicarbonate buffer (made by dissolving the contents of one capsule in 100 mL deionized water). The content of one capsule yields 100 mL of 0.05 M carbonate-bicarbonate buffer, pH 9.6 at 25° C. The 1×PBS buffer used was prepared by diluting 10×PBS in HPLC water. The PBST buffer was made by adding 0.05% Tween 20 into 1×PBS. An additional buffer of 1% bovine serum albumin in 1×PBS was also prepared.

Dilutions of the statherin antibody (N-16) (1:50, 1:200, and 1:500) were made with 1% bovine serum albumin in 1×PBS. Dilutions of the detection rabbit anti-goat IgG-AP (1:1000) was made in 1% BSA in 1×PBS.

### Methods and Results

A first experiment was performed to optimize the reaction conditions to determine: the proper ratio of the urine sample to the 50 mM bicarbonate buffer for antigen coating and the best concentration of anti-statherin antibody to use in the assay. In this experiment, a saliva sample was collected from an individual and diluted 1:10, 1:50, or 1:100 with 50 mM bicarbonate buffer. Fifty μL of each diluted saliva sample was pipetted into a well of Nunc MaxiSorp® flat-bottom 96-well plates. Three samples were made for each dilution. Three wells with only the 50 mM bicarbonate buffer were used as a negative control. The plate was incubated at 4° C. overnight covered with the parafilm to coat statherin in the sample onto the well surface. After coating, the nonspecific binding sites on the well surface were blocked by adding 200 μL of 1% bovine serum albumin in PBS to each well and incubating the plate at room temperature for 2 hours. The plate was washed once with 250 μL of 1×PBS followed by incubation with 50 μL of the diluted anti-statherin antibody at 37° C. for 1 hour. The sample plate layout is presented is shown in Table 12. After incubation with the anti-statherin antibody, the plate was washed three times with 250 μL PBST with 2 minutes incubation between the washes. After washing, 50 μL of the 1:1000 diluted rabbit anti-goat IgG-AP in 1% bovine serum albumin in PBS was added to each well and the plate was incubated at 37° C. for 1 hour followed by three washes with 250 μL PBST at room temperature with 2 minutes incubation between the washes. The wells were rinsed once with 100 μL of PNPP substrate buffer and incubated with 50 μL of the PNPP substrate (1 mg/mL in PNPP substrate buffer) at room temperature for 20 minutes. The absorbance of the plate at 405 nm was measured, and the absorbance of the plate at 490 nm was used as a reference.

TABLE 12
Plate Layout of Samples

| Anti-Statherin | 1 | 2 | 3 | 4 |
|---|---|---|---|---|
| 1:50 AB | 1:10 Saliva | 1:50 Saliva | 1:100 Saliva | NTC |
| 1:100 AB | 1:10 Saliva | 1:50 Saliva | 1:100 Saliva | NTC |
| 1:500 AB | 1:10 Saliva | 1:50 Saliva | 1:100 Saliva | NTC |

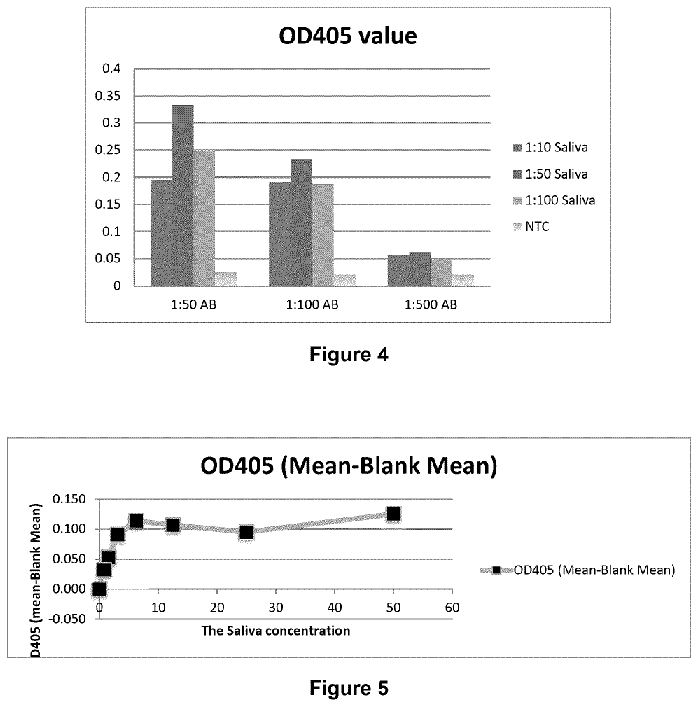

The results of this experiment are shown in FIG. 4. The highest absorbance at 405 nm was obtained with the 1:50 diluted saliva sample stained by the anti-statherin antibody at the 1:50 dilution. Although the absorbance at 405 nm of the anti-statherin antibody 1:50 dilution is higher than that observed for the anti-statherin 1:100 dilution, the difference is not significant. Since the higher dilution reduces the reagent cost when testing a large number of samples, the 1:100 dilution of anti-statherin antibody was chosen as the concentration for the primary antibody. The best ratio of the saliva to the coding buffer is 1:50.

A second experiment was performed to see if statherin present in a urine sample can be detected and if so, to determine what the lowest detection limit is for statherin in a urine sample. In this experiment, 100 μL of saliva collected from a subject was mixed with 100 μL of urine from the same individual. Two-fold serial dilutions of this starting mixed sample (Sample 1) were made using urine from the same individual. The dilutions of the mixed samples are shown in Table 13.

TABLE 13
Dilutions of the Saliva and Urine Mixed Sample

| | Saliva | Urine | Final Saliva |
|---|---|---|---|
| Sample ID | (uL) | (uL) | Con (uL) |
| 1 | 100 | 100 | 50 |
| 2 | 50 | 100 | 25 |
| 3 | 25 | 100 | 12.5 |
| 4 | 12.5 | 100 | 6.25 |
| 5 | 6.25 | 100 | 3.125 |
| 6 | 3.125 | 100 | 1.5625 |
| 7 | 1.625 | 100 | 0.78125 |
| 8 | 0 | 100 | 0 |

The diluted mixed samples 1-8 were then diluted 1:40 in the 50 mM bicarbonate buffer. The diluted samples were then used to coat the plate and tested following the same protocol as described above in this Example. In this experiment, the anti-statherin antibody was diluted 1:100 in 1% bovine serum albumin in PBS.

The results of this experiment are presented in Table 14. The positive controls were made by mixing 10 μL of the diluted rabbit anti-goat IgG-AP with the substrate PNPP. Each sample was run in triplicate, the value of the absorbance at 405 nM of each reaction was obtained using a plate reader. The mean and standard deviation of each sample was calculated using an Excel program. The standard deviation of the saliva free urine is 0.00208167. Therefore, the limit of detection (LOD) is 0.01 (3× the standard deviation of the blank) and the limit of quantification (LOQ) is 0.021 (10× the standard deviation of the blank). The mean OD405 value of sample 7 (with the mean blank OD405 subtracted) is 0.032 which is larger than 0.021, the LOQ of the assay. These data show that statherin can be detected when as little as 0.8 μL of saliva is present in 100 μL of urine (<1%). Normally, each individual is asked to provide 30 mL of urine for drug testing. It is likely that a subject trying to adulterate the urine sample with saliva would spit about 500 μL to 1 mL of saliva into the sample to get enough of their own cells into the urine sample (e.g., a synthetic urine). The estimated final concentration in a 30 mL sample would range from 1.67 μL−3.3 μL saliva per 100 μL of urine. This concentration falls into the test sensitivity range of the assay.

TABLE 14
The OD405 Value of the ELISA test of Statherin
in a 2-fold Serial Dilutions of Saliva in Urine

| | OD405 | | | | | | |
|---|---|---|---|---|---|---|---|
| Saliva Concentration | Sample | | | | | | Mean - Blank |
| (uL/100 uL) | ID | R1 | R2 | R3 | Mean | STDEV | mean |
| 50 | 1 | 0.171 | 0.151 | 0.144 | 0.155 | 0.0140119 | 0.125 |
| 25 | 2 | 0.147 | 0.114 | 0.114 | 0.125 | 0.01905256 | 0.095 |
| 12.5 | 3 | 0.161 | 0.129 | 0.120 | 0.137 | 0.0215484 | 0.107 |
| 6.25 | 4 | 0.160 | 0.140 | 0.131 | 0.144 | 0.01484363 | 0.114 |
| 3.125 | 5 | 0.132 | 0.117 | 0.114 | 0.121 | 0.00964365 | 0.091 |
| 1.5625 | 6 | 0.091 | 0.083 | 0.075 | 0.083 | 0.008 | 0.053 |
| 0.78125 | 7 | 0.069 | 0.060 | 0.057 | 0.062 | 0.006245 | 0.032 |
| 0 | 8 | 0.028 | 0.029 | 0.032 | 0.030 | 0.00208167 | 0.000 |
| NTC (buffer) | 9 | 0.022 | 0.023 | 0.028 | 0.024 | 0.00321455 | |
| Positive | | 2.472 | 2.552 | 2.367 | 2.464 | 0.0927811 | |

There were two negative controls: one was a urine sample not containing saliva, and one was buffer with no added saliva. The OD405 of each sample (with the mean blank OD405 subtracted) is shown in FIG. 5. The data in FIG. 5 show a linear increase in OD405 values with increasing saliva concentrations (ranging from 0-6.25 μL of saliva per 100 μL urine). The signal plateaued for the three higher saliva concentration samples. These data show that the assay is able to detect saliva in urine samples with a high sensitivity.

A further experiment was performed to see if the assay can detect the presence of statherin in a mixed sample of saliva and urine from the same subject (SP-U) and a mixed sample of a buccal cells and urine from the same subject (SB-U). The urine samples were collected from two individuals. Two 30-mL urine aliquots were made from each collected urine sample. The first urine aliquot was mixed with the individual's spit (˜500 μL). For the second aliquot, a buccal swab was collected from the individual and mixed with the urine via vigorous stirring. A saliva-free urine sample was used as a negative control and a pure saliva sample was run as a positive control. The samples were diluted 1:40 in the 50 mM bicarbonate buffer and tested as described above in this Example. The plate layout is shown in Table 15.

TABLE 15
Plate Layout of Experiment Testing Mixed Saliva and
Urine Samples and Mixed Buccal Cell and Urine Samples

| | Individual A | | | | Individual B | | | |
|---|---|---|---|---|---|---|---|---|
| | 1 | 2 | 3 | 4 | 5 | 6 | 7 | 8 |
| 1 | A-Saliva | A-SP-U | A-SB-U | A-U | B-Saliva | B-SP-U | B-SB-U | B-U |
| 2 | A-Saliva | A-SP-U | A-SB-U | A-U | B-Saliva | B-SP-U | B-SB-U | B-U |
| 3 | A-Saliva | A-SP-U | A-SB-U | A-U | B-Saliva | B-SP-U | B-SB-U | B-U |

The data from this experiment are summarized in Table 16. As shown in Table 15, individual A's saliva contains 3.7-fold higher statherin than individual B's saliva indicating individual variability in statherin concentration in their salivas (OD 405 1.222 compared to 0.336). The OD405 values of the mixed saliva and urine samples and the mixed buccal cell and urine samples for both individuals was much higher than that of the urine only control samples (A-U and B-U), indicating the presence of statherin in the mixed samples. The standard deviation of the three replicates were very small, ranging from 0.012 to 0.053, which demonstrates that high precision of the assay system.

TABLE 16
OD405 values Mixed Saliva and Urine Samples, Mixed
Buccal Cell and Urine Samples, and Controls
OD405

| Sample | R1 | R2 | R3 | Mean | STDEV |
|---|---|---|---|---|---|
| A-Saliva | 1.214 | 1.216 | 1.236 | 1.222 | 0.012 |
| A-SP-U | 0.207 | 0.229 | 0.236 | 0.224 | 0.015 |
| A-SB-U | 0.331 | 0.287 | 0.291 | 0.303 | 0.024 |
| A-U | 0.036 | 0.035 | 0.033 | 0.035 | 0.002 |
| B-Saliva | 0.325 | 0.289 | 0.394 | 0.336 | 0.053 |
| B-SP-U | 0.171 | 0.168 | 0.165 | 0.168 | 0.003 |
| B-SB-U | 0.318 | 0.311 | 0.289 | 0.306 | 0.015 |
| B-U | 0.062 | 0.041 | 0.064 | 0.056 | 0.013 |

In summary, the data in this Example show that the statherin assay is able to robustly and sensitively detect statherin in urine samples. Less than 1% of the saliva mixed in a urine sample can be detected by the assay. The data show that the assay can detect statherin when saliva was mixed with urine either by spitting into the urine or stirring a buccal cell swab in the urine.

### Example 4. Open Array Assay Validation

The data described above show that urine samples can be genotyped for 11 SNPs using a TaqMan real-time PCR genotyping method in the 384 well format with 100% accuracy and specificity. In order to scale-up the capacity, an Open Array was custom designed with 16 assays for the targeted highly polymorphic SNPs shown in Table 1.

An important element for the success of the Open Array assay to genotype SNPs is to have a good quantity and quality of DNA template for the test. DNA extracts from 32 urine samples were tested on a pre-made DNA identification Open Array with 32 assays from Life Technologies (Catalog #4475386). More than 88% of the samples did not produce genotype results for all assays. This result indicates that the DNA extracts of the urine samples are not good for use in an Open Array test and that the pre-amplification step described in Example 1 can be used to provide sufficient quantity and quality of DNA template for the Open Array assay. In this set of experiments, the accuracy and reproducibility of the Open Array assay for genotyping 16 SNPs was determined using the pre-amplified PCR products generated from urine samples.

### Materials and Methods

### Reagents and Instruments

1) The 2×Open Array TaqMan Genotyping Master Mix from Life Technologies (Cat #1307038).
2) The nuclease-free water from Fisher (Cat #AXH44096).
3) The pre-amplification primers were designed in house and synthesized by IDT Inc.
4) Qiagen Multiplex PCR Kit (100) (Cat #206143).
5) QuantStudio™ 12K Flex System from Life Technologies.
6) Mastercycler nexus from Eppendorf.
7) Centrifuge 5810R from Eppendorf.

### Experimental Samples

The same urine and buccal cell DNA extracts from Example 2 were used for this test. The sample ID and DNA concentration are shown in Table 17.

TABLE 17
The DNA Samples Used for the Open Array Test.

| | Sample | DNA | Buccal | DNA |
|---|---|---|---|---|
| # | ID | (ng/uL) | Cells | (ng/uL) |
| 1 | U-01F-321 | 7.5 | BC-01F-0321 | 26.2 |
| 2 | U-02F-321 | 6.2 | BC-02F-0321 | 12.3 |
| 3 | U-04F-321 | 1440.7* | BC-04F-0321 | 47.8 |
| 4 | U-05F-321 | 3.1 | BC-05M-0321 | 12.4 |
| 5 | U-06F-321 | 1174.4* | BC-06F-0321 | 36.7 |
| 6 | U-07F-321 | 656.2* | BC-07F-0321 | 54.3 |
| 7 | U-08F-321 | 23.6 | BC-08F-0321 | 36.4 |
| 8 | U-10F-321 | 0.7** | BC-10M-321 | 89.7 |
| 9 | U-12F-321 | 0.1** | BC-12F-321 | 32.9 |
| 10 | U-14F-321 | 3 | BC-14F-321 | 42.3 |
| 11 | U-16F-321 | 10.1 | BC-16F-321 | 74.8 |
| 12 | U-17F-321 | 8.8 | BC-17F-321 | 55.4 |
| 13 | U-18M-321 | −0.8** | BC-18M-321 | 119.2 |
| 14 | U-19M-321 | 1306* | BC-19M-321 | 95 |
| 15 | U-21M-321 | −1.6 | BC-21M-321 | 149.9 |
| 16 | U-22M-321 | 35.6 | BC-22M-321 | 237.2 |
| 17 | U-23F-321 | 1212.4* | BC-23F-321 | 29.3 |
| 18 | U-24F-321 | 78.7 | BC-24F-321 | 48 |
| 19 | U-25F-321 | 80.6 | BC-25F-321 | 75 |
| 20 | U-27F-321 | 5.8 | BC-27F-321 | 80.4 |
| 21 | U-28F-321 | 813.7* | BC-28F-321 | 15.1 |
| 22 | U-29F-321 | 23 | BC-29F-321 | 85.3 |
| 23 | U-30F-321 | 2207.5* | BC-30F-321 | 163 |
| 24 | U-31F-321 | 1429.6* | BC-31F-321 | 68.7 |
| 25 | U-32M-321 | 32.6 | BC-32M-321 | 55.5 |
| 26 | U-33F-321 | 2.7 | BC-33F-321 | 54.6 |
| 27 | U-34M-321 | −2.8** | BC-34M-321 | 117.6 |
| 28 | U-37F-321 | 2.2 | BC-37F-321 | 72.1 |
| 29 | U-38M-321 | 992.8* | BC-38M-321 | 38.4 |
| 30 | U-39M-321 | −2.8** | BC-39M-321 | 125.6 |
| 31 | U-40M-321 | 6.7 | BC-40M-321 | 78.1 |
| 32 | U-44M-321 | −2.8** | BC-44M-321 | 58.5 |
| 33 | U-47F-321 | 26 | BC-47F-321 | 90.6 |

*Sample contains unknown molecules causing exceptionally high OD260 value. Therefore DNA concentration of these samples is not accurate.
**Samples with too little DNA to be measured.

### Pre-Amplification Primer Mix

The composition of the pre-amplification primer mix is shown in Table 18 below. The sequence of each pre-amplification primer is shown in Table 8.

### Pre-Amplfication

The urine DNA extracts (prepared as described above) were diluted 10-fold with nuclease-free water. The diluted samples were then used as template for pre-amplification in

TABLE 18
Pre-Amplification Primer Mix

| Forward | Qut (100 | Reverse | Qut (100 | Final con |
|---|---|---|---|---|
| primer | pM/uL) | primer | pM/uL) | (pM/uL) |
| Tg-rs13182883F | 4 | Tg-rs13182883R | 4 | 2 |
| Tg-rs560681F | 4 | Tg-rs560681R | 4 | 2 |
| Tg-rs740598F | 4 | Tg-rs740598R | 4 | 2 |
| Tg-rs1358856F | 4 | Tg-rs1358856R | 4 | 2 |
| Tg-rs9951171F | 4 | Tg-rs9951171R | 4 | 2 |
| Tg-rs7520386F | 4 | Tg-rs7520386R | 4 | 2 |
| Tg-rs13218440F | 4 | Tg-rs13218440R | 4 | 2 |
| Tg-rs279844F | 4 | Tg-rs279844R | 4 | 2 |
| Tg-rs1058083F | 4 | Tg-rs1058083R | 4 | 2 |
| Tg-rs2032597F | 4 | Tg-rs2032597R | 4 | 2 |
| Tg-rs2032631F | 4 | Tg-rs2032631R | 4 | 2 |
| Tg-rs2272998F | 4 | Tg-rs2272998R | 4 | 2 |
| Tg-rs12997453F | 4 | Tg-rs12997453R | 4 | 2 |
| Tg-rs214955F | 4 | Tg-rs214955R | 4 | 2 |
| Tg-rs13134862F | 4 | Tg-rs13134862R | 4 | 2 |
| Tg-rs1410059F | 4 | Tg-rs1410059R | 4 | 2 |
| Tag | 20 | | | 10 |
| H2O | 52 | | | |

a 10-μL PCR reaction containing: 2 μL of DNA template, 5 μL of the 2×Qiagen Multiplex PCR mix, 0.25 μL of the primer mix, and 2.75 μL of the nuclease free water. The pre-amplification reactions were run using a PCR program of: 95° C. for 15 minutes; followed by 5 cycles of 94° C. for 30 seconds, 60° C. for 90 seconds, and 72° C. for 40 seconds; followed by 15 cycles of 94° C. for 30 seconds, 55° C. for 60 seconds, and 72° C. for 40 seconds; followed by 1 cycle of 72° C. for 5 minutes and then hold at 4° C. To evaluate the reproducibility of the array, the same set of samples were pre-amplified in 5 separate batches on 5 different days. The PCR products were diluted 30-fold with water for the Open Array test.

Open Array Test

For the Open Array test, 2 μL of the diluted pre-amplification PCR products were mixed with 2 μL of the 2×Open Array TaqMan assay master mix in the sample well. The samples were loaded onto the Open Array using the Open Array Accufill system. The loaded array was then run in QuantStudio 12K Flex Real-time PCR system. A total of three arrays were run for validation. The first array was used to determine if the Open Array can produce accurate genotyping results and to determine the optimal dilution of the PCR products for accurate genotyping. The first batch of pre-amplified products was diluted 30- and 60-fold, respectively. The diluted samples were then tested together with the buccal cell DNA extracts of the same individuals. The samples with 30-fold dilution gave the best results with 98.5% genotype results matched with that of their corresponding buccal cell DNA extracts. Therefore, the 30-fold dilution of PCR products was used as the standard protocol. The same set of urine samples were then pre-amplified in 4 different batches. Samples from batches 1 and 2 were run on the second array in duplicates, and samples from batches 3 and 4 were run on the third array in duplicates. Sample VGTX0004 and VGTX0038 buccal cell extracts were tested 4 times on each array as the control. As the result, each sample was tested in three separate batches for a total of 9 replicates. The genotype data of these replicates were then compared to evaluate the accuracy and reproducibility of these arrays.

### PCR and Sequencing

Out of the 16 arrays loaded on the Open Array, 5 assays have not been validated before. In this study, we validated these assays using Sanger sequencing. For sequencing, 10 DNA samples extracted from urine of 10 individuals were diluted 5-fold with water and used as the template for PCR. The PCR was done in a volume of 25 μL containing 2 μL of DNA template, 12.5 μL of the Qiagen Multiplex PCR mix (Cat #206143), 1 μL of primer mix, and 9.5 μL of nuclease free water. The reactions were carried out in the Thermal cycler using the PCR program of 95° C. for 15 minutes; followed by 40 cycles of 94° C. for 30 seconds, then 57° C. for 1 minute, and then 72° C. for 1 minute; followed by incubation at 72° C. for 6 minutes. After PCR, 5 μL of the reaction was then run on a 1.5% agarose gel to check for the quality of the PCR reaction. After verification that the PCR reaction worked well, 10 μL of each reaction was transferred to a fresh 8 well strip tube and sent to GenQiz, a CLIA certified sequence provider (CLIA ID: 31D2038676) for sequencing with the specific sequencing primer. The sequencing result was then analyzed using free software FinchTV and the SNP genotypes were manually called.

### Results

The first experiment was performed to evaluate if the Open Array can produce correct genotyping results. Sixteen assays were performed using the Open Array, and eleven of the assays have been validated using the 384-well assay. The samples used for Open Array validation have been tested multiple times via real-time and Sanger sequencing. Therefore, the genotype of these samples were known. The additional 5 assays that were not previously validated were sequenced using Sanger Sequencing for 10 selected samples. The sequencing results are compared with the genotyping results from the Open Array. These results are shown in Table 19.

TABLE 19
Sanger Sequencing and Open Array Genotyping Results for the 5 Additional Assays

| | rs [symbol] | | rs [symbol] | | rs [symbol] | | rs [symbol] | | rs [symbol] | |
|---|---|---|---|---|---|---|---|---|---|---|
| Sample ID | Sequence | CA | Sequence | OA | Sequence | OA | Sequence | OA | Sequuence | OA |
| V-01F-0321 | T/T | T/T | T/T | T/T | C/G | C/G | G/G | G/ [symbol] | A/G | A/ [symbol] |
| V-02F-0321 | T/T | T/T | C/T | C/T | C/G | C/G | A/G | A/ [symbol] | A/G | A/ [symbol] |
| V-04F-0321 | T/T | T/T | C/T | C/T | C/G | C/G | A/G | A/ [symbol] | A/G | A/ [symbol] |
| V-06F-0321 | C/T | C/T | C/C | C/C | C/G | C/G | A/G | A/ [symbol] | A/G | A/ [symbol] |
| V-07F-0321 | T/T | T/T | T/T | T/T | C/C | C/G | A/A | A/ [symbol] | A/G | A/ [symbol] |
| V-0 [symbol] F-0321 | C/T | C/T | C/T | C/T | C/G | C/G | A/A | A/ [symbol] | A/G | A/ [symbol] |
| V-12F-0321 | C/T | C/T | C/T | C/T | C/G | C/G | A/A | A/ [symbol] | G/G | G/ [symbol] |
| V-14F-0321 | C/T | C/T | T/T | T/T | G/G | G/G | A/A | A/ [symbol] | A/G | A/ [symbol] |
| V-16F-0321 | C/C | C/C | C/T | C/T | G/G | G/G | A/G | A/ [symbol] | A/G | A/ [symbol] |
| V-17F-0321 | C/T | C/T | C/T | C/T | C/G | C/G | G/G | G/ [symbol] | G/G | G/ [symbol] |

| | | | | | |
|---|---|---|---|---|---|
| [symbol] | 10 | 10 | 10 | 10 | 10 |
| Accuracy | 100% | 100% | 100% | 100% | 100% |

[symbol]

indicates data missing or illegible when filed

As shown in Table 19, all genotypes matched between the Sanger sequencing and Open Array results indicating that the Open Array assay results are accurate for these 5 assays.

The overall accuracy of the Open Array assay for all 16 assays is evaluated by comparison of the genotype results of the 10 DNA extracts of the Open Array with that of expected (the genotype results from Sanger sequencing). The results are summarized in Table 20. All the genotypes of all samples were correctly determined using the Open Array assay (Table 20).

As shown in Table 17, the DNA quality of the urine sample extracts from the 33 individuals was not good with over 16 samples having very little DNA or containing an unknown substance causing exceptionally high OD260 readings. The low amounts of DNA or unknown substance may explain why the samples did not work on the 32 assay DNA array without pre-amplification. In this study, the samples were pre-amplified at the target SNP sites, the PCR products diluted by 30- or 60-fold, and tested on the Open Array (ORB74)

TABLE 20
Open Array Results for 16 Assays of the 10 DNA Extracts

| | | 01F | | 02F | | 04F | | 06F | | 07F | | |
|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] |
| C____ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C____ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| | | 08F | | 12F | | 14F | | 16F | | 17F | | |
| C____ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] _ | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |
| C___ [symbol] _ [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

along with the DNA extracts of buccal cells obtained from the same individuals. The results are summarized in Table 21. There were a total of 33 samples tested. Each sample was tested for 16 assays resulted in a total of 528 assays tested. The number of assays matched between the buccal cell DNA sample and the diluted pre-amplified PCR products of the urine DNA extract is 520 for the 1:30 dilution template and 517 for the 1:60 dilution template, and resulted in an accuracy rate of 98.5% and 97.9%, respectively. These data demonstrate that the pre-amplification method works well to generate good quality DNA templates from urine DNA for genotyping on the high throughput Open Array format.

The same set of samples were PCR pre-amplified in 4 separate batches on 4 different days to evaluate the overall accuracy and reproducibility of the assay. Each sample was then tested in duplicates on the Open Array. Samples from pre-amplification batches 1 and 2 were tested on array ORB75 and samples from pre-amplification bates 3 and 4 were tested on array ORB76. The accuracy for each one of the 16 assays is summarized in Table 22.

TABLE 21
Summary of the Open Array Genotyping Results
of 33 Buccal Cell DNA Samples and Their Corresponding
Pre-Amplified Urine DNA Samples

No of assays matched

| # samples | Total number | 1 to 30 | 1 to 60 |
|---|---|---|---|
| tested | of assays | Diution | Diution |
| 33 | 528 | 520 | 517 |
| Accuracy rate | | 98.5% | 97.9% |

TABLE 22
Open Array Test Accuracy for Each Assay

| | Gene | NCBI | No. | No. | Call | No Miss | |
|---|---|---|---|---|---|---|---|
| Assay ID | Symbol | SNP | Tested | Called | Rate | Calls | Accuracy |
| C____342791_10 | PRDM2 | rs7520386 | 297 | 297 | 100.0% | 3 | 99.0% |
| C___1006721_1_ | LY9 | rs560681 | 297 | 297 | 100.0% | 3 | 99.0% |
| C___1083231_10 | USP9Y | rs2032597 | 297 | 294 | 99.0% | 0 | 100.0% |
| C___1256256_1_ | SASH1 | rs2272998 | 297 | 293 | 98.8% | 0 | 100.0% |
| C___1276208_10 | CERKL | rs12997453 | 297 | 296 | 99.7% | 2 | 99.3% |
| C___1371205_10 | RAB31 | rs9951171 | 297 | 296 | 99.7% | 0 | 100.0% |
| C___1619935_1_ | UBAC2 | rs1058083 | 297 | 293 | 98.7% | 4 | 98.7% |
| C___1880371_10 | RCHY1 | rs13134862 | 297 | 295 | 99.3% | 8 | 97.3% |
| C___2140539_10 | TRDN | rs1358856 | 297 | 293 | 98.7% | 0 | 100.0% |
| C___2414552_10 | KDM5D | rs2032631 | 297 | 292 | 98.3% | 11 | 96.2% |
| C___2515223_10 | SYNE1 | rs214955 | 297 | 293 | 98.7% | 6 | 98.0% |
| C___2556113_10 | SPOCK1 | rs13182883 | 297 | 294 | 99.0% | 9 | 96.9% |
| C___3254784_10 | HSPA12A | rs740598 | 297 | 297 | 100.0% | 0 | 100.0% |
| C___7538108_10 | SORBS1 | rs1410059 | 297 | 296 | 99.7% | 10 | 96.6% |
| C___8263011_10 | GABRA2 | rs279844 | 297 | 291 | 98.0% | 7 | 97.6% |
| C___9371416_10 | HIVEP1 | rs13218440 | 297 | 297 | 100.0% | 5 | 98.3% |

A total of 33 samples were tested across the 16 assays and each sample was tested 9 times. Thus, each assay was tested 297 times. The calling rate (genotyping rate) is calculated as the ratio of the number of tests that made genotype calls to the total number of tests performed. The accuracy is calculated as the ratio of the number of tests that made correct genotype calls to the total number of test made calls. The call rate ranges from 98% to 100%, and the accuracy rate ranges from 96.2% to 100%. Assay C_2414552_30, C-2556113_10, and C_7538108_10 have higher error rate than the others. Therefore special care is required to determine whether a sample is a positive or negative match when mismatch between the buccal cell sample and urine sample is for one of these three assays.

The test reproducibility was evaluated by the rate of genotype agreement between the 9 replicates for all of the assays for each sample. The results are summarized in Table 23. The accuracy is calculated as the ratio of the number of correctly called assays of each sample to the number of assays that generated genotype calls. The reproducibility is calculated as the ratio of the number of correct called assays of each sample to the total number of assays performed. Out of the 33 samples, 30 have accuracy about 97% and reproducibility above 94%. Two samples U-40M and U-44M had the reproducibility around 90% and accuracy around 92%. The detailed genotype results of these two samples are summarized in Table 24.

TABLE 23
Open Array Assay Accuracy and Reproducibility

| | | | # | | | | No. of | No. of | | |
|---|---|---|---|---|---|---|---|---|---|---|
| Urine | # | # | Total | No. of | No. of | Call | Miss | Correct | | |
| Sample | Assay | Replicates | assays | NoCall | Called | Rate | Call | Call | Reproducibility | Accuracy |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U- [symbol] -0321 | [symbol] | [symbol] | [symbol] | [symbol] | [symbol] | [symbol] | [symbol] | [symbol] | [symbol] | [symbol] |
| U-40M-0321 | 16- | 9 | 144 | 6 | 138 | 95.8% | 10 | 128 | 88.9% | 92.8% |
| U-44M-0321 | 16- | 9 | 144 | 2 | 142 | 98.6% | 10 | 132 | 91.7% | 93.0% |
| U- [symbol] -0321 | [symbol] | [symbol] | [symbol] | [symbol] | [symbol] | [symbol] | [symbol] | [symbol] | [symbol] | [symbol] |
| Average | | | | | | [symbol] | | | [symbol] | [symbol] |

[symbol]

indicates data missing or illegible when filed

TABLE 24
Detailed Genotype Results of Sample U-40M and U-44M

| | | NCBI | Sample | [symbol] | [symbol] | | | | [symbol] | | | | Buccal |
|---|---|---|---|---|---|---|---|---|---|---|---|---|---|
| Assay ID | Gene | SNP | ID | Call | A1-B1 | A2-B1 | A1-B2 | A2-B2 | A1-B1 | A2-B1 | A1-B2 | A2-B2 | Cell |
| C_____ [symbol] _ [symbol] | [symbol] | [symbol] | [symbol] | G/G | [symbol] | [symbol] | [symbol] | [symbol] | G/G | G/G | G/G | G/G | [symbol] |
| C___ [symbol] _ [symbol] _ | [symbol] | [symbol] | [symbol] | A/A | [symbol] | [symbol] | [symbol] | [symbol] | A/A | A/A | A/A | A/A | [symbol] |
| C___ [symbol] _ [symbol] | [symbol] | [symbol] | [symbol] | A/A | NaCa | NaCa | [symbol] | [symbol] | A/A | A/A | A/A | A/A | [symbol] |
| C___ [symbol] _ [symbol] _ | [symbol] | [symbol] | [symbol] | C/G | [symbol] | [symbol] | [symbol] | [symbol] | C/G | C/G | C/G | C/G | [symbol] |
| C___ [symbol] _ [symbol] | [symbol] | [symbol] | [symbol] | G/G | [symbol] | [symbol] | [symbol] | [symbol] | G/G | G/G | G/G | G/G | [symbol] |
| C___ [symbol] _ [symbol] | [symbol] | [symbol] | [symbol] | G/G | [symbol] | [symbol] | [symbol] | [symbol] | G/G | G/G | G/G | G/G | [symbol] |
| C___ [symbol] _ [symbol] _ | [symbol] | [symbol] | [symbol] | A/A | [symbol] | [symbol] | NaCa | NaCa | A/A | A/A | A/A | A/A | [symbol] |
| C___ [symbol] _ [symbol] | [symbol] | [symbol] | [symbol] | A/G | [symbol] | [symbol] | [symbol] | [symbol] | G/G | A/G | A/A | A/G | [symbol] |
| C___ [symbol] _ [symbol] | [symbol] | [symbol] | [symbol] | A/A | [symbol] | [symbol] | [symbol] | [symbol] | A/A | A/A | A/A | A/A | [symbol] |
| C___ [symbol] _ [symbol] | [symbol] | [symbol] | [symbol] | NaCa | [symbol] | [symbol] | [symbol] | [symbol] | NaCa | A/A | A/A | A/A | [symbol] |
| C___ [symbol] _ [symbol] | [symbol] | [symbol] | [symbol] | C/T | [symbol] | [symbol] | [symbol] | [symbol] | C/T | C/T | C/T | C/T | [symbol] |
| C___ [symbol] _ [symbol] | [symbol] | [symbol] | [symbol] | A/G | [symbol] | [symbol] | [symbol] | [symbol] | A/G | A/G | A/G | A/G | [symbol] |
| C___ [symbol] _ [symbol] | [symbol] | [symbol] | [symbol] | A/G | [symbol] | [symbol] | [symbol] | [symbol] | G/G | G/G | A/A | A/A | [symbol] |
| C___ [symbol] _ [symbol] | [symbol] | [symbol] | [symbol] | C/C | [symbol] | [symbol] | [symbol] | [symbol] | C/C | C/C | C/C | C/C | [symbol] |
| C___ [symbol] _ [symbol] | [symbol] | [symbol] | [symbol] | A/T | A/A | A/A | [symbol] | [symbol] | A/A | A/A | A/A | A/T | [symbol] |
| C___ [symbol] _ [symbol] | [symbol] | [symbol] | [symbol] | G/G | A/A | A/A | [symbol] | [symbol] | A/G | A/G | A/G | A/G | [symbol] |
| Number of Miss-calls | | | | 1 | 2 | 2 | 0 | 0 | 2 | 1 | 2 | 0 | |
| C____ [symbol] _ [symbol] | [symbol] | [symbol] | [symbol] | A/G | [symbol] | [symbol] | A/A | A/A | A/A | A/A | A/G | A/G | [symbol] |
| C___ [symbol] _ [symbol] _ | [symbol] | [symbol] | [symbol] | A/G | G/G | [symbol] | G/G | G/G | A/A | A/A | A/A | A/A | [symbol] |
| C___ [symbol] _ [symbol] | [symbol] | [symbol] | [symbol] | A/A | [symbol] | [symbol] | [symbol] | [symbol] | A/C | A/A | A/A | A/A | [symbol] |
| C___ [symbol] _ [symbol] _ | [symbol] | [symbol] | [symbol] | C/G | [symbol] | [symbol] | [symbol] | [symbol] | C/G | C/G | C/G | C/G | [symbol] |
| C___ [symbol] _ [symbol] | [symbol] | [symbol] | [symbol] | A/G | [symbol] | [symbol] | [symbol] | [symbol] | G/G | A/G | A/G | A/G | [symbol] |
| C___ [symbol] _ [symbol] | [symbol] | [symbol] | [symbol] | A/A | [symbol] | [symbol] | [symbol] | [symbol] | A/A | A/A | A/A | A/A | [symbol] |
| C___ [symbol] _ [symbol] _ | [symbol] | [symbol] | [symbol] | A/G | [symbol] | [symbol] | [symbol] | [symbol] | G/G | G/G | G/G | A/G | [symbol] |
| C___ [symbol] _ [symbol] | [symbol] | [symbol] | [symbol] | G/G | [symbol] | [symbol] | [symbol] | [symbol] | G/G | G/G | A/G | G/G | [symbol] |
| C___ [symbol] _ [symbol] | [symbol] | [symbol] | [symbol] | A/A | [symbol] | [symbol] | [symbol] | [symbol] | A/A | A/A | A/A | A/A | [symbol] |
| C___ [symbol] _ [symbol] | [symbol] | [symbol] | [symbol] | G/G | [symbol] | [symbol] | [symbol] | [symbol] | NaCa | NaCa | G/G | G/G | [symbol] |
| C___ [symbol] _ [symbol] | [symbol] | [symbol] | [symbol] | C/T | [symbol] | [symbol] | [symbol] | [symbol] | T/T | T/T | C/C | C/C | [symbol] |
| C___ [symbol] _ [symbol] | [symbol] | [symbol] | [symbol] | A/G | [symbol] | [symbol] | [symbol] | [symbol] | G/G | G/G | A/G | A/G | [symbol] |
| C___ [symbol] _ [symbol] | [symbol] | [symbol] | [symbol] | A/A | [symbol] | [symbol] | [symbol] | [symbol] | A/A | A/A | A/A | A/A | [symbol] |
| C___ [symbol] _ [symbol] | [symbol] | [symbol] | [symbol] | C/T | [symbol] | [symbol] | T/T | T/T | C/T | C/T | T/T | T/T | [symbol] |
| C___ [symbol] _ [symbol] | [symbol] | [symbol] | [symbol] | A/T | [symbol] | [symbol] | [symbol] | [symbol] | A/T | A/T | T/T | T/T | [symbol] |
| C___ [symbol] _ [symbol] | [symbol] | [symbol] | [symbol] | G/G | [symbol] | [symbol] | [symbol] | [symbol] | G/G | G/G | G/G | G/G | [symbol] |
| Number of Miss-calls | | | | 0 | 1 | 0 | 3 | 3 | 1 | 1 | 2 | 2 | [symbol] |

[symbol]

indicates data missing or illegible when filed

In sum, these data show that the pre-amplification and Open Array genotyping assay provide an average test accuracy of 98.9% and an average reproducibility of 98.3%.

These data indicate that experimental error can cause genotype result mismatch for 1 to 3 markers. Therefore, these data suggest that a definitive negative match should not be made when only 1-3 markers are mismatched between the buccal cell sample and the urine DNA sample. These samples may be further tested using the 384-well format assay described in the Examples above.

### Example 5. Spectrophotometric Identification of Synthetic Urine

An additional set of experiments were performed to determine whether ultraviolet light absorbance of a urine sample could be used to accurately determine if a urine sample contained synthetic urine.

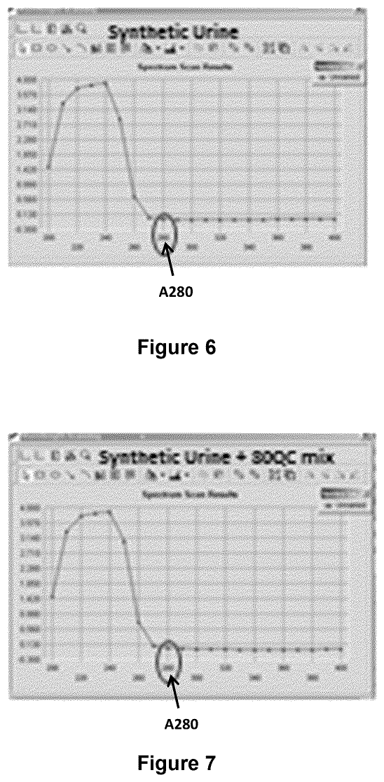

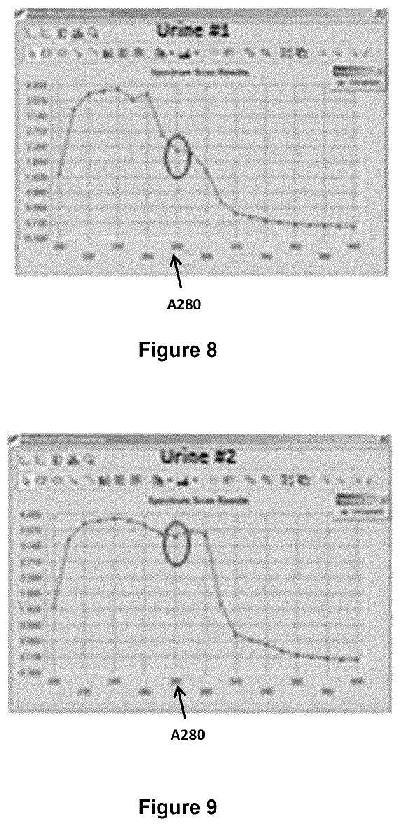

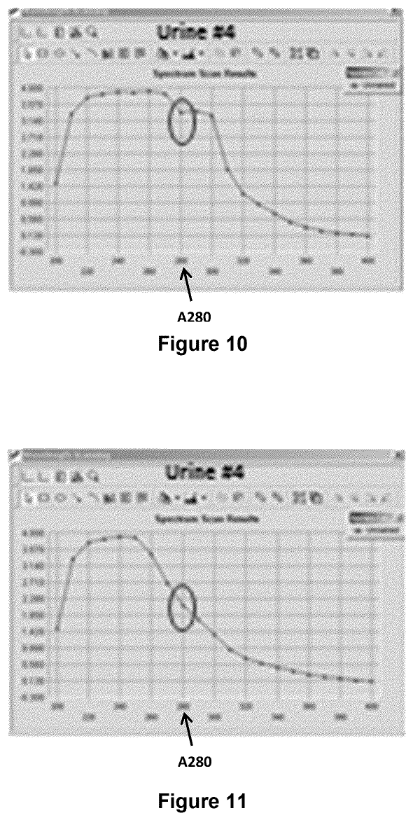

A first experiment was performed to obtain the light absorption spectrum of synthetic urine, synthetic urine plus drugs and their metabolites (used as a quality control; 5 μL of a drug metabolite positive control in 100 μL of synthetic urine), and urine samples from 4 individuals. The absorbance spectra are shown in FIGS. 6-11. The data in FIG. 6 show that the light absorbance of the synthetic urine peaked at 240 nm and then dropped to near zero absorbance at 280 nm. The addition of the quality control of the drug and drug metabolite mix did not change the light absorbance profile of the synthetic urine. The light absorbance profile of urine samples (originating from a human patient) varies between different individuals. However the OD280 value for the urine sample (originating from a human patient) were much higher than that of the synthetic urine, with the lowest value well above 1.8 absorbance units.

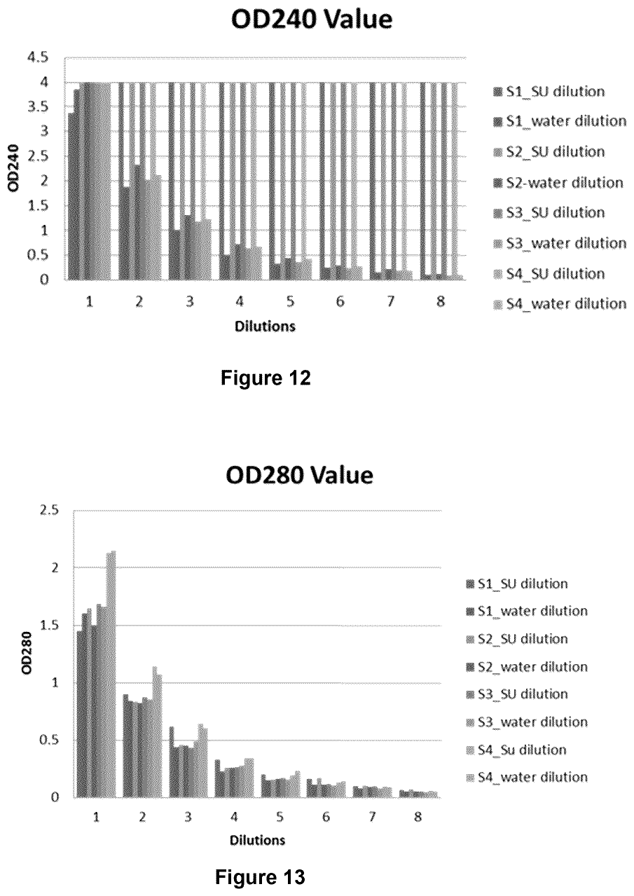

A second set of experiments were performed to determine whether the absorbance at 280 nm can be used to identify urine samples diluted with water or synthetic urine. In these experiments, a two-fold dilution series was made for 4 urine samples with synthetic urine and water, respectively. Negative controls of urine free samples or blank samples were included. The samples were then measured for OD240 and OD280. The results of the OD240 and OD280 values are shown in Table 25 and shown in FIGS. 12 and 13.

TABLE 25
The OD240 and OD280 Values for Serial 2-Fold Dilutions

| | VGTX0038 | | VGTX0007 | | VGTX0004_sp | | VGTX0004 | | |
|---|---|---|---|---|---|---|---|---|---|
| OD240 | Syn Urine | Water | Syn Urine | Water | Syn Urine | Water | Syn Urine | Water | |
| 1 | 3.365 | 3.856 | 4 | 4 | 4 | 4 | 4 | 4 | |
| ½ | 4 | 1.884 | 4 | 2.324 | 4 | 2.035 | 4 | 2.119 | |
| ¼ | 4 | 0.998 | 4 | 1.3 | 4 | 1.181 | 4 | 1.22 | |
| ⅛ | 4 | 0.504 | 4 | 0.715 | 4 | 0.639 | 4 | 0.666 | |
| 1/16 | 4 | 0.33 | 4 | 0.443 | 4 | 0.364 | 4 | 0.426 | |
| 1/32 | 4 | 0.241 | 4 | 0.29 | 4 | 0.231 | 4 | 0.274 | |
| 1/64 | 4 | 0.155 | 4 | 0.205 | 4 | 0.173 | 4 | 0.188 | |
| Blank | 4 | 0.101 | 4 | 0.113 | 4 | 0.09 | 4 | 0.104 | |
| OD280 | | | | | | | | | Mean |
| 1 | 1.447 | 1.601 | 1.648 | 1.494 | 1.686 | 1.663 | 2.131 | 2.151 | 1.728 |
| ½ | 0.898 | 0.841 | 0.834 | 0.821 | 0.874 | 0.857 | 1.145 | 1.073 | 0.918 |
| ¼ | 0.616 | 0.436 | 0.456 | 0.451 | 0.435 | 0.49 | 0.642 | 0.604 | 0.516 |
| ⅛ | 0.332 | 0.23 | 0.257 | 0.258 | 0.268 | 0.28 | 0.342 | 0.344 | 0.289 |
| 1/16 | 0.205 | 0.152 | 0.16 | 0.164 | 0.171 | 0.159 | 0.196 | 0.232 | 0.180 |
| 1/32 | 0.162 | 0.113 | 0.169 | 0.111 | 0.118 | 0.104 | 0.133 | 0.145 | 0.132 |
| 1/64 | 0.099 | 0.079 | 0.105 | 0.095 | 0.098 | 0.08 | 0.098 | 0.095 | 0.094 |
| Blank | 0.067 | 0.054 | 0.074 | 0.054 | 0.058 | 0.046 | 0.06 | 0.058 | 0.059 |

The data in Table 25 and FIGS. 12 and 13 show that the value of OD240 stays constant throughout the dilution series with synthetic urine for all 4 samples. For the dilution series with water, the OD240 value shows a linear reduction. A linear reduction of OD280 values are observed for both the urine dilution series with synthetic urine and with water. These data indicate that it is possible to use OD240 and OD280 measurement to identify samples altered by dilution.

A third set of experiments were performed to test 377 urine samples for OD240 and OD280 distribution. Out of the 377 samples tested, 16 samples have OD280 values below 1 (4% of the total tested samples). These samples also have a low OD240 value indicating sample dilution with water (Table 26). The remaining 361 samples (96%) have an OD280 value of greater than 1.00.

TABLE 26
OD240 and OD280 Levels of Potentially Diluted Samples

| # | Sample ID | OD240 | OD280 |
|---|---|---|---|
| 1 | 1405160017 | 2.218 | 0.1 |
| 2 | 1405190017 | 2.354 | 0.125 |
| 3 | 1405140004 | 1.536 | 0.392 |
| 4 | 1405150038 | 1.89 | 0.618 |
| 5 | 1405010008 | 1.403 | 0.627 |
| 6 | 1405150005 | 1.579 | 0.631 |
| 7 | 1405130005 | 1.257 | 0.663 |
| 8 | 1405140003 | 1.66 | 0.75 |
| 9 | 1405160015 | 1.596 | 0.765 |
| 10 | 1405050016 | 3.518 | 0.84 |
| 11 | 1405050028 | 2.191 | 0.864 |
| 12 | 1405140010 | 1.84 | 0.882 |
| 13 | 1405210011 | 3.221 | 0.92 |
| 14 | 1405050026 | 2.994 | 0.94 |
| 15 | 140150029 | 2.956 | 0.956 |
| 16 | 1405010019 | 2.288 | 1.001 |

Based on these data, we conclude that the absorbance at 240 nm and 280 nm can be used to effectively and accurately identify urine samples containing synthetic urine. The absorbance at 240 nm and 280 nm can also be used to detect dilution of a urine produced by a subject's body with water or synthetic urine.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining if a urine sample comprises synthetic urine comprising:
(a) providing a urine sample from a subject;
(b) enriching the urine sample for mammalian cells, if present;
(c) isolating any genomic DNA from the enriched sample of step (b) to form an isolated genomic DNA test sample;
(d) adding to the isolated genomic DNA test sample of step (c) a control DNA to form a control sample or adding the control DNA to the enriched sample of step (b) and then isolating DNA to form a control sample;
(e) performing an assay to determine the presence of genomic DNA in the isolated genomic DNA sample of step (c) or the control sample of step (d);
(f) performing an assay to determine the presence of the control DNA in the control sample of step (d); and
(g) identifying a urine sample having no detectable level of genomic DNA and having detectable control DNA as containing synthetic urine, or identifying a urine sample having a detectable level of genomic DNA and having detectable control DNA as not comprising a synthetic urine.

2. The method of claim 1, wherein:
the determination of the presence of genomic DNA comprises performing an assay to determine the presence of at least three single nucleotide polymorphisms in the isolated genomic DNA sample of step (c) or the control sample of step (d), and
a urine sample having no detectable level of the at least three SNPs and having detectable control DNA is identified in step (g) as containing synthetic urine, or a urine sample having a detectable level of the at least three SNPs and having detectable control DNA is identified in step (g) as not comprising synthetic urine.

3. The method of claim 2, wherein the urine sample is identified in step (g) as not comprising synthetic urine.

4. (canceled)

5. The method of claim 3, further comprising:
(h) performing an assay to determine the genotype of at least 6 single nucleotide polymorphisms (SNPs) in the isolated genomic DNA test sample of step (c) or the control sample of step (d);
(i) comparing the genotype of the at least 6 SNPs in the isolated genomic DNA test sample of step (c) or the control sample of step (d) with the genotype of the at least 6 SNPs in a control cell sample from the subject; and
(j) identifying a urine sample having a detectable level of the control DNA and having the same genotype of the at least 6 SNPs in the isolated genomic DNA test sample of step (c) or the control sample of step (d) as the genotype of the at least 6 SNPs in the control cell sample as originating from the subject; or
identifying a urine sample having a detectable level of the control DNA and not having the same genotype of the at least 6 SNPs in the isolated genomic DNA test sample of (c) or the control sample of step (d) as the genotype of the at least 6 SNPs in the control cell sample as not originating from the subject.

6.-17. (canceled)

18. The method of claim 2, wherein the assay in step (e) comprises a pre-amplification step.

19. The method of claim 18, wherein the pre-amplification step includes: hybridization of three or more pairs of a pre-amplification forward and reverse primer, wherein each pair of pre-amplification forward and reverse primers is designed to amplify 250 to 300 nucleotides of genomic DNA that contains one of the at least 3 SNPs, wherein the pre-amplification forward and reverse primers in each of the three or more pairs of pre-amplification primers contain (i) a sequence of about 17 to about 25 contiguous nucleotides that is complementary to a sequence in the genomic DNA and (ii) a tag sequence of about 17 to about 25 contiguous nucleotides that is not complementary to a sequence in the genomic DNA; and amplification of the genomic DNA using the three or more pairs of pre-amplification forward and reverse primers to generate 250 to 300 nucleotide amplification product(s).

20. The method of claim 19, wherein the pre-amplification step further comprises amplification of the 250 to 300 nucleotide amplification product(s) using a primer that comprises a sequence of about 17 to about 25 contiguous nucleotides of the tag sequence.

21.-28. (canceled)

29. The method of claim 1, further comprising:
(h) performing an assay to identify the presence of one or more of statherin, alpha-amylase, and lysozyme in the urine sample; and
(i) identifying a urine sample having a detectable level of genomic DNA, a detectable control DNA, and a detectable level of one or more of statherin, alpha-amylase, and lysozyme as being adulterated.

30.-35. (canceled)

36. The method of claim 1, further comprising:
(h) selecting a subject having a urine sample identified in step (g) as containing synthetic urine; and
(i) obtaining an additional urine sample from the selected subject.

37. (canceled)

38. The method of claim 36, further comprising:
(j) performing an assay to determine the level of one or more drugs and/or the level of one or more drug metabolites in the additional urine sample.

39. The method of claim 38, further comprising:
(k) identifying a subject having an elevated level of one or more drugs and/or an elevated level of one or more drug metabolites in the additional urine sample as compared to a reference level of the one or more drugs and/or a reference level of the one or more drug metabolites, wherein the drugs are an illegal or controlled substance and/or the drug metabolites are metabolites of an illegal or controlled substance; and
(l) admitting the subject into a drug dependency program, ceasing administration of the controlled substance to the subject, or reducing the dose and/or frequency of administration of the controlled substance to the subject.

40. (canceled)

41. The method of claim 1, further comprising:
(h) selecting a subject having a urine sample identified in step (g) as containing synthetic urine;
(i) obtaining a sample comprising blood, serum, hair, or plasma from the subject; and
(j) performing an assay to determine the level of one or more drugs and/or one or more drug metabolites in the sample from step (i).

42. The method of claim 41, further comprising:
(k) identifying a subject having an elevated level of one or more drugs and/or an elevated level of one or more drug metabolites in the sample from step (i) as compared to a reference level of the one or more drugs and/or a reference level of the one or more drug metabolites, wherein the drugs are an illegal or controlled substance and/or the drug metabolites are metabolites of an illegal or controlled substance; and
(l) admitting the subject into a drug dependency program, ceasing administration of the controlled substance to the subject, or reducing the dose or frequency of administration of the controlled substance to the subject.

43.-46. (canceled)

47. A method of determining if a urine sample comprises synthetic urine and/or is diluted comprising:
(a) providing a urine sample from a subject;
(b) detecting the absorbance at 280 nm of the urine sample; and
(c) identifying a urine sample having an absorbance at 280 nm that is less than a reference 280 nm absorbance value as comprising synthetic urine and/or being diluted, or identifying a urine sample having an absorbance at 280 nm that is equal to or greater than the reference 280 nm absorbance value as not comprising synthetic urine and not being diluted.

48. (canceled)

49. The method of claim 47, further comprising:
(d) determining the absorbance at 240 nm of the urine sample; and
(e) further identifying a urine sample having an absorbance at 280 nm that is less than a reference 280 nm absorbance value and an absorbance at 240 nm that is less than a reference 240 nm absorbance value as being diluted.

50.-104. (canceled)

105. The method of claim 47, further comprising:
(d) selecting a subject having a urine sample identified in step (c) as comprising synthetic urine and/or being diluted;
(e) obtaining an additional sample comprising blood, serum, hair, or plasma from the subject; and
(f) performing an assay to determine the level of one or more drugs and/or the level of one or more drug metabolites in the additional sample from step (e).

106.-112. (canceled)

113. A method of matching a urine sample to a subject comprising:
(a) providing a urine sample from a subject;
(b) enriching the urine sample for mammalian cells, if present;
(c) isolating any genomic DNA from the enriched sample of step (b) to form an isolated genomic DNA test sample;
(d) adding to the isolated genomic DNA test sample of step (c) a control DNA to form a control sample or adding the control DNA to the enriched sample of step (b) and then isolating the DNA to form a control sample;
(e) performing an assay to determine the genotype of at least 6 single nucleotide polymorphisms (SNPs) in the isolated genomic DNA test sample of step (c) or the control sample of step (d);
(f) comparing the genotype of the at least 6 SNPs in the isolated genomic DNA test sample of step (c) or the control sample of step (d) with the genotype of the at least 6 SNPs in a control cell sample from the subject;
(g) performing an assay to determine the presence of the control DNA in the control sample of step (d); and
(h) identifying a urine sample having a detectable level of the control DNA and having the same genotype of the at least 6 SNPs in the isolated genomic DNA test sample of step (c) or the control sample of step (d) as the genotype of the at least 6 SNPs in the control cell sample as originating from the subject; or
identifying a urine sample having a detectable level of the control DNA and not having the same genotype of the at least 6 SNPs in the isolated genomic DNA test sample of step (c) or the control sample of step (d) as the genotype of the at least 6 SNPs in the control cell sample as not originating from the subject.

114.-147. (canceled)

148. The method of claim 113, further comprising:
(i) performing an assay to identify the presence of one or more of statherin, alpha-amylase, and lysozyme in the urine sample; and
(j) identifying a urine sample having a genotype of the at least 6 SNPs in the isolated genomic DNA test sample of step (c) or the control sample of step (d) that is the same as the genotype of the 6 SNPs in the control cell sample, a detectable level of control DNA, and a detectable level of one or more of statherin, alpha-amylase, and lysozyme as being adulterated.

149.-193. (canceled)


SEQUENCE LISTING (41 sequences)

SEQ ID NO: 1 - DNA, 20 bp, synthetic construct
  misc_feature 1..20: note = synthetic oligonucleotide primer
  source 1..20: mol_type = other DNA
caagatgctacgcttcagtc

SEQ ID NO: 2 - DNA, 43 bp, synthetic construct
  misc_feature 1..43: note = synthetic oligonucleotide primer
  source 1..43: mol_type = other DNA
caagatgctacgcttcagtcaggagactatgaggtgtgtctct

SEQ ID NO: 3 - DNA, 39 bp, synthetic construct
  misc_feature 1..39: note = synthetic oligonucleotide primer
  source 1..39: mol_type = other DNA
caagatgctacgcttcagtccctgtgcacctcgattgaa

SEQ ID NO: 4 - DNA, 39 bp, synthetic construct
  misc_feature 1..39: note = synthetic oligonucleotide primer
  source 1..39: mol_type = other DNA
caagatgctacgcttcagtcccaaggggaatcacacctc

SEQ ID NO: 5 - DNA, 40 bp, synthetic construct
  misc_feature 1..40: note = synthetic oligonucleotide primer
  source 1..40: mol_type = other DNA
caagatgctacgcttcagtctctgtggaagcatgccactc

SEQ ID NO: 6 - DNA, 40 bp, synthetic construct
  misc_feature 1..40: note = synthetic oligonucleotide primer
  source 1..40: mol_type = other DNA
caagatgctacgcttcagtctgctgagccactctttcagg

SEQ ID NO: 7 - DNA, 40 bp, synthetic construct
  misc_feature 1..40: note = synthetic oligonucleotide primer
  source 1..40: mol_type = other DNA
caagatgctacgcttcagtcttccgggatgtcccgtctta

SEQ ID NO: 8 - DNA, 43 bp, synthetic construct
  misc_feature 1..43: note = synthetic oligonucleotide primer
  source 1..43: mol_type = other DNA
caagatgctacgcttcagtcacaggcaaagaggaacatacagt

SEQ ID NO: 9 - DNA, 44 bp, synthetic construct
  misc_feature 1..44: note = synthetic oligonucleotide primer
  source 1..44: mol_type = other DNA
caagatgctacgcttcagtctgctggcagtgttatttctttctc

SEQ ID NO: 10 - DNA, 37 bp, synthetic construct
  misc_feature 1..37: note = synthetic oligonucleotide primer
  source 1..37: mol_type = other DNA
caagatgctacgcttcagtcctcgttgttcctctggg

SEQ ID NO: 11 - DNA, 39 bp, synthetic construct
  misc_feature 1..39: note = synthetic oligonucleotide primer
  source 1..39: mol_type = other DNA
caagatgctacgcttcagtcctgttcaagggaagcctgt

SEQ ID NO: 12 - DNA, 40 bp, synthetic construct
  misc_feature 1..40: note = synthetic oligonucleotide primer
  source 1..40: mol_type = other DNA
caagatgctacgcttcagtcggatcaggaaacagggagcc

SEQ ID NO: 13 - DNA, 40 bp, synthetic construct
  misc_feature 1..40: note = synthetic oligonucleotide primer
  source 1..40: mol_type = other DNA
caagatgctacgcttcagtcgaagactctgtcccagccac

SEQ ID NO: 14 - DNA, 40 bp, synthetic construct
  misc_feature 1..40: note = synthetic oligonucleotide primer
  source 1..40: mol_type = other DNA
caagatgctacgcttcagtcgcttcttctgccacatccct

SEQ ID NO: 15 - DNA, 40 bp, synthetic construct
  misc_feature 1..40: note = synthetic oligonucleotide primer
  source 1..40: mol_type = other DNA
caagatgctacgcttcagtcgcattttcatggagggccac

SEQ ID NO: 16 - DNA, 40 bp, synthetic construct
  misc_feature 1..40: note = synthetic oligonucleotide primer
  source 1..40: mol_type = other DNA
caagatgctacgcttcagtcttgccatgtttgtcacaggt

SEQ ID NO: 17 - DNA, 45 bp, synthetic construct
  misc_feature 1..45: note = synthetic oligonucleotide primer
  source 1..45: mol_type = other DNA
caagatgctacgcttcagtcaccttggtttcttgattatgttgat

SEQ ID NO: 18 - DNA, 39 bp, synthetic construct
  misc_feature 1..39: note = synthetic oligonucleotide primer
  source 1..39: mol_type = other DNA
caagatgctacgcttcagtctgaatcctcccccaagctg

SEQ ID NO: 19 - DNA, 40 bp, synthetic construct
  misc_feature 1..40: note = synthetic oligonucleotide primer
  source 1..40: mol_type = other DNA
caagatgctacgcttcagtcttctcctcttcctgggctga

SEQ ID NO: 20 - DNA, 40 bp, synthetic construct
  misc_feature 1..40: note = synthetic oligonucleotide primer
  source 1..40: mol_type = other DNA
caagatgctacgcttcagtcgcacattaaatgggttccag

SEQ ID NO: 21 - DNA, 44 bp, synthetic construct
  misc_feature 1..44: note = synthetic oligonucleotide primer
  source 1..44: mol_type = other DNA
caagatgctacgcttcagtcgaaatacgaaggacacaaaacctc

SEQ ID NO: 22 - DNA, 40 bp, synthetic construct
  misc_feature 1..40: note = synthetic oligonucleotide primer
  source 1..40: mol_type = other DNA
caagatgctacgcttcagtctgttgctggcaagacacttc

SEQ ID NO: 23 - DNA, 41 bp, synthetic construct
  misc_feature 1..41: note = synthetic oligonucleotide primer
  source 1..41: mol_type = other DNA
caagatgctacgcttcagtctgcctttgctacaactctcct

SEQ ID NO: 24 - DNA, 39 bp, synthetic construct
  misc_feature 1..39: note = synthetic oligonucleotide primer
  source 1..39: mol_type = other DNA
caagatgctacgcttcagtctagccccacgtcacttcag

SEQ ID NO: 25 - DNA, 40 bp, synthetic construct
  misc_feature 1..40: note = synthetic oligonucleotide primer
  source 1..40: mol_type = other DNA
caagatgctacgcttcagtcttcttggaaggtggtcctgg

SEQ ID NO: 26 - DNA, 43 bp, synthetic construct
  misc_feature 1..43: note = synthetic oligonucleotide primer
  source 1..43: mol_type = other DNA
caagatgctacgcttcagtcaggacctgtaagagtctgtgatt

SEQ ID NO: 27 - DNA, 43 bp, synthetic construct
  misc_feature 1..43: note = synthetic oligonucleotide primer
  source 1..43: mol_type = other DNA
caagatgctacgcttcagtctgtatcccaggttcaatgactgt

SEQ ID NO: 28 - DNA, 43 bp, synthetic construct
  misc_feature 1..43: note = synthetic oligonucleotide primer
  source 1..43: mol_type = other DNA
caagatgctacgcttcagtcacacccttacctgtattttctga

SEQ ID NO: 29 - DNA, 44 bp, synthetic construct
  misc_feature 1..44: note = synthetic oligonucleotide primer
  source 1..44: mol_type = other DNA
caagatgctacgcttcagtcgtgcacattctaagaactggtgat

SEQ ID NO: 30 - DNA, 41 bp, synthetic construct
  misc_feature 1..41: note = synthetic oligonucleotide primer
  source 1..41: mol_type = other DNA
caagatgctacgcttcagtctgcttacagtgattcttgcct

SEQ ID NO: 31 - DNA, 43 bp, synthetic construct
  misc_feature 1..43: note = synthetic oligonucleotide primer
  source 1..43: mol_type = other DNA
caagatgctacgcttcagtcagtcttttgcaccaagtcttttt

SEQ ID NO: 32 - DNA, 41 bp, synthetic construct
  misc_feature 1..41: note = synthetic oligonucleotide primer
  source 1..41: mol_type = other DNA
caagatgctacgcttcagtcggaagatgcttgaactcccca

SEQ ID NO: 33 - DNA, 39 bp, synthetic construct
  misc_feature 1..39: note = synthetic oligonucleotide primer
  source 1..39: mol_type = other DNA
caagatgctacgcttcagtcacatcaaagctgggaaccg

SEQ ID NO: 34-35 - skipped (2 entries)

SEQ ID NO: 36 - DNA, 22 bp, synthetic construct
  misc_feature 1..22: note = forward AtpC primer
  source 1..22: mol_type = other DNA
tccctccttatccatccttaca

SEQ ID NO: 37 - DNA, 21 bp, synthetic construct
  misc_feature 1..21: note = reverse AtpC primer
  source 1..21: mol_type = other DNA
cagagagaagggtgtgatgtg

SEQ ID NO: 38 - protein, 62 aa, Homo sapiens
MKFLVFAFILALMVSMIGADSSEEKFLRRIGRFGYGYGPYQPVPEQPLYPQPYQPQYQQYTF

SEQ ID NO: 39 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 40 - protein, 148 aa, Homo sapiens
MKALIVLGLVLLSVTVQGKVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 41 - DNA, 24 bp, synthetic construct
  misc_feature 1..24: note = spinach AtpC probe
  source 1..24: mol_type = other DNA
tccacaattccaacaccctcctcc